(19) United States
(12) Patent Application Publication
(10) Pub. No.: US 2023/0371507 A1
(43) Pub. Date: Nov. 23, 2023

(54) PESTICIDAL COMPOUNDS AND COMPOSITIONS, METHODS OF USE AND PROCESSES OF PREPARATION THEREOF

(71) Applicants: Noam Sheffer, Herzliya (IL); Limor Poraty-Gavra, Rehovot (IL); Itsik Bar Nahum, Kfar-Gibton (IL); Sami Shabtai, Omer (IL); Dusan Goranovic, Ljubljana (SI); Gregor Kosec, Ljubljana (SI); Leon Bedrac, Gorisnica (SI); Alen Cusak, Ljubljana (SI); Adama Makhteshim Ltd., Beer Sheva (IL)

(72) Inventors: Noam Sheffer, Herzliya (IL); Limor Poraty-Gavra, Rehovot (IL); Itsik Bar Nahum, Kfar-Gibton (IL); Sami Shabtai, Omer (IL); Dusan Goranovic, Ljubljana (SI); Gregor Kosec, Ljubljana (SI); Leon Bedrac, Gorisnica (SI); Alen Cusak, Ljubljana (SI)

(73) Assignee: Adama Makhteshim Ltd., Beer Sheva (IL)

(21) Appl. No.: 18/042,728

(22) PCT Filed: Aug. 24, 2021
(86) PCT No.: PCT/IB2021/057761

§ 371 Date: Feb. 23, 2023

Related U.S. Application Data
(60) Provisional application No. 63/069,526, filed on Aug. 24, 2020.

(51) Int. Cl. A01N 37/06; C07C 51/09; A01N 63/32; A01P 3/00; C12P 7/44

(52) U.S. Cl. CPC A01N 37/06; C07C 51/09; A01N 63/32; A01P 3/00; C12P 7/44

(57) ABSTRACT

The present invention provides a method of treating a locus against pest infestation using a compound of Formula I as described herein or an agriculturally acceptable salt thereof. The present invention also provides combinations and compositions comprising the compound of Formula I or an agriculturally acceptable salt thereof and uses thereof. The present invention also provides methods of extracting the compound of Formula I from a fungus.

### FIELD OF THE INVENTION

The present subject matter relates to the pesticidal activity of compounds of Formula I, especially 2-octyl-2-pentenedioic acid.

### BACKGROUND AND SUMMARY

Fungicides and bactericides are compounds, of natural or synthetic origin, which act to protect plants against damage caused by fungi and bacteria, respectively. Current methods of agriculture rely heavily on the use of fungicides and/or bactericides. In fact, some crops cannot be grown usefully without the use of fungicides and/or bactericides. Using fungicides and/or bactericides allows a grower to increase the yield and the quality of the crop, and consequently, increase the value of the crop.

Endo, A. et al. (1985). Acetyl CoA carboxylase inhibitors from the fungus Gongronella Butreli. The Journal of J Antibiotics, 38(5): 599-604 discuss specific compounds having a structure of pentanedioic acid or pentenedioic acid which were isolated from Gongronella butleri and their inhibitory effects on rat liver acetyl CoA carboxylase.

Akone, S. H. et al. (2014). 2-Pentenedioic acid derivatives from a soil-derived fungus Gongronella butleri. Phytochemistry Letters, 10: 184-188 discuss new 2-pentenedioic acid derivatives which were isolated from Gongronella butleri. The compounds were tested for their antibacterial activity against Staphyloccocus aureus, Steptococcus pneumonia and Escherichia coli, and no activity was observed.

Many fungicides and bactericides are not useful in all situations and repeated usage of a single fungicide or bactericide frequently leads to the development of resistance to the related fungicides or bactericides. Accordingly, research is being conducted to discover fungicides and bactericides and combinations thereof that have improved safety, lower cost, better performance, lower dosage requirements and easier use.

### SUMMARY OF THE INVENTION

The present invention provides a method of treating a locus against pest infestation comprising applying an effective amount of at least one compound of Formula I:

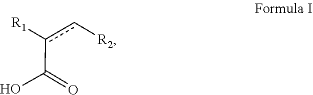

wherein
R₁ is a C₁-C₁₂ alkyl optionally substituted with one or more substituents independently selected from the group consisting of halogen, C(O)R, CN, NH₂, —NH(C₁-C₄ alkyl), —(C₁-C₄ alkyl)—NH₂, —N(C₂-C₄ alkyl)(C₂-C₄ alkyl), OR, —S(O)₂(C₁-C₄ alkyl), —S(O)(C₁-C₄ alkyl), C(O)OH and C(O)OR,
R₂ is —(C₁-C₁₂)—COOH, —(C₁-C₁₂)—COO, —(C₁-C₁₂)—COOR, —(C₁-C₁₂)—CONH₂, —(C₁-C₁₂)—CONHR, —(C₁-C₁₂)—CONR₂, —(C₁-C₁₂)—COSR, —(C₁-C₁₂)—COX, —(C₁-C₁₂)—S(O)₂NH₂, —(C₁-C₁₂)—S(O)₂NHR, —(C₁-C₁₂)—S(O)₂NR₂, —(C₁-C₁₂)—CN, —(C₁-C₁₂)—CR═NR, —(C₁-C₁₂)—CONHNH₂, wherein each of the —(C₁-C₁₂)—COOH, —(C₁-C₁₂)—COO⁻, —(C₁-C₁₂)—COOR, —(C₁-C₁₂)—CONH₂, —(C₁-C₂)—CONHR, —(C₁-C₁₂)—CONR₂, —(C₁-C₁₂)—COSR, —(C₁-C₁₂)—COX, —(C₁-C₁₂)—S(O)₂NH₂, —(C₁-C₂)—S(O)₂NHR, —(C₁-C₂)—S(O)₂NR₂, —(C₁-C₁₂)—CN, —(C₁-C₁₂)—CR═NR, and —(C₁-C₁₂)—CONHNH₂ may be optionally substituted with halogen, amine, carboxylic acid, OR, CN, a C₁-C₁₂ thioester, and/or sulphonyl,
R is independently H or an optionally substituted C₁-C₁₂ alkyl group,
X is halogen, and
[symbol] is an optional double bond;
   or an agriculturally acceptable salt thereof,
   to the locus so as to thereby treat the locus against pest infestation, wherein the compound of Formula I is (i) produced synthetically, (ii) applied free of fungal material from Pseudozyma aphid, and/or (iii) applied at an amount such that the locus being treated is exposed to an amount of the compound of Formula I that is higher than the amount the locus would be exposed to from secretions of Pseudozyma aphidis that exist naturally at the locus or is introduced artificially to the locus.

The present invention provides a method of treating a locus against pest infestation comprising applying an effective amount of at least one compound of Formula I:

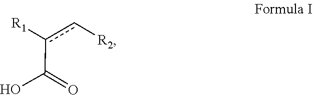

wherein
R₁ is a C₁-C₁₂ alkyl optionally substituted with one or more substituents independently selected from the group consisting of halogen, CN, NH₂, C₁-C₄ alkylamino, C₂-C₄ dialkylamino, OR, C₁-C₄ alkylsulfonyl, C₁-C₄ alkylsulfinyl, C(O)OH, C(O)OR,
R₂ is a C₁-C₁₂ (a) carboxylic acid, (b) amide, (c) thioester, (d) acyl halide, (e) sulfonyl amide, wherein each of the C₁-C₁₂ carboxylic acid, amid, thioester, acyl halide and sulfonyl amide may be optionally substituted with halogen, amine, carboxylic acid, OR, CN, a C₁-C₁₂ thioester, and/or sulphonyl,
R is H or an optionally substituted C₁-C₁₂ alkyl group, and
[symbol] is an optional double bond;

or an agriculturally acceptable salt thereof,

to the locus so as to thereby treat the locus against pest infestation, wherein the compound of Formula I is (i) produced synthetically, (ii) applied free of fungal material from Pseudozyma aphidis, and/or (iii) applied at an amount such that the locus being treated is exposed to an amount of the compound of Formula I that is higher than the amount the locus would be exposed to from secretions of Pseudozyma aphidis that exist naturally at the locus or is introduced artificially to the locus.

The present invention provides a method for treating a plant or a plant material against pest infestation comprising applying an effective amount of at least one compound of Formula I as defined herein or an agricultural acceptable salt thereof to the plant, the plant material, or the vicinity of the plant or plant material so as to thereby treat the plant or plant material against pest infestation, wherein the compound of Formula I is (i) produced synthetically, (ii) applied free of fungal material from Pseudozyma aphidis, and/or applied at an amount such that plant or plant material being treated is exposed to an amount of the compound of Formula I that is higher than the amount the plant or plant material would be exposed to from secretions of Pseudozyma aphidis that (a) exist naturally on the plant or plant material, or in the vicinity thereof or (b) is introduced artificially to the plant, plant material, or vicinity thereof.

The present invention provides a method for controlling and/or preventing of fungal attack on soil, plant, root, foliage, seed, locus of the fungus, or a locus where fungal infestation is to be prevented, wherein the method comprises applying a fungicidally effective amount of at least one compound of Formula I as defined herein or an agriculturally acceptable salt thereof to the soil, plant, roots, foliage, seed, locus of the fungus, or locus in which the infestation is to be prevented so as to thereby control and/or prevent fungal attack, wherein the compound of Formula I is (i) produced synthetically, (ii) applied free of fungal material from Pseudozyma aphidis, and/or (iii) applied at an amount such that the soil, plant, root, foliage, seed, locus of the fungus, or locus being treated is exposed to an amount of the compound of Formula I that is higher than the amount the soil, plant, root, foliage, seed, locus of the fungus, or locus would be exposed to from secretions of Pseudozyma aphidis that (a) exist naturally at the soil, plant, root, foliage, seed, locus of the fungus, or locus, or (b) is introduced artificially to the soil, plant, root, foliage, seed, locus of the fungus, or locus.

The present invention provides a composition comprising (i) at least one compound of Formula I as described herein or an agriculturally acceptable salt thereof, and (ii) an agriculturally acceptable carrier, wherein the concentration of the compound of Formula I in the composition is 0.03-1000 g/L.

The present invention provides a combination comprising (i) at least one compound of Formula I as described herein or an agriculturally acceptable salt thereof, and (ii) at least one agriculturally acceptable carrier.

The present invention provides a combination comprising (i) at least one compound of Formula T as described herein or an agriculturally acceptable salt thereof, and (ii) at least one additional pesticide.

The present invention provides a combination comprising (i) at least one composition as described herein, and (ii) at least one additional pesticide.

The present invention provides a method of treating a locus against pest infestation comprising applying an effective amount of any one of the composition or combination described herein to the locus so as to thereby treat the locus against pest infestation.

The present invention provides a method for treating a plant or a plant material against pest infestation comprising applying an effective amount of any one of the composition or combination described herein to the plant, the plant material, or the vicinity of the plant or plant material so as to thereby treat the plant or plant material against pest infestation.

The present invention provides a method for controlling and/or preventing of fungal attack on soil, plant, root, foliage, seed, locus of the fungus, or a locus where fungal infestation is to be prevented, wherein the method comprises applying a fungicidally effective amount of any one of the composition or combination described herein to the soil, plant, roots, foliage, seed, locus of the fungus, or locus in which the infestation is to be prevented so as to thereby control and/or prevent fungal attack.

The present invention provides use of a compound of Formula I as described herein or an agriculturally acceptable salt thereof in the manufacturing of a composition for treating a locus against pest infestation.

The present invention provides use of a compound of Formula I as described herein or an agriculturally acceptable salt thereof in the manufacturing of a composition for treating a plant or a plant material against pest infestation.

The present invention provides use of a compound of Formula I as described herein or an agriculturally acceptable salt thereof in the manufacturing of a composition for controlling and/or preventing of fungal attack on soil, plant, root, foliage, seed, locus of the fungus, or a locus where fungal infestation is to be prevented.

The present invention provides a method of preparing a crude extract comprising a compound of Formula I or agriculturally acceptable salt thereof comprising the steps of:
(i) producing a conditioned culture medium of a fungus,
(ii) providing nitrogen-limiting conditions,
(iii) filtering the medium through a resin, and
(iv) eluting the adsorbed material from the resin with a polar solvent.

The present invention provides a process for extracting the compound of Formula I from a fungus comprising:
(i) producing a conditioned culture medium of the fungus,
(ii) providing nitrogen-limiting conditions,
(iii) filtering the medium through a resin,
(iv) eluting the adsorbed material from the resin with a polar solvent, and
(v) isolating and purifying the compound of Formula (I) from the eluted material.

The present invention also provides a process for preparation of compound of formula (Ia) comprising:

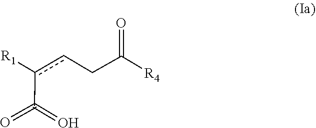

a) reacting a compound of formula (II) with compound of formula (III) in the presence of base and optionally in the presence of organic solvent

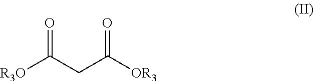

R₁X (III)
to form a compound of formula (IV),

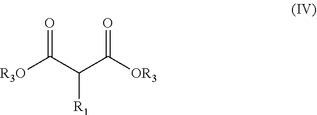

b) reacting the compound of formula (IV) with a compound of formula (V) in the presence of base and optionally in the presence of organic solvent

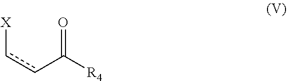

to form a compound of formula (VI), and

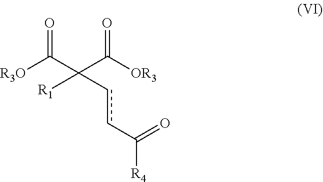

c) decarboxylating the compound of formula (VI) in the presence of catalyst and optionally in the presence of organic solvent to form the compound of formula (Ia),
wherein
R₁ is a C₁-C₁₂ alkyl optionally substituted with one or more substituents independently selected from the group consisting of halogen, C(O)R, CN, NH₂, —NH(C₁-C₄ alkyl), —(C₁-C₄ alkyl)—NH₂, —N(C₂-C₄ alkyl)(C₂-C₄ alkyl), OR, —S(O)₂(C₁-C₄ alkyl), —S(O)(C₁-C₄ alkyl), C(O)OH and C(O)OR;
R₃ is hydrogen or C₁-C₄ alkyl;
R₄ is OH, —O—, —NH₂, —NH—NH₂, —NH(C₁-C₁₂ alkyl), —N(C₂-C₄ alkyl)(C₂-C₄ alkyl), —O(C₁-C₁₂ alkyl), halogen, SR₃, or S(O)R₃;
X is bromine, chlorine or fluorine; and
R is independently H or an optionally substituted C₁-C₁₂ alkyl group.

The present invention also provides a process for preparation of compound of formula (Ia) comprising:

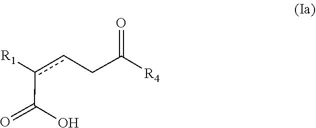

a) reacting a compound of formula (II) with compound of formula (III) in the presence of base and optionally in the presence of organic solvent

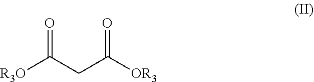

R₁X (III)
to form a compound of formula (V),

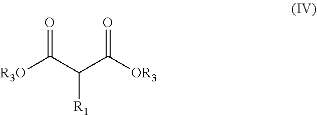

b) reacting the compound of formula (IV) with a compound of formula (V) in the presence of base and optionally in the presence of organic solvent

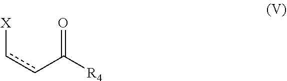

to form a compound of formula (VI), and

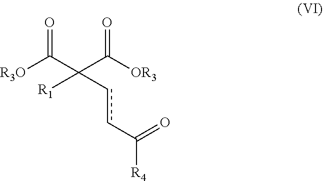

c) decarboxylating the compound of formula (VI) in the presence of catalyst and optionally in the presence of organic solvent to form the compound of formula (Ia),
wherein
R₁ is a C₁-C₁₂ alkyl optionally substituted with one or more substituents independently selected from the group consisting of halogen, CN, NH₂, C₁-C₄ alkylamino, C₂-C₄ dialkylamino, OR, C₁-C₄ alkylsulfonyl, C₁-C₄ alkylsulfinyl, C(O)OH, C(O)OR;
R₃ is hydrogen or C₁-C₄ alkyl;
R₄ is OH, —O⁻, —NH₂, —NH—NH₂, —NH(C₁-C₁₂ alkyl), —N(C₂-C₄ alkyl)(C₂-C₄ alkyl), —O(C₁-C₁₂ alkyl), halogen, SR₃, or S(O)R₃;
X is bromine, chlorine or fluorine; and
R is H or an optionally substituted C₁-C₁₂ alkyl group.

The present invention also provides a process for preparation of compound of formula (Ia) comprising:

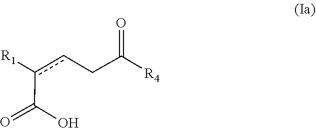

a) reacting a compound of formula (II) with compound of formula (III) in the presence of base and optionally in the presence of organic solvent

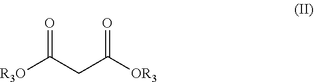

R₁X (III)
to form a compound of formula (IV),

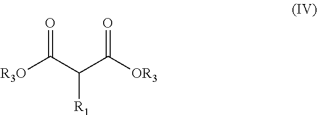

b) reacting the compound of formula (IV) with a compound of formula (V) in the presence of base and optionally in the presence of organic solvent

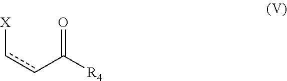

to form a compound of formula (VI), and

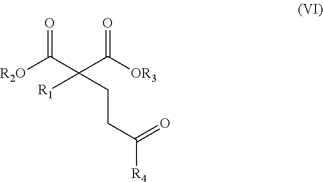

c) decarboxylating the compound of formula (VI) in the presence of catalyst and optionally in the presence of organic solvent to form the compound of formula (Ia),
wherein
R₁ is a C₁-C₁₂ alkyl optionally substituted with one or more substituents independently selected from the group consisting of halogen, CN, NH₂, C₁-C₄ alkylamino, C₂-C₄ dialkylamino, OR, C₁-C₄ alkylsulfonyl, C₁-C₄ alkylsulfinyl, C(O)OH, C(O)OR;
R₃ is hydrogen or C₁-C₄ alkyl;
R₄ is OH, C₁-C₁₂ alkoxy, halogen, C₁-C₁₂ alkyl amino, SR₃, or S(O)R₃;
X is bromine, chlorine or fluorine; and
R is H or an optionally substituted C₁-C₁₂ alkyl group.

### BRIEF DESCRIPTION OF THE FIGURES

FIG. 1 shows the antifungal activity of 2-octyl-2 pentenedioic acid against B. cinerea at different pH.

FIG. 2 shows the antifungal activity of Pseudozyma aphidis crude extract, which comprises 2-octyl-2 pentenedioic acid, against B. cinereal at different pH.

FIGS. 3a and 3b show the in planta fungicidal activity and calculated efficacy of the Pseudozyma aphidis crude extract, which comprises 2-octyl-2 pentenedioic acid, and the synthetic 2-octyl-2 pentenedioic acid against Puccinia triticina.

FIGS. 4a and 4b show the in planta fungicidal activity and calculated efficacy of 2-octyl-2 pentenedioic acid extracted from Pseudozyma aphidis (PX1—in Methanol), against Puccinia. triticina.

FIGS. 5a and 5b show the fungicidal activity and calculated efficacy of the crude extract of P. aphidis applied at 1% against Puccinia triticina compared to Rhapsody® (applied at 2.0 L/ha) and to Amistar® (applied at 1 L/ha).

FIGS. 6a and 6b show the in planta fungicidal activity of synthetic 2-octyl-2 pentenedioic acid (PA-X1) against Zymoseptoria tritici.

FIGS. 7a and 7b show the in planta fungicidal activity and calculated efficacy of 2-octyl-2 pentenedioic acid extracted from Pseudozyma aphidis (PX1—in Methanol), against Zymoseptoria tritici.

FIGS. 8a and 8b show the fungicidal effect and calculated efficacy of the crude extract (YE) of P. aphidis at 1%, Amylo-x® (Bacillus amyloliquefasciens) and Proline® (prothioconazole) on the intensity of infection on wheat leaf fragments cv. ALIXAN by Zymoseptoria tritici strain Mg Tri-R6.

FIGS. 9a and 9b show the in planta fungicidal activity and calculated efficacy of synthetic 2-octyl-2 pentenedioic acid (PA-X1) against Phytophthora infestans.

FIGS. 10a and 10b show the in planta fungicidal activity and calculated efficacy of 2-octyl-2 pentenedioic acid in methanol against Phytophthora infestans.

FIGS. 11a and 11b show the fungicidal effect and calculated efficacy of the crude extract (YE) of P. aphidis, LBG 01F34® (potassium phosphonates) and Revus® (mandipropamid) on the intensity of infection on tomato plants var. microtome by Phytophthora infestans strain Pi61.

FIG. 12 shows the activity of P. aphidis extracts/eluates differ by C:N feeding ratio during production against B. cinerea using the bioassay in a liquid medium.

FIG. 13 shows the activity of P. aphidis extracts differ by C:N feeding ratio during production with amberlite resin against B. cinerea using agar plates assay.

FIG. 14 shows the efficacy of the extract obtained using various extraction methods of P. aphidis against B. cinerea using the bio-assay with tomato leaves.

### DETAILED DESCRIPTION OF SPECIFIC EMBODIMENTS OF THE INVENTION

The Applicant has surprisingly found that compound of Formula I:

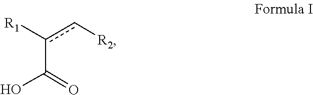

wherein
R₁ is a C₁-C₁₂ alkyl optionally substituted with one or more substituents independently selected from the group consisting of halogen, C(O)R, CN, NH₂, —NH(C₁-C₄ alkyl), —(C₁-C₄ alkyl)—NH₂, —N(C₂-C₄ alkyl)(C₂-C₄ alkyl), OR, —S(O)₂(C₁-C₄ alkyl), —S(O)(C₁-C₄ alkyl), C(O)OH and C(O)OR,
R₂ is —(C₁-C₁₂)—COOH, —(C₁-C₁₂)—COO, —(C₁-C₁₂)—COOR, —(C₁-C₁₂)—CONH₂, —(C₁-C₁₂)—CONHR, —(C₁-C₁₂)—CONR₂, —(C₁-C₁₂)—COSR, —(C₁-C₁₂)—COX, —(C₁-C₁₂)—S(O)₂NH₂, —(C₁-C₁₂)—S(O)₂NHR, —(C₁-C₁₂)—S(O)₂NR₂, —(C₁-C₁₂)—CN, —(C₁-C₁₂)—CR═NR, —(C₁-C₁₂)—CONHNH₂, wherein each of the —(C₁-C₁₂)—COOH, —(C₁-C₁₂)—COO⁻, —(C₁-C₁₂)—COOR, —(C₁-C₁₂)—CONH₂, —(C₁-C₁₂)—CONHR, —(C₁-C₁₂)—CONR₂, —(C₁-C₁₂)—COSR, —(C₁-C₁₂)—COX, —(C₁-C₁₂)—S(O)₂NH₂, —(C₁-C₁₂)—S(O)₂NHR, —(C₁-C₂)—S(O)₂NR₂, —(C₁-C₁₂)—CN, —(C₁-C₁₂)—CR═NR, and —(C₁-C₁₂)—CONHNH₂ may be optionally substituted with halogen, amine, carboxylic acid, OR, CN, a C₁-C₁₂ thioester, and/or sulphonyl,
R is independently H or an optionally substituted C₁-C₁₂ alkyl group,
X is halogen, and
[symbol] is an optional double bond;

and agriculturally acceptable salts thereof have unexpected pesticidal activities and have proven to be capable of controlling numerous pests which can damage crops of considerable economical interest. In particular, the compound of Formula I is selected from the group consisting of (E)-2-octyl-2-pentenedioic acid, (Z)-2-octyl-2-pentenedioic acid, and (E/Z)-2-octyl-2-pentenedioic acid.

The compounds of Formula I and agriculturally acceptable salts thereof of the present disclosure may be applied by any of a variety of known techniques, either as compounds or as compositions (including formulations) comprising the compounds or agriculturally acceptable salts thereof. For example, the compounds of Formula I or agriculturally acceptable salts thereof may be applied to the roots, seeds or foliage of plants for the control of various pests including fungi and/or bacteria, without damaging the plants.

The compounds of Formula I and agriculturally acceptable salts thereof are suitable for treatment of various crops. The compounds and agriculturally acceptable salts thereof may be useful both as a protectant, curative and/or an eradicant pesticide. The compounds and agriculturally acceptable salts thereof of the present subject matter have been found to be effective for use with agricultural crops and horticultural plants.

### Definitions

Prior to setting forth the present subject matter in detail, it may be helpful to provide definitions of certain terms to be used herein. Unless defined otherwise, all technical and scientific terms used herein have the meaning commonly understood by one of skill in the art to which this subject matter pertains.

As used herein, the term “locus” includes not only areas where pest infestation may already be shown, but also areas where pest infestation has yet to show, and also areas under cultivation. “Locus” includes, but is not limited to, soil and other plant growth medium, a habitat, breeding ground, plant (including propagation material thereof), crop (including propagation material thereof), area surrounding the plant or crop, crop field, material or environment in which a pest is growing or may grow.

As used herein the term “plant” or “crop” includes reference to whole plants, plant organs (e.g. leaves, stems, twigs, roots, trunks, limbs, shoots, fruits etc.), plant cells, and propagation material of plants. The term “plant” also encompasses plant crops such as fruits.

As used herein, the term “propagation material” when used in connection with plant includes all the generative parts of the plant such as seeds, spores, seedlings, young plants and vegetative structures such as cuttings, bulbs, corms, tubers, rhizomes, roots stems, sprouts, basal shoots, stolons and buds which may be transplanted after germination or after emergence from soil and can be used for the multiplication of the plant.

As used herein, the term “conventional plant” refers to a plant which has been modified by breeding, mutagenesis or genetic engineering.

As used herein, the term “transgenic plant” refers to a plant having genetic material that has been modified by the use of recombinant DNA techniques. Typically, one or more genes have been integrated into the genetic material of such a plant in order to improve certain properties of the plant.

As used herein, the term “treating a locus against pest infestation” includes, but is not limited to, protecting the locus against pest infestation and/or controlling pest infestation affecting the locus.

As used herein, the term “protecting a locus against pest infestation” includes, but is not limited to, preventing pest infestation from affecting the locus, protecting the locus from pest infestation, delaying the onset of pest infestation affecting the locus and/or conferring resistance to the locus against a pest infestation.

As used herein, the term “controlling pest infestation affecting the locus” includes, but is not limited to, combatting the pest infestation affecting the locus, reducing severity of the pest infestation affecting the locus, curing the pest infestation affecting the locus, ameliorating the pest infestation affecting the locus, inhibiting the pest infestation affecting the locus, and/or eliminating the pest infestation affecting the locus.

As used herein, the term “pest” includes, but is not limited to, unwanted fungus, unwanted pathogen, unwanted insect, unwanted nematode, unwanted mite, unwanted ticks, unwanted arthropod, unwanted bacteria, unwanted parasites, unwanted weed.

As used herein, the term “pesticide” broadly refers to an agent that can be used to prevent and/or control (including kill) a pest. The term “pesticide” is understood to include but is not limited to fungicides, insecticides, nematocides, miticides, arthropodicides, bactericides, acaricides, parasiticides, herbicides and other control agents. For chemical classes and applications, as well as specific compounds of each class, see “The Pesticide Manual Thirteenth Edition” (British Crop Protection Council, Hampshire, U K, 2003), as well as “The e-Pesticide Manual, Version 3” (British Crop Protection Council, Hampshire, U K, 2003-04), the contents of each of which are incorporated herein by reference in their entirety.

The term “applying”, as used herein, refers to applying the compounds and/or compositions of the invention to the plant, to a site of infestation by fungi, to a potential site of infestation by the fungi, which may require protection from infestation, or the environment around the habitat or potential habitat of the fungi. The application may be by methods described in the present invention such as by spraying, dipping, seed coating, post-harvest applications, pre-packing applications, etc.

As used herein, the term “effective amount” refers to an amount of the agrochemical composition or of the mixture which is sufficient for controlling harmful fungi on crop plants and does not cause any significant damage to the treated crop plants.

As used herein, the term “combination” means an assemblage of agrochemicals for application either by simultaneous or contemporaneous application.

As used herein, the term “simultaneous” when used in connection with application of agrochemicals means that the agrochemicals are applied in an admixture, for example, a tank mix. For simultaneous application, the combination may be the admixture or separate containers each containing an agrochemical that are combined prior to application.

As used herein, the term “contemporaneous” when used in connection with application of agrochemicals means that an individual agrochemical is applied separately from another agrochemical or premixture at the same time or at times sufficiently close together that an activity that is additive or more than additive or synergistic relative to the activity of either agrochemical alone at the same dose is achieved.

As used herein, the term “mixture” refers, but is not limited to, a combination in any physical form, e.g., blend, solution, alloy, or the like.

As used herein, the term “tank mix” means one or more of the components of the combination, mixture or composition of the present invention are added are mixed in a spray tank at the time of spray application or prior to spray application.

As used herein, the term “composition” includes at least one of the combinations or mixtures of the present invention with an agriculturally acceptable carrier. The composition may be a formulation, including commercial formulation.

The term “plant health” comprises various sorts of improvements of plants that are not connected to the control of pests. For example, advantageous properties that may be mentioned are improved crop characteristics including: emergence, crop yields, protein content, oil content, starch content, more developed root system (improved root growth), improved stress tolerance (e.g. against drought, heat, salt, UV, water, cold), reduced ethylene (reduced production and/or inhibition of reception), increase in plant height, bigger leaf blade, less dead basal leaves, stronger tillers, greener leaf color, pigment content, photosynthetic activity, less input needed (such as fertilizers or water), less seeds needed, more productive tillers, earlier flowering, early grain maturity, less plant verse (lodging), increased shoot growth, enhanced plant vigor, increased plant stand and early and better germination; or any other advantages familiar to a person skilled in the art.

As used herein, the term “effective” when used in connection with an amount of the active ingredient, combination, mixture or composition refers to an amount of the active ingredient, combination, mixture or composition that achieve an agriculturally beneficial level of control of the fungus, pathogen, and/or disease when applied to a plant, propagation material of the plant, soil or a locus.

As used herein, the term “disease-inhibiting and phytologically acceptable amount” refers to an amount of a compound that kills or inhibits the plant disease for which control is desired but is not significantly toxic to the plant.

As used herein, the term “agriculturally acceptable carrier” means carriers which are known and accepted in the art for the formation of compositions for agricultural or horticultural use.

As used herein, the term “fungal material” means any material, other than compound(s) of Formula I, that is contained in a fungal cell, generated in a fungal cell, and/or secreted by a fungal cell. Examples of fungal material include spores and culture filtrate metabolites. Fungal material may be found in, for example, extracts or conditioned medium.

As used herein, the term “conditioned medium” means a medium or a liquid carrier in which the fungus was previously grown and to which the fungus secreted compounds.

As used herein, the term “culture filtrate metabolites” when used in connection with a fungus refer to compounds secreted by the fungus into the growth media.

As used herein, the term “extract” refers to any substance obtained by extracting fungal cells, spores, culture filtrate or conditioned medium using organic solvents.

As used herein, a compound “produced synthetically” or a “synthetic” compound refers to a compound that is not produced by a living organism by way of biological process(es). A composition comprising the compound produced synthetically or the synthetic compound may comprise impurities as a result of the synthesis process used.

As used herein, a “derivative from Pseudozyma aphidis” means (a) a Pseudozyma aphidis cell or an isolate or mutant thereof, (b) a Pseudozyma aphidis spore, (c) a conditioned culture medium of Pseudozyma aphidis, (d) secretions from Pseudozyma aphidis, (e)extracts of any of(a) to (d), or a combination of at least two of the biocontrol agents defined in (a) to (e). Secretions from Pseudozyma aphidis includes fractions of the secretions. Conditioned culture medium includes factions of conditioned culture medium.

The terms “Pseudozyma aphidis cell”, “Pseudozyma aphidis spore”, “conditioned culture medium of Pseudozyma aphidis”, “secretion from Pseudozyma aphidis”, “isolate”, “mutant”, “extract”, “fraction”, and “biocontrol agent” shall each have the same meaning as the meaning the term has in U.S. Pat. No. 8,975,213, issued Mar. 10, 2015, and U.S. Pat. No. 9,161,545, issued Oct. 20, 2015.

As used herein, the term “a composition comprising a derivative from Pseudozyma aphidis” refers to a composition comprising any one or any combination of derivative(s) from Pseudozyma aphidis as described in U.S. Pat. No. 8,975,213, issued Mar. 10, 2015, and U.S. Pat. No. 9,161,545, issued Oct. 20, 2015.

As used herein, the term “infestation” includes the presence of pest in or on any locus that causes damage or disease.

As used herein the term “ha” refers to hectare.

Throughout the application, descriptions of various embodiments use the term “comprising”; however, it will be understood by one of skill in the art, that in some specific instances, an embodiment can alternatively be described using the language “consisting essentially of” or “consisting of.”

The term “a” or “an” as used herein includes the singular and the plural, unless specifically stated otherwise. Therefore, the terms “a,” “an” or “at least one” can be used interchangeably in this application.

The term “alkyl” as used herein refers to a branched, unbranched, or cyclic carbon chain.

The term “halogen” or “halo” as used herein refers to one or more halogen atoms, defined as F, Cl, Br, and I.

Unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques. In an embodiment, use of the term “about” herein specifically includes ±10% from the indicated values in the range. In addition, the endpoints of all ranges directed to the same component or property herein are inclusive of the endpoints, are independently combinable, and include all intermediate points and ranges. Where a range is given in the specification it is understood that the range includes all integers and 0.1 units within that range, and any sub-range thereof. For example, a range of “10 g/ha to 10000 g/ha” is a disclosure of 10.1 g/ha, 10.2 g/ha, 10.3 g/ha, 10.4 g/ha etc. up to 10000 g/ha.

The present invention provides a method of treating a locus against pest infestation comprising applying an effective amount of at least one compound of Formula I comprising applying an effective amount of at least one compound of Formula I:

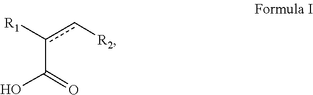

wherein
R₁ is a C₁-C₁₂ alkyl optionally substituted with one or more substituents independently selected from the group consisting of halogen, C(O)R, CN, NH₂, —NH(C₁-C₄ alkyl), —(C₁-C₄ alkyl)—NH₂, —N(C₂-C₄ alkyl)(C₂-C₄ alkyl), OR, —S(O)₂(C₁-C₄ alkyl), —S(O)(C₁-C₄ alkyl), C(O)OH and C(O)OR,
R₂ is —(C₁-C₁₂)—COOH, —(C₁-C₁₂)—COO, —(C₁-C₁₂)—COOR, —(C₁-C₁₂)—CONH₂, —(C₁-C₁₂)—CONHR, —(C₁-C₁₂)—CONR₂, —(C₁-C₂)—COSR, —(C₁-C₁₂)—COX, —(C₁-C₁₂)—S(O)₂NH₂, —(C₁-C₁₂)—S(O)₂NHR, —(C₁-C₁₂)—S(O)₂NR₂, —(C₁-C₁₂)—CN, —(C₁-C₂)—CR═NR, —(C₁-C₁₂)—CONHNH₂, wherein each of the —(C₁-C₂)—COOH, —(C₁-C₁₂)—COO—, —(C₁-C₁₂)—COOR, —(C₁-C₁₂)—CONH₂, —(C₁-C₁₂)—CONHR, —(C₁-C₁₂)—CONR₂, —(C₁-C₁₂)—COSR, —(C₁-C₂)—COX, —(C₁-C₁₂)—S(O)₂NH₂, —(C₁-C₁₂)—S(O)₂NHR, —(C₁-C₁₂)—S(O)₂NR₂, —(C₁-C₁₂)—CN, —(C₁-C₁₂)—CR═NR, and —(C₁-C₁₂)—CONHNH₂ may be optionally substituted with halogen, amine, carboxylic acid, OR, CN, a C₁-C₂ thioester, and/or sulphonyl,
R is independently H or an optionally substituted C₁-C₁₂ alkyl group,
X is halogen, and
[symbol] is an optional double bond;

or an agriculturally acceptable salt thereof,

to the locus so as to thereby treat the locus against pest infestation, wherein the compound of Formula I is applied free of fungal material from Pseudozyma aphidis.

The present invention provides a method of treating a locus against pest infestation comprising applying an effective amount of at least one compound of Formula I comprising applying an effective amount of at least one compound of Formula I:

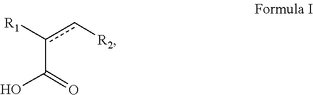

wherein
R₁ is a C₁-C₁₂ alkyl optionally substituted with one or more substituents independently selected from the group consisting of halogen, CN, NH₂, —NH(C₁-C₄ alkyl), —(C₁-C₄ alkyl)—NH₂, —N(C₂-C₄ alkyl)(C₂-C₄ alkyl), OR, —S(O)₂(C₁-C₄ alkyl), —S(O)(C₁-C₄ alkyl), C(O)OH and C(O)OR,
R₂ is —(C₁-C₁₂)—COOH, —(C₁-C₁₂)—CONH₂, —(C₁-C₁₂)—CONHR, —(C₁-C₁₂)—CONR₂, —(C₁-C₁₂)—COSR, —(C₁-C₂)—COX, —(C₁-C₁₂)—S(O)₂NH₂, —(C₁-C₁₂)—S(O)₂NHR or —(C₁-C₁₂)—S(O)₂NR₂, wherein each of the —(C₁-C₁₂)—COOH, —(C₁-C₁₂)—CONH₂, —(C₁-C₁₂)—CONHR, —(C₁-C₁₂)—CONR₂, —(C₁-C₁₂)—COSR, —(C₁-C₁₂)—COX, —(C₁-C₁₂)—S(O)₂NH₂, —(C₁-C₁₂)—S(O)₂NHR and —(C₁-C₁₂)—S(O)₂NR₂ may be optionally substituted with halogen, amine, carboxylic acid, OR, CN, a C₁-C₁₂ thioester, and/or sulphonyl,
R is independently H or an optionally substituted C₁-C₁₂ alkyl group,
X is halogen, and
[symbol] is an optional double bond;

or an agriculturally acceptable salt thereof,

to the locus so as to thereby treat the locus against pest infestation, wherein the compound of Formula I is applied free of fungal material from Pseudozyma aphidis.

The present invention provides a method of treating a locus against pest infestation comprising applying an effective amount of at least one compound of Formula I comprising applying an effective amount of at least one compound of Formula I:

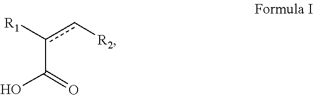

wherein
R₁ is a C₁-C₁₂ alkyl optionally substituted with one or more substituents independently selected from the group consisting of halogen, CN, NH₂, C₁-C₄ alkylamino, C₂-C₄ dialkylamino, OR, C₁-C₄ alkylsulfonyl, C₁-C₄ alkylsulfinyl, C(O)OH, C(O)OR,
R₂ is a C₁-C₁₂ (a) carboxylic acid, (b) amide, (c) thioester, (d) acyl halide, or (e) sulfonyl amide, wherein each of the C₁-C₁₂ carboxylic acid, amid, thioester, acyl halide and sulfonyl amide may be optionally substituted with halogen, amine, carboxylic acid, OR, CN, a C₁-C₁₂ thioester, and/or sulphonyl,
R is H or an optionally substituted C₁-C₁₂ alkyl group, and
[symbol] is an optional double bond;
or an agriculturally acceptable salt thereof,

to the locus so as to thereby treat the locus against pest infestation, wherein the compound of Formula I is applied free of fungal material from Pseudozyma aphidis.

In some embodiments, the compound of Formula I is applied in acid form.

For example, 2-octyl-2-pentenedioic acid is a compound of Formula I. 2-octyl-2-pentenedioic acid may exist in dimer or polymer form as shown below.

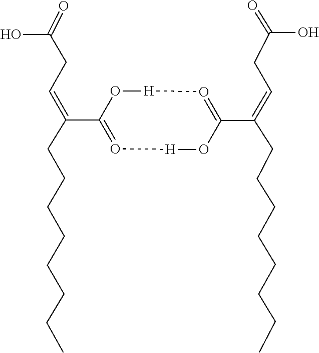

The present invention provides a method of treating a locus against pest infestation comprising applying an effective amount of at least one compound of Formula I comprising applying an effective amount of at least one compound of Formula I:

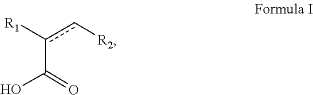

wherein
R₁ is a C₁-C₁₂ alkyl optionally substituted with one or more substituents independently selected from the group consisting of halogen, C(O)R, CN, NH₂, —NH(C₁-C₄ alkyl), —(C₁-C₄ alkyl)—NH₂, —N(C₂-C₄ alkyl)(C₂-C₄ alkyl), OR, —S(O)₂(C₁-C₄ alkyl), —S(O)(C₁-C₄ alkyl), C(O)OH and C(O)OR,
R₂ is —(C₁-C₁₂)—COOH, —(C₁-C₁₂)—COO⁻,—(C₁-C₁₂)—COOR, —(C₁-C₁₂)—CONH₂, —(C₁-C₁₂)—CONHR, —(C₁-C₁₂)—CONR₂, —(C₁-C₁₂)—COSR, —(C₁-C₁₂)—COX, —(C₁-C₁₂)—S(O)₂NH₂, —(C₁-C₁₂)—S(O)₂NHR, —(C₁-C₁₂)—S(O)₂NR₂, —(C₁-C₁₂)—CN, —(C₁-C₁)—CR═NR, —(C₁-C₁₂)—CONHNH₂, wherein each of the —(C₁-C₁₂)—COOH, —(C₁-C₁₂)—COO—, —(C₁-C₁₂)—COOR, —(C₁-C₁₂)—CONH₂, —(C₁-C₁₂)—CONHR, —(C₁-C₁₂)—CONR₂, —(C₁-C₁₂)—COSR, —(C₁-C₂)—COX, —(C₁-C₁₂)—S(O)₂NH₂, —(C₁-C₁₂)—S(O)₂NHR, —(C₁-C₁₂)—S(O)₂NR₂, —(C₁-C₁₂)—CN, —(C₁-C₁₂)—CR═NR, and —(C₁-C₁₂)—CONHNH₂ may be optionally substituted with halogen, amine, carboxylic acid, OR, CN, a C₁-C₁₂ thioester, and/or sulphonyl,
R is independently H or an optionally substituted C₁-C₁₂ alkyl group,
X is halogen, and
[symbol] is an optional double bond;

or an agriculturally acceptable salt thereof,

to the locus so as to thereby treat the locus against pest infestation, wherein the compound of Formula I is produced synthetically.

The present invention provides a method of treating a locus against pest infestation comprising applying an effective amount of at least one compound of Formula I comprising applying an effective amount of at least one compound of Formula I:

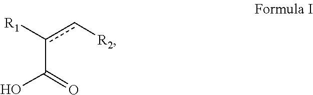

wherein
R₁ is a C₁-C₁₂ alkyl optionally substituted with one or more substituents independently selected from the group consisting of halogen, CN, NH₂, —NH(C₁-C₄ alkyl), —(C₁-C₄ alkyl)—NH₂, —N(C₂-C₄ alkyl)(C₂-C₄ alkyl), OR, —S(O)₂(C₁-C₄ alkyl), —S(O)(C₁-C₄ alkyl), C(O)OH and C(O)OR,
R₂ is —(C₁-C₁₂)—COOH, —(C₁-C₂)—CONH₂, —(C₁-C₁₂)—CONHR, —(C₁-C₁₂)—CONR₂, —(C₁-C₂)—COSR, —(C₁-C₁₂)—COX, —(C₁-C₁₂)—S(O)₂NH₂, —(C₁-C₁₂)—S(O)₂NHR or —(C₁-C₁₂)—S(O)₂NR₂, wherein each of the —(C₁-C₁₂)—COOH, —(C₁-C₁₂)—CONH₂, —(C₁-C₁₂)—CONHR, —(C₁-C₁₂)—CONR₂, —(C₁-C₁₂)—COSR, —(C₁-C₁₂)—COX, —(C₁-C₁₂)—S(O)₂NH₂, —(C₁-C₁₂)—S(O)₂NHR and —(C₁-C₁₂)—S(O)₂NR₂ may be optionally substituted with halogen, amine, carboxylic acid, OR, CN, a C₁-C₁₂ thioester, and/or sulphonyl,
R is independently H or an optionally substituted C₁-C₁₂ alkyl group,
X is halogen, and
[symbol] is an optional double bond;

or an agriculturally acceptable salt thereof,

to the locus so as to thereby treat the locus against pest infestation, wherein the compound of Formula I is produced synthetically.

The present invention provides a method of treating a locus against pest infestation comprising applying an effective amount of at least one compound of Formula I comprising applying an effective amount of at least one compound of Formula I:

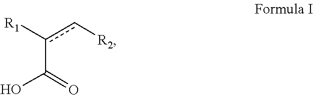

wherein
R₁ is a C₁-C₁₂ alkyl optionally substituted with one or more substituents independently selected from the group consisting of halogen, CN, NH₂, C₁-C₄ alkylamino, C₂-C₄ dialkylamino, OR, C₁-C₄ alkylsulfonyl, C₁-C₄ alkylsulfinyl, C(O)OH, and C(O)OR,
R₂ is a C₁-C₁₂ (a) carboxylic acid, (b) amide, (c) thioester, (d) acyl halide, or (e) sulfonyl amide, wherein each of the C₁-C₁₂ carboxylic acid, amid, thioester, acyl halide and sulfonyl amide may be optionally substituted with halogen, amine, carboxylic acid, OR, CN, a C₁-C₁₂ thioester, and/or sulphonyl,
R is H or an optionally substituted C₁-C₁₂ alkyl group, and
[symbol] is an optional double bond;
or an agriculturally acceptable salt thereof,

to the locus so as to thereby treat the locus against pest infestation, wherein the compound of Formula I is produced synthetically.

In some embodiments, the agriculturally acceptable salt is a carboxylic acid salt. In some embodiments, the agriculturally acceptable salt is a carboxylic acid salt at R₂.

In some embodiments, the carboxylic acid is an ester. In some embodiments, the carboxylic acid at R₂ is ester.

In some embodiments, the compound of Formula I includes at least one carboxylic acid in either R₁ or R₂. The compound of Formula I may be applied in acid form. The compound of Formula I may also be applied in the form of disodium salt derivative or other agriculturally acceptable salt derivatives which at the target site (for example, buffer solution within the target cell) may convert to the carboxylic acid form.

In some embodiments, the compound of Formula I is applied in acid form. Acid form of the compound of Formula I refers to the compound of Formula I having at least one carboxylic acid.

In some embodiments, the compound of Formula I is applied in salt form.

In some embodiments, the compound of Formula I is applied as a mixture of acid form and salt form.

In some embodiments, the compound of Formula I is produced synthetically and applied free of fungal material from Pseudozyma aphidis.

In some embodiments, R₁ is a C₆-C₁₂ alkyl.

In some embodiments, R₁ is an octyl.

In some embodiments, R₁ is a C₁-C₁₂ alkyl substituted with C(O)OH. In some embodiments, R₂ is a C₁-C₁₂ carboxylic acid. In some embodiments, R₂ is —(C₁-C₁₂)—COOH. In some embodiments, R₂ is CH₂COOH. In some embodiment, the compound of Formula I is selected from the group consisting of (E)-2-octyl-2-pentenedioic acid, (Z)-2-octyl-2-pentenedioic acid, and (E/Z)-2-octyl-2-pentenedioic acid.

In some embodiments, R₂ is —(C₁-C₁₂)—COO⁻.

In some embodiments, R₂ is —(C₁-C₁₂)—COOR.

In some embodiments, the compound of Formula I is (E)-2-octyl-2-pentenedioic acid.

In some embodiments, the compound of Formula I is (Z)-2-octyl-2-pentenedioic acid.

In some embodiments, the compound of Formula I is (E/Z)-2-octyl-2-pentenedioic acid.

In some embodiments, the compound of Formula I is extracted from a fungus. In some embodiments, the compound of Formula I is extracted from Pseudozyma aphidis. Methods for extracting the compound of Formula I from Pseudozyma aphidis are described herein below.

In some embodiments, the compound of Formula I is isolated from the extract prior to application.

In some embodiments, the compound of Formula I is purified from the extract prior to application.

In some embodiments, the compound of Formula I is applied free of fungal material.

In some embodiments, the compound of Formula I is synthetic.

In some embodiments, the agriculturally acceptable salt of the compound of Formula I is an alkali metal salts. In some embodiments, the alkali metal salt is a lithium salt. In some embodiments, the alkali metal salt is a sodium salt. In some embodiments, the alkali metal salt is potassium salt. In some embodiments, the agriculturally acceptable salt of the compound of Formula I is an alkali or alkaline earth metal salts such as sodium, potassium, lithium, magnesium, calcium, salts of other metals as copper, iron, zinc, cobalt or nickel. In some embodiments, the salt contains ammonium group. In some embodiments the salt contains substituted ammonium salts of any kind.

In some embodiment, the method comprises applying one compound of Formula I or an agriculturally acceptable salt thereof. In some embodiment, the method comprises applying a mixture of two or more compounds of Formula I or agriculturally acceptable salts thereof. In some embodiments, the method comprises applying a racemic mixture of 2-octyl-2-pentenedioic acid.

In some embodiments, treating the plant or locus against pest infestation is protecting the locus against pest infestation. In some embodiments, protecting the locus against pest infestation is preventing pest infestation from affecting the locus. In some embodiments, protecting the locus against pest infestation is protecting the locus from pest infestation. In some embodiments, protecting the locus against pest infestation is delaying the onset of pest infestation affecting the locus. In some embodiments, protecting the locus against pest infestation conferring resistance to the locus against a pest infestation.

In some embodiments, treating the locus against pest infestation is controlling pest infestation affecting the locus. In some embodiments, controlling pest infestation affecting the locus is combatting the pest infestation affecting the locus. In some embodiments, controlling pest infestation affecting the locus is reducing severity of the pest infestation affecting the locus. In some embodiments, controlling pest infestation affecting the locus is curing the pest infestation affecting the locus. In some embodiments, controlling pest infestation affecting the locus is ameliorating the pest infestation affecting the locus. In some embodiments, controlling pest infestation affecting the locus is inhibiting the pest infestation affecting the locus. In some embodiments, controlling pest infestation affecting the locus is eliminating the pest infestation affecting the locus.

In some embodiments, the locus is a plant. In some embodiments, the locus is propagation material of a plant. In some embodiments, the locus is the vicinity of a plant. In some embodiments, the locus is the vicinity of a propagation material of a plant. In some embodiments, the locus is soil. In some embodiments, the locus is foliage of the plant. In some embodiments, the locus is seed of the plant.

In some embodiments, the plant is a conventional plant.

In some embodiments, the plant is a transgenic plant.

In some embodiments, the plant is a crop.

In some embodiments, the method is effective for extending the shelf-life of the produce.

In some embodiments, the method is effective for increasing the storage time of the produce.

As used herein, the term “produce” refers to harvested plant including, but not limited to, fruits and vegetables.

In some embodiments, the plant is a produce. In some embodiments, the plant is a farm produce. In some embodiments, the plant is an industrial produce.

In some embodiments, the method of the invention is effective for preventing, ameliorating, inhibiting, eliminating and/or delaying the onset of pest infection in farm and industrial produce thereby extending the shelf-life or storage time of said produce.

In some embodiments, the pest is a fungus. In some embodiments, the pest infestation is a fungal infestation. In some embodiments, the fungal infestation is caused by at least one of Botrytis cinereal, Zymoseptoria tritici, Phytophthora infestans and Puccinia triticina. In some embodiments, the fungal infestation is caused by Botrytis cinereal. In some embodiments, the fungal infestation is caused by Zymoseptoria tritci. In some embodiments, the fungal infestation is caused by Phytophthora infestans. In some embodiments, the fungal infestation is caused by Puccinia triticina.

In some embodiments, the pest is a bacterium. In some embodiments, the pest infestation is a bacterial infestation. In some embodiments, the bacterial infestation is caused by at least one of Clavibacter michiganensis, Agrobacterium tumefaciens, Xanthomonas campestris p.v. phaseoli, Erwinia amylovora, Pseudomonas syringae pv. lachrymans, Pseudomonas syringae pv. tomato, Streptomyces scabies, Xanthomonas campestris pv. campestris and Xanthomonas capestris pv. Vesicatoria.

In some embodiments, the compound of Formula I or agriculturally acceptable salt thereof is applied to the plant. In some embodiments, the compound of Formula I or agriculturally acceptable salt thereof is applied to propagation material of a plant. In some embodiments, the compound of Formula I or agriculturally acceptable salt thereof is applied to the vicinity of a plant. In some embodiments, the compound of Formula I or agriculturally acceptable salt thereof is applied to the vicinity of a propagation material of a plant. In some embodiments, the compound of Formula I or agriculturally acceptable salt thereof is applied to soil. In some embodiments, application of the compound of Formula I or agriculturally acceptable salt thereof to soil is effective to control pest(s) that are resident there. In some embodiments, the compound of Formula I or agriculturally acceptable salt thereof is applied to seed of the plant. In some embodiments, the compound of Formula I or agriculturally acceptable salt thereof is applied to foliage of the plant.

In some embodiments, the compound of Formula I or agriculturally acceptable salt thereof is applied in the early stages of the crop cycle. In some embodiments, the compound of Formula I or agriculturally acceptable salt thereof is applied pre-sowing of the crop. In some embodiments, the compound of Formula I or agriculturally acceptable salt thereof is applied post-sowing of the crop.

In some embodiments, the compound of Formula I or agriculturally acceptable salt thereof is applied to harvested produce. In some embodiments, application of the compound of Formula I or agriculturally acceptable salt thereof to harvested produce is effective to prevent storage losses.

The effective application rates of the compounds of Formula I or agriculturally acceptable salt thereof cannot generally be defined, as it varies depending upon various conditions such as the type of the formulation, weather conditions, the type of crop and the type of pests.

In some embodiments, the compound of Formula I or agriculturally acceptable salt thereof is combined with one or more other pesticides. In some embodiments, the compound of Formula I or agriculturally acceptable salt thereof is applied simultaneously or contemporaneously with one or more other pesticides. In some embodiments, the compound of Formula I or agriculturally acceptable salt thereof is formulated with one or more other pesticides. Pesticides that may be combined, formulated and/or applied with the compound of Formula I or agriculturally acceptable salt thereof is described herein below. When the compound of Formula I or agriculturally acceptable salt thereof is combined, formulated and/or applied with one or more other pesticides, the application rates of the compound of Formula I or agriculturally acceptable salt thereof and the other active agent(s) may vary depending on the desired effect.

In some embodiments, the compound of Formula I or agriculturally acceptable salt thereof is applied at a rate from 1 g/ha to 10000 g/ha. In some embodiments, the compound of Formula I or agriculturally acceptable salt thereof is applied at a rate from 2 g/ha to 10000 g/ha. In some embodiments, the compound of Formula I or agriculturally acceptable salt thereof is applied at a rate from 2.8 g/ha to 10000 g/ha. In some embodiments, the compound of Formula I or agriculturally acceptable salt thereof is applied at a rate from 3 g/ha to 10000 g/ha. In some embodiments, the compound of Formula I or agriculturally acceptable salt thereof is applied at a rate from 5 g/ha to 10000 g/ha. In some embodiments, the compound of Formula I or agriculturally acceptable salt thereof is applied at a rate from 10 g/ha to 10000 g/ha. In some embodiments, the compound of Formula I or agriculturally acceptable salt thereof is applied at a rate from 50 to 5000 g/ha. In some embodiments, the compound of Formula I or agriculturally acceptable salt thereof is applied at a rate from 100 to 2000 g/ha. In some embodiments, the compound of Formula I or agriculturally acceptable salt thereof is applied at a rate from 1 to 10 g/ha. In some embodiments, the compound of Formula I or agriculturally acceptable salt thereof is applied at a rate from 2 to 10 g/ha. In some embodiments, the compound of Formula I or agriculturally acceptable salt thereof is applied at a rate from 3 to 10 g/ha. In some embodiments, the compound of Formula I or agriculturally acceptable salt thereof is applied at a rate from 10 to 50 g/ha. In some embodiments, the compound of Formula I or agriculturally acceptable salt thereof is applied at a rate from 50 to 100 g/ha. In some embodiments, the compound of Formula I or agriculturally acceptable salt thereof is applied at a rate from 100 to 500 g/ha. In some embodiments, the compound of Formula I or agriculturally acceptable salt thereof is applied at a rate from 500 to 1000 g/ha. In some embodiments, the compound of Formula I or agriculturally acceptable salt thereof is applied at a rate from 1000 to 2000 g/ha. In some embodiments, the compound of Formula I or agriculturally acceptable salt thereof is applied at a rate from 2000 to 3000 g/ha. In some embodiments, the compound of Formula I or agriculturally acceptable salt thereof is applied at a rate from 3000 to 4000 g/ha. In some embodiments, the compound of Formula I or agriculturally acceptable salt thereof is applied at a rate from 4000 to 5000 g/ha.

In some embodiments, the compound of Formula I or agriculturally acceptable salt thereof is applied at a rate greater than 1.4 g/ha. In some embodiments, the compound of Formula I or agriculturally acceptable salt thereof is applied at a rate from 1.4 g/ha to 10000 g/ha. In some embodiments, the compound of Formula I or agriculturally acceptable salt thereof is applied at a rate from 1.4 g/ha to 1000 g/ha. In some embodiments, the compound of Formula I or agriculturally acceptable salt thereof is applied at a rate from 1.4 g/ha to 500 g/ha. In some embodiments, the compound of Formula I or agriculturally acceptable salt thereof is applied at a rate from 1.4 g/ha to 350 g/ha.

In some embodiments, the compound of Formula I or agriculturally acceptable salt thereof is applied at a rate greater than 2.8 g/ha. In some embodiments, the compound of Formula I or agriculturally acceptable salt thereof is applied at a rate from 2.8 g/ha to 10000 g/ha. In some embodiments, the compound of Formula I or agriculturally acceptable salt thereof is applied at a rate from 2.8 g/ha to 1000 g/ha. In some embodiments, the compound of Formula I or agriculturally acceptable salt thereof is applied at a rate from 2.8 g/ha to 500 g/ha. In some embodiments, the compound of Formula I or agriculturally acceptable salt thereof is applied at a rate from 2.8 g/ha to 350 g/ha.

In some embodiments, the compound of Formula I or agriculturally acceptable salt thereof is applied at a rate greater than 5.6 g/ha. In some embodiments, the compound of Formula I or agriculturally acceptable salt thereof is applied at a rate from 5.6 g/ha to 10000 g/ha. In some embodiments, the compound of Formula I or agriculturally acceptable salt thereof is applied at a rate from 5.6 g/ha to 1000 g/ha. In some embodiments, the compound of Formula I or agriculturally acceptable salt thereof is applied at a rate from 5.6 g/ha to 500 g/ha. In some embodiments, the compound of Formula I or agriculturally acceptable salt thereof is applied at a rate from 5.6 g/ha to 350 g/ha.

In some embodiments, the compound of Formula I or agriculturally acceptable salt thereof is applied at a rate from 1.4 g/ha to 350 g/ha. In some embodiments, the compound of Formula Ior agriculturally acceptable salt thereof is applied at a rate from 20 g/ha to 320 g/ha.

In some embodiments, the compound of Formula I or agriculturally acceptable salt thereof is applied at a rate from 1.4 g/ha to 10 g/ha. In some embodiments, the compound of Formula I or agriculturally acceptable salt thereof is applied at a rate from 2.8 g/ha to 10 g/ha. In some embodiments, the compound of Formula I or agriculturally acceptable salt thereof is applied at a rate from 5.6 g/ha to 10 g/ha. In some embodiments, the compound of Formula I or agriculturally acceptable salt thereof is applied at a rate from 10 g/ha to 20 g/ha. In some embodiments, the compound of Formula I or agriculturally acceptable salt thereof is applied at a rate from 20 g/ha to 50 g/ha. In some embodiments, the compound of Formula I or agriculturally acceptable salt thereof is applied at a rate from 50 g/ha to 100 g/ha. In some embodiments, the compound of Formula I or agriculturally acceptable salt thereof is applied at a rate from 100 g/ha to 150 g/ha. In some embodiments, the compound of Formula I or agriculturally acceptable salt thereof is applied at a rate from 150 g/ha to 200 g/ha. In some embodiments, the compound of Formula I or agriculturally acceptable salt thereof is applied at a rate from 200 g/ha to 250 g/ha. In some embodiments, the compound of Formula I or agriculturally acceptable salt thereof is applied at a rate from 250 g/ha to 300 g/ha. In some embodiments, the compound of Formula I or agriculturally acceptable salt thereof is applied at a rate from 300 g/ha to 350 g/ha.

In some embodiments, the compound of Formula I or agriculturally acceptable salt thereof is applied at a rate of 20 g/ha, 40 g/ha, 50 g/ha, 75 g/ha, 80 g/ha, 100 g/ha, 150 g/ha, 160 g/ha, or 320 g/ha.

In some embodiment, the compound of Formula I or agriculturally acceptable salt thereof is applied at a rate between 1 and 5000 ppm. In some embodiment, the compound of Formula I or agriculturally acceptable salt thereof is applied at a rate between 1 and 4000 ppm. In some embodiment, the compound of Formula I or agriculturally acceptable salt thereof is applied at a rate between 1 and 3000 ppm. In some embodiment, the compound of Formula I or agriculturally acceptable salt thereof is applied at a rate between 1 and 2000 ppm. In some embodiment, the compound of Formula I or agriculturally acceptable salt thereof is applied at a rate between 1 and 1000 ppm. In some embodiment, the compound of Formula I or agriculturally acceptable salt thereof is applied at a rate between 10 and 500 ppm.

In some embodiments, the compound of Formula I or agriculturally acceptable salt thereof is applied in a composition comprising crude extract from P. aphidis comprising the compound of Formula I and the compound of Formula I or agriculturally acceptable salt thereof is applied at a rate between 30 and 500 ppm. In some embodiments, the compound of Formula I or agriculturally acceptable salt thereof is applied in a composition comprising crude extract from P. aphidis comprising the compound of Formula I and the compound of Formula I or agriculturally acceptable salt thereof is applied at a rate between 60 and 500 ppm. In some embodiments, the compound of Formula I or agriculturally acceptable salt thereof is applied in a composition comprising crude extract from P. aphidis comprising the compound of Formula I and the compound of Formula I or agriculturally acceptable salt thereof is applied at a rate of 30-100 ppm, 100-150 ppm, 150-200 ppm, 200-250 ppm, 250-300 ppm, 300-350 ppm, 350-400 ppm, or 400-470 ppm.

In some embodiments, the compound of Formula I or agriculturally acceptable salt thereof is (i) synthetic or (ii) purified from the crude extract from P. aphidis comprising the compound of Formula I, and the compound of Formula I or agriculturally acceptable salt thereof is applied at a rate between 10 and 50 ppm. In some embodiments, the compound of Formula I or agriculturally acceptable salt thereof is (i) synthetic or (ii) purified from the crude extract from P. aphidis comprising the compound of Formula I, and the compound of Formula I or agriculturally acceptable salt thereof is applied at a rate between 35 and 45 ppm. In some embodiments, the compound of Formula I or agriculturally acceptable salt thereof is (i) synthetic or (ii) purified from the crude extract from P. aphidis comprising the compound of Formula I, and the compound of Formula I or agriculturally acceptable salt thereof is applied at a rate of 10-20 ppm, 20-30 ppm, 30-40 ppm, or 40-50 ppm.

When the compound of Formula I is applied in combination with another pesticide, the application rate of the compound of Formula I may be adjusted accordingly. In some embodiments, the application rate of the compound of Formula I is lower than the application rates described above.

In some embodiments, the compound of Formula I is applied in a composition comprising synthesized compound of Formula I. In some embodiments, the composition comprising synthesized compound of Formula I has a pH of 7 or less. In some embodiments, the composition comprising synthesized compound of Formula I has a pH of between 2 and 7. In some embodiments, the composition comprising synthesized compound of Formula I has a pH of 6. In some embodiments, the composition comprising synthesized compound of Formula I has a pH of 5. In some embodiments, the composition comprising synthesized compound of Formula I has a pH of 4. In some embodiments, the composition comprising synthesized compound of Formula I has a pH of 3. In some embodiments, the composition comprising synthesized compound of Formula I has a pH of 2.

In some embodiments, the compound of Formula I is applied in a composition comprising purified compound of Formula I. The compound of Formula I may be purified from the crude extract from P. aphidis. In some embodiments, the composition comprising purified compound of Formula I has a pH of 7 or less. In some embodiments, the composition comprising purified compound of Formula I has a pH of between 2 and 7. In some embodiments, the composition comprising purified compound of Formula I has a pH of 6. In some embodiments, the composition comprising purified compound of Formula I has a pH of 5. In some embodiments, the composition comprising purified compound of Formula I has a pH of 4. In some embodiments, the composition comprising purified compound of Formula I has a pH of 3. In some embodiments, the composition comprising purified compound of Formula I has a pH of 2.

In some embodiments, the compound of Formula I is applied in a composition comprising crude extract from P. aphidis comprising the compound of Formula I. The crude extract may be obtained using methods described herein. In some embodiments, the composition comprising the crude extract has a pH of 9 or less. In some embodiments, the composition comprising the crude extract has a pH of between 2 and 9. In some embodiments, the composition comprising the crude extract has a pH of 8.

In some embodiments, the composition comprising the crude extract has a pH of 7. In some embodiments, the composition comprising the crude extract has a pH of 6. In some embodiments, the composition comprising the crude extract has a pH of 5. In some embodiments, the composition comprising the crude extract has a pH of 4. In some embodiments, the composition comprising the crude extract has a pH of 3. In some embodiments, the composition comprising the crude extract has a pH of 2.

The pH as described herein refers to pH in water.

In some embodiments, the compound of Formula I is applied in a composition wherein the composition has a concentration of the compound of Formula I greater than 30 mg/L.

In some embodiments, the compound of Formula I is applied at a rate or an amount such that the locus being treated would be exposed to an amount of the compound of Formula I that is higher than the amount the locus would be exposed to from secretions of Pseudozyma aphidis that exist naturally at the locus or is introduced artificially to the locus, including by way of applying Pseudozyma aphidis spores as in the Examples of U.S. Pat. Nos. 8,975,213 and 9,161,545, and in particular Examples 6-10 and 16.

As used herein, artificially introducing Pseudozyma aphidis to a locus includes any action that causes the locus to have a higher population of Pseudozyma aphidis at a given time than the population of Pseudozyma aphidis that would naturally exists at the locus at the given time.

In some embodiments, the compound of Formula I is applied at a rate or an amount higher than the rate or the amount applied in the Examples of U.S. Pat. Nos. 8,975,213 and 9,161,545, and in particular Examples 6-10 and 16, wherein spore of Pseudozyma aphidis were applied.

In some embodiments, the compound of Formula I is applied at a rate or an amount higher than the rate or the amount that would be applied by applying a culture filtrate faction prepared using the procedure of Example 15 of U.S. Pat. Nos. 8,975,213 and 9,161,545.

In some embodiments, the compound of Formula I is applied at a rate or an amount higher than the rate or the amount that would be applied by applying a concentrated culture filtrate faction prepared using the procedure of Example 15 of U.S. Pat. Nos. 8,975,213 and 9,161,545.

The present invention provides a method of treating a locus against pest infestation comprising applying an effective amount of at least one compound of Formula I comprising applying an effective amount of at least one compound of Formula I:

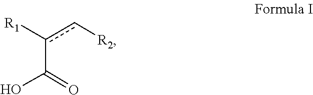

wherein
R₁ is a C₁-C₁₂ alkyl optionally substituted with one or more substituents independently selected from the group consisting of halogen, C(O)R, CN, NH₂, —NH(C₁-C₄ alkyl), —(C₁-C₄ alkyl)—NH₂, —N(C₂-C₄ alkyl)(C₂-C₄ alkyl), OR, —S(O)₂(C₁-C₄ alkyl), —S(O)(C₁-C₄ alkyl), C(O)OH and C(O)OR,
R₂ is —(C₁-C₁₂)—COOH, —(C₁-C₁₂)—COO, —(C₁-C₁₂)—COOR, —(C₁-C₁₂)—CONH₂, —(C₁-C₁₂)—CONHR, —(C₁-C₁₂)—CONR₂, —(C₁-C₁₂)—COSR, —(C₁-C₁₂)—COX, —(C₁-C₁₂)—S(O)₂NH₂, —(C₁-C₁₂)—S(O)₂NHR, —(C₁-C₁₂)—S(O)₂NR₂, —(C₁-C₁₂)—CN, —(C₁-C₁₂)—CR═NR, —(C₁-C₁₂)—CONHNH₂, wherein each of the —(C₁-C₁₂)—COOH, —(C₁-C₁₂)—COO⁻, —(C₁-C₁₂)—COOR, —(C₁-C₁₂)—CONH₂, —(C₁-C₁₂)—CONHR, —(C₁-C₁₂)—CONR₂, —(C₁-C₁₂)—COSR, —(C₁-C₁₂)—COX, —(C₁-C₂)—S(O)₂NH₂, —(C₁-C₁₂)—S(O)₂NHR, —(C₁-C₂)—S(O)₂NR₂, —(C₁-C₁₂)—CN, —(C₁-C₁₂)—CR═NR, and —(C₁-C₁₂)—CONHNH₂ may be optionally substituted with halogen, amine, carboxylic acid, OR, CN, a C₁-C₁₂ thioester, and/or sulphonyl,
R is independently H or an optionally substituted C₁-C₁₂ alkyl group,
X is halogen, and
[symbol] is an optional double bond;

or an agriculturally acceptable salt thereof,

to the locus so as to thereby treat the locus against pest infestation, wherein the compound of Formula I is applied at an amount such that the locus being treated is exposed to an amount of the compound of Formula T that is higher than the amount the locus would be exposed to from secretions of Pseudozyma aphidis that exist naturally at the locus or is introduced artificially to the locus.

The present invention provides a method of treating a locus against pest infestation comprising applying an effective amount of at least one compound of Formula I comprising applying an effective amount of at least one compound of Formula I:

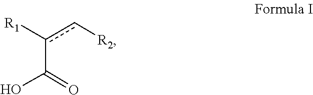

wherein
R₁ is a C₁-C₁₂ alkyl optionally substituted with one or more substituents independently selected from the group consisting of halogen, CN, NH₂, —NH(C₁-C₄ alkyl), —(C₁-C₄ alkyl)—NH₂, —N(C₂-C₄ alkyl)(C₂-C₄ alkyl), OR, —S(O)₂(C₁-C₄ alkyl), —S(O)(C₁-C₄ alkyl), C(O)OH and C(O)OR,
R₂ is —(C₁-C₁₂)—COOH, —(C₁-C₁₂)—CONH₂, —(C₁-C₁₂)—CONHR, —(C₁-C₁₂)—CONR₂, —(C₁-C₁₂)—COSR, —(C₁-C₂)—COX, —(C₁-C₁₂)—S(O)₂NH₂, —(C₁-C₁₂)—S(O)₂NHR or —(C₁-C₁₂)—S(O)₂NR₂,wherein each of the —(C₁-C₁₂)—COOH, —(C₁-C₁₂)—CONH₂, —(C₁-C₁₂)—CONHR, —(C₁-C₁₂)—CONR₂, —(C₁-C₁₂)—COSR, —(C₁-C₁₂)—COX, —(C₁-C₁₂)—S(O)₂NH₂, —(C₁-C₁₂)—S(O)₂NHR and —(C₁-C₁₂)—S(O)₂NR₂ may be optionally substituted with halogen, amine, carboxylic acid, OR, CN, a C₁-C₁₂ thioester, and/or sulphonyl,
R is independently H or an optionally substituted C₁-C₁₂ alkyl group,
X is halogen, and
[symbol] is an optional double bond;

or an agriculturally acceptable salt thereof,

to the locus so as to thereby treat the locus against pest infestation, wherein the compound of Formula I is applied at an amount such that the locus being treated is exposed to an amount of the compound of Formula I that is higher than the amount the locus would be exposed to from secretions of Pseudozyma aphidis that exist naturally at the locus or is introduced artificially to the locus.

The present invention provides a method of treating a locus against pest infestation comprising applying an effective amount of at least one compound of Formula I comprising applying an effective amount of at least one compound of Formula I:

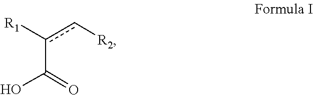

wherein
R¹ is a C₁-C₁₂ alkyl optionally substituted with one or more substituents independently selected from the group consisting of halogen, CN, NH₂, C₁-C₄ alkylamino, C₂-C₄ dialkylamino, OR, C₁-C₄ alkylsulfonyl, C₁-C₄ alkylsulfinyl, C(O)OH, C(O)OR,
R² is a C₁-C₁₂ (a) carboxylic acid, (b) amide, (c) thioester, (d) acyl halide, or (e) sulfonyl amide, wherein each of the C₁-C₁₂ carboxylic acid, amid, thioester, acyl halide and sulfonyl amide may be optionally substituted with halogen, amine, carboxylic acid, OR, CN, a C₁-C₁₂ thioester, and/or sulphonyl,
R is H or an optionally substituted C₁-C₁₂ alkyl group, and
[symbol] is an optional double bond;
or an agriculturally acceptable salt thereof,

to the locus so as to thereby treat the locus against pest infestation, wherein the compound of Formula I is applied at an amount such that the locus being treated is exposed to an amount of the compound of Formula I that is higher than the amount the locus would be exposed to from secretions of Pseudozyma aphidis that exist naturally at the locus or is introduced artificially to the locus.

In some embodiments, the compound of Formula I is applied in acid form.

The compounds of Formula I and agriculturally acceptable salts thereof are effective for use with plants in a disease-inhibiting and agriculturally acceptable amount. This amount will generally be from about 0.1 to about 5000 ppm (parts per million). The exact amount of the compound required varies with the pest to be controlled, the type of formulation employed, the method of application, the particular plant species, climate conditions, and the like. Any range or desired value given herein may be extended or altered without losing the effects sought, as is apparent to the skilled person for an understanding of the teachings herein.

In some embodiments, the compound of Formula I or agriculturally acceptable salt thereof is applied from 1 ppm to 5000 ppm. In some embodiments, the compound of Formula I or agriculturally acceptable salt thereof is applied from 1 ppm to 4000 ppm. In some embodiments, the compound of Formula I or agriculturally acceptable salt thereof is applied from 1 ppm to 3000 ppm. In some embodiments, the compound of Formula I or agriculturally acceptable salt thereof is applied from 1 ppm to 2000 ppm. In some embodiments, the compound of Formula I or agriculturally acceptable salt thereof is applied from 1 ppm to 1000 ppm.

In some embodiments, the compound of Formula I or agriculturally acceptable salt thereof is applied from 1 ppm to 500 ppm. In some embodiments, the compound of Formula I or agriculturally acceptable salt thereof is applied from 1 ppm to 250 ppm. In some embodiments, the compound of Formula I or agriculturally acceptable salt thereof is applied from 1 ppm to 100 ppm. In some embodiments, the compound of Formula I or agriculturally acceptable salt thereof is applied from 1 ppm to 50 ppm. In some embodiments, the compound of Formula I or agriculturally acceptable salt thereof is applied from 1 ppm to 10 ppm. In some embodiments, the compound of Formula I or agriculturally acceptable salt thereof is applied from 10 ppm to 20 ppm. In some embodiments, the compound of Formula I or agriculturally acceptable salt thereof is applied from 20 ppm to 30 ppm. In some embodiments, the compound of Formula I or agriculturally acceptable salt thereof is applied from 30 ppm to 40 ppm. In some embodiments, the compound of Formula I or agriculturally acceptable salt thereof is applied from 40 ppm to 50 ppm. In some embodiments, the compound of Formula I or agriculturally acceptable salt thereof is applied from 50 ppm to 60 ppm. In some embodiments, the compound of Formula I or agriculturally acceptable salt thereof is applied from 60 ppm to 70 ppm. In some embodiments, the compound of Formula I or agriculturally acceptable salt thereof is applied from 70 ppm to 80 ppm. In some embodiments, the compound of Formula I or agriculturally acceptable salt thereof is applied from 80 ppm to 90 ppm. In some embodiments, the compound of Formula I or agriculturally acceptable salt thereof is applied from 90 ppm to 100 ppm.

The compound of Formula I and agriculturally acceptable salts thereof may be applied in the form of a composition, including any of the compositions described herein. The compound of Formula I or agriculturally acceptable salt thereof may also be applied in the form of any of the generally used formulation types, for example, as suspension concentrates, emulsifiable concentrates, solutions, powders, dusts, or flowable concentrates. Compositions, including formulations, comprising the compound of Formula I or an agriculturally acceptable salt thereof are described herein below.

The present invention also provides a method for treating a plant or a plant material against pest infestation comprising applying an effective amount of at least one compound of Formula I as described herein or an agricultural acceptable salt thereof to the plant, the plant material, or the vicinity of the plant or plant material so as to thereby treat the plant or plant material against pest infestation.

The present invention also provides a method for controlling and/or preventing of fungal attack on soil, plant, root, foliage, seed, locus of the fungus, or a locus where fungal infestation is to be prevented, wherein the method comprises applying a fungicidally effective amount of at least one compound of Formula I or an agriculturally acceptable salt thereof to the soil, plant, roots, foliage, seed, locus of the fungus, or locus in which the infestation is to be prevented so as to thereby control and/or prevent fungal attack.

The present invention also provides use of at least one compound of Formula I as described herein or an agriculturally acceptable salt thereof as a pesticide.

The present invention also provides use of at least one compound of Formula I as described herein or an agriculturally acceptable salt thereof for treating a locus against pest infestation.

The present invention also provides use of at least one compound of Formula I as described herein or an agriculturally acceptable salt thereof for treating a plant or a plant material against pest infestation.

The present invention also provides use of at least one compound of Formula I as described herein or an agriculturally acceptable salt thereof for controlling and/or preventing of fungal attack on soil, plant, root, foliage, seed, locus of the fungus, or a locus where fungal infestation is to be prevented.

The present invention also provides a compound of Formula I as described herein or an agriculturally acceptable salt thereof for use in treating a locus against pest infestation.

The present invention also provides a compound of Formula I as described herein or an agriculturally acceptable salt thereof for use in treating a plant or a plant material against pest infestation.

The present invention also provides a compound of Formula I as described herein or an agriculturally acceptable salt thereof for use in controlling and/or preventing of fungal attack on soil, plant, root, foliage, seed, locus of the fungus, or a locus where fungal infestation is to be prevented.

In some embodiments, the method or use of the invention is effective for preventing, ameliorating, inhibiting, eliminating and/or delaying the onset of pest infection in farm and industrial produce thereby extending the shelf-life or storage time of said produce.

In some embodiments, the method or use of the invention is effective for improving plant health. In some embodiments, the method or use of the invention is effective for improving the yield of a plant. In some embodiments, the method or use of the invention is effective for increasing biomass of the plant. In some embodiments, the method or use of the invention is effective for increased content of valuable ingredients in the plant. In some embodiments, the method or use of the invention is effective for improving vigor of the plant. In some embodiments, the method or use of the invention is effective for improving plant growth. In some embodiments, the method or use of the invention is effective for improving greenness of leaves. In some embodiments, the method or use of the invention is effective for improving quality of the plant. In some embodiments, the method or use of the invention is effective for improving tolerance of the plant to abiotic and/or biotic stress.

The present invention also provides a composition comprising (i) at least one compound of Formula I as described herein or an agriculturally acceptable salt thereof, and (ii) an agriculturally acceptable carrier, wherein the concentration of the compound of Formula I in the composition is 0.03-1000 g/L.

In some embodiments, the compound of Formula I is in acid form.

In some embodiments, the concentration of the compound of Formula I in the composition is 0.05-1000 g/L. In some embodiments, the concentration of the compound of Formula I in the composition is 0.1-1000 g/L. In some embodiments, the concentration of the compound of Formula I in the composition is 0.5-1000 g/L. In some embodiments, the concentration of the compound of Formula I in the composition is 1-1000 g/L. In some embodiments, the concentration of the compound of Formula I in the composition is 5-1000 g/L. In some embodiments, the concentration of the compound of Formula I in the composition is 10-1000 g/L. In some embodiments, the concentration of the compound of Formula I in the composition is 25-1000 g/L. In some embodiments, the concentration of the compound of Formula I in the composition is 50-1000 g/L. In some embodiments, the concentration of the compound of Formula I in the composition is 100-1000 g/L. In some embodiments, the concentration of the compound of Formula I in the composition is 200-1000 g/L. In some embodiments, the concentration of the compound of Formula I in the composition is 300-1000 g/L. In some embodiments, the concentration of the compound of Formula I in the composition is 400-1000 g/L. In some embodiments, the concentration of the compound of Formula I in the composition is 500-1000 g/L. In some embodiments, the concentration of the compound of Formula I in the composition is 600-1000 g/L. In some embodiments, the concentration of the compound of Formula I in the composition is 700-1000 g/L. In some embodiments, the concentration of the compound of Formula I in the composition is 800-1000 g/L. In some embodiments, the concentration of the compound of Formula I in the composition is 900-1000 g/L.

In some embodiments, the concentration of the compound of Formula I in the composition is greater than 0.03 g/L, 0.05 mg/L, 0.1 g/L, 0.5 g/L, 1 g/L, 5 g/L, 10 g/L, 25 g/l, 50 g/L, 100 g/L, 200 g/L, 300 g/L, 400 g/L, 500 g/L, 600 g/L, 700 g/L, 800 g/L, or 900 g/L.

In some embodiments, the concentration of the compound of Formula I in the composition is 1000 g/L.

In some embodiments, the concentration of the compound of Formula I in the composition is less than 1000 g/L, 900 g/L, 800 g/L, 700 g/L, 600 g/L, 500 g/L, 400 g/L, 300 g/L, 200 g/L, 100 g/L, 50 g/L, 25 g/L, 10 g/L, 5 g/L, 1 g/L, or 0.5 g/L.

Each of the above ranges of concentration may be combined with each other range of concentration. For example, the concentration of “greater than 0.5 g/L” may be combined with the concentration of “less than 900 g/L” to arrive at an embodiment wherein the concentration of the compound of Formula I in the composition is greater than 0.5 g/L and less than 900 g/L.

In some embodiments, the composition is substantially pure of the compound of Formula I or agriculturally acceptable salt thereof.

In some embodiments, the composition comprises at least 80% by weight of the compound of Formula I or agriculturally acceptable salt thereof. In some embodiments, the composition comprises 80-100% by weight of the compound of Formula I or agriculturally acceptable salt thereof. In some embodiments, the composition comprises 85-100% by weight of the compound of Formula I or agriculturally acceptable salt thereof. In some embodiments, the composition comprises 90-100% by weight of the compound of Formula I or agriculturally acceptable salt thereof. In some embodiments, the composition comprises 95-100% by weight of the compound of Formula I or agriculturally acceptable salt thereof. In some embodiments, the composition comprises 80-85% by weight of the compound of Formula I or agriculturally acceptable salt thereof. In some embodiments, the composition comprises 85-90% by weight of the compound of Formula I or agriculturally acceptable salt thereof. In some embodiments, the composition comprises 90-95% by weight of the compound of Formula I or agriculturally acceptable salt thereof.

In some embodiments, the composition comprises a higher concentration of the compound of Formula I than a derivative from Pseudozyma aphidis.

In some embodiments, the composition comprises a higher concentration of the compound of Formula I than any fraction prepared using the procedure of Example 15 of U.S. Pat. Nos. 8,975,213 and 9,161,545.

In some embodiments, the composition comprises a higher concentration of the compound of Formula I than a composition comprising a derivative from Pseudozyma aphidis.

In some embodiments, the composition comprises a higher concentration of the compound of Formula I than the composition comprising spores of Pseudozyma aphidis applied in the Examples of U.S. Pat. Nos. 8,975,213 and 9,161,545, and in particular Examples 6-10 and 16.

In some embodiments, the composition comprises at least one additional pesticide.

In some embodiments, the composition is a formulation. The formulations described herein can be prepared according to procedures that are conventional in the agricultural chemical art.

In some embodiments, the agriculturally acceptable carrier is a solid carrier. In some embodiments, the agriculturally acceptable carrier is a dusty agricultural carrier. In some embodiments, the agriculturally acceptable carrier is a liquid carrier.

In some embodiments, the composition comprises a solid diluent. In some embodiments, the composition comprises a liquid diluent.

In some embodiments, the composition is a concentrated formulation. Concentrated formulations may be dispersed in water, or other liquids, for application.

In some embodiments, the composition is a dust-like or granular formulation. Dust-like or granular formulations may be applied without further treatment.

In some embodiments, the composition is a suspension concentrate. In some embodiments, the composition is an emulsifiable concentrate. In some embodiments, the composition is a solution. In some embodiments, the composition is a powder. In some embodiments, the composition is a dust. In some embodiment, the composition is a flowable concentrate.

The present composition may be employed or prepared in any conventional form, for example, as wettable powders (WP), emulsion concentrates (EC), microemulsion concentrates (MEC), water-soluble powders (SP), water-soluble concentrates (SL), suspoemulsion (SE), oil dispersions (OD), concentrated emulsions (BW) such as oil-in-water and water-in-oil emulsions, sprayable solutions or emulsions, capsule suspensions (CS), suspension concentrates (SC), suspension concentrates, dusts (DP), oil-miscible solutions (OL), seed-dressing products, granules (GR) in the form of microgranules, spray granules, coated granules and absorption granules, granules for soil application or broadcasting, water-soluble granules (SG), water-dispersible granules (WDG), ULV formulations, microcapsules or waxes. These individual formulation types are known in the art.

Wettable powders, which may be compacted to form water dispersible granules, generally comprise a mixture of one or more of the compounds of Formula I or an agriculturally acceptable salt thereof, an inert carrier and surfactants. The concentration of the compound or an agriculturally acceptable salt thereof in the wettable powder may be from about 10 percent to about 90 percent by weight based on the total weight of the wettable powder. In the preparation of wettable powder formulations, the compounds may be compounded with any suitable solid carriers. Examples of suitable solid carriers potentially useful in the present compositions include but are not limited to mineral earths such as silica gels, silicates, talc, kaolin, sericite, attaclay, limestone, bentonite, lime, chalk, bole, mirabilite, loess, clay, dolomite, zeolite, diatomaceous earth, calcium carbonate, calcium sulfate, magnesium sulfate, magnesium oxide, sodium carbonate and bicarbonate, and sodium sulfate; ground synthetic materials; fertilizers such as ammonium sulfate, ammonium phosphate, ammonium nitrate, ureas, and products of vegetable origin, such as cereal meal, tree bark meal, wood meal, and nutshell meal; cellulose powders; and other solid carriers.

Emulsifiable concentrates of the compounds of Formula I or agriculturally acceptable salt thereof may comprise a concentration, such as from about 10 weight percent to about 75 weight percent of the compound or agriculturally acceptable salt thereof, based on the total weight of the concentrate, in a suitable liquid carrier. The compounds may be dissolved in an inert carrier, which is either a water-miscible solvent or a mixture of water-immiscible organic solvents, and emulsifiers. The concentrates may be diluted with water and oil to form spray mixtures in the form of oil-in-water emulsions.

Examples of suitable liquid carriers potentially useful in the present compositions include but are not limited to water; aromatic hydrocarbons such as alkylbenzenes and alkylnaphthalenes; alcohols such as 2-ethoxyethanol, cyclohexanol, and decanol; ethylene glycol; polypropylene glycol; dipropropylene glycol; N,N-dimethylformamide; dimethylsulfoxide; dimethylacetamide; N-alkylpyrrolidones such as N-methyl-2-pyrrolidone; terpenic solvents, paraffins; various oils such as olive, castor, linseed, tung, sesame, corn, peanut, cotton-seed, soybean, rape-seed, or coconut oil; fatty acid esters; ketones such as cyclohexanone, 2-heptanone, isophorone, and 4-hydroxy-4-methyl-2-pentanone; and the like.

In some embodiments, the composition comprises at least one surfactant.

Examples of suitable surfactants include, but are not limited to, non-ionic, anionic, cationic and amphoteric surfactants, or a blend of two or more surfactants. Examples include but are not limited to alkoxylated fatty alcohols, ethoxylated polysorbate (e.g. tween 20), ethoxylated castor oil, lignin sulfonates, fatty acid sulfonates (e.g. lauryl sulfonate), phosphate esters such as phosphate esters of alcohol alkoxylates, phosphate esters of alkylphenol alkoxylates and phosphate esters of styrylphenol ethoxylates, condensates of sulfonated naphthalene and naphthalene derivatives with formaldehyde, condensates of naphthalene or of naphthalenesulfonic acid with phenol and formaldehyde, alkylarylsulfonates, ethoxylated alkylphenols and aryl phenols, polyalkylene glycols, sorbitol esters, alkali metal, sodium salts of lignosulphonates, tristyrylphenol ethoxylate phosphate esters, aliphatic alcohol ethoxylates, alkylphenol ethoxylates, ethylene oxide/propylene oxide block copolymers, graft copolymers and polyvinyl alcohol-vinyl acetate copolymers. Other surfactants known in the art may be used as desired.

In some embodiments, the composition comprises at least one surface active dispersing agent.

Surface active dispersing agents are typically employed in liquid formulations and in an amount of from 0.1 to 20 percent by weight based on the total weight of the formulation. The formulations can also contain other compatible additives, for example, plant growth regulators and other biologically active compounds used in agriculture.

Other ingredients, such as wetting agents, anti-foaming, adhesives, neutralizers, thickeners, binders, sequestrates, fertilizers, biocides, stabilizers, buffers or anti-freeze agents. may also be added to the present compositions in order to increase the stability, density, and viscosity of the described compositions.

Aqueous suspensions comprise suspensions of one or more water-insoluble compounds of Formula I or agriculturally acceptable salt thereof, dispersed in an aqueous vehicle at a concentration in the range from about 5 to about 75 weight percent, based on the total weight of the aqueous suspension. Suspensions are prepared by finely grinding one or more of the compounds, and vigorously mixing the ground material into a carrier comprised of water and surfactants (similar as above). Other components, such as inorganic salts and synthetic or natural gums, may also be added to increase the density and viscosity of the formulation.

Aqueous emulsions comprise emulsions of one or more water-insoluble pesticidally active ingredients emulsified in an aqueous carrier at a concentration typically in the range from about 5 to about 75 weight percent, based on the total weight of the formulation. If the pesticidally active ingredient is a solid it must be dissolved in a suitable water-immiscible solvent prior to the preparation of the aqueous emulsion. Emulsions are prepared by emulsifying the liquid pesticidally active ingredient or water-immiscible solution thereof into an aqueous medium typically with inclusion of surfactants that assist in the formation and stabilization of the emulsion.

Granular formulations, generally contain from about 0.5 to about 50 weight percent, based on the total weight of the granular formulation. Generally, the active compounds are dispersed in an inert carrier which consists entirely or in large part of coarsely divided inert material such as attapulgite, bentonite, diatomite or clay.

Dusts containing the compounds of Formula I or agriculturally acceptable salt thereof may be prepared by intimately mixing one or more of the compounds in powdered form with a suitable dusty agricultural carrier, such as, for example, kaolin clay, ground volcanic rock, and the like. Dusts may contain from about 1 to about 50 weight percent of the compounds, based on the total weight of the dust.

The formulations may additionally contain adjuvant surfactants to enhance deposition, wetting and penetration of the compounds onto the target crop and organism. These adjuvant surfactants may optionally be employed as a component of the formulation or as a tank mix. The amount of adjuvant surfactant may vary from 0.01 to 25.0 percent by volume, based on a spray-volume of water. Suitable adjuvant surfactants include, but are not limited to ethoxylated nonyl phenols, ethoxylated synthetic or natural alcohols, salts of the esters or sulfosuccinic acids, ethoxylated organosilicones, ethoxylated fatty amines and blends of surfactants with mineral or vegetable oils.

In some embodiments, the amount of the compound(s) of Formula I or agriculturally acceptable salt thereof in the composition is from about 0.1%, 0.5%, 1%, 1.5%, 2%, 2.5%, 3%, 3.5%, 4%, 4.5%, 5% to about 90%, 93%, 95%, 98%, 99% based on the total weight of the composition. In some embodiments, the amount of the compound(s) of Formula I or agriculturally acceptable salt thereof in the composition is from 0.1% to 99% based on the total weight of the composition.

In some embodiments, a formulation which comprises a compound of Formula I or agriculturally acceptable salt thereof may be kept at a pH of less than 7. In some embodiment, a formulation which comprises a compound of Formula I or agriculturally acceptable salt thereof may be kept at a pH of 3-6. In some embodiments, the composition or combination has a pH of less than 7. In some embodiment, the composition or combination comprising the compound of Formula I or agriculturally acceptable salt thereof has a pH of 3-6. In some embodiment, the formulation comprising the compound of Formula I or agriculturally acceptable salt thereof has a pH of 3-6.

In some embodiments, the composition or combination comprises a buffer.

The present composition may include additional crop protection agents, for example insecticides, herbicides, fungicides, bactericides, nematicides, molluscicides, growth regulators, biological agents, fertilizers, or mixtures thereof. However, for the avoidance of doubt it is understood that such additional crop protection agents are unnecessary to achieve the desired control and are not antagonistic to the activity of the present compounds. Accordingly, in such embodiments, the other pesticidal compound is used in conjunction and may provide additional protection for the same or for a different pesticidal use. The compounds of Formula I or agriculturally acceptable salt thereof and the pesticidal compound in the combination can generally be present in a weight ratio of from 1:100 to 100:1.

The present invention also provides use of a compound of Formula I in the manufacturing of a composition for treating a locus against pest infestation.

The present invention also provides use of a compound of Formula I in the manufacturing of a composition for treating a plant or a plant material against pest infestation.

The present invention also provides a combination comprising (i) at least one compound of Formula I or an agriculturally acceptable salt thereof as described herein, and (ii) at least one agriculturally acceptable carrier.

In some embodiments, the compound of Formula I is in acid form.

The present invention also provides a combination comprising (i) at least one compound of Formula I or an agriculturally acceptable salt thereof as described herein, and (ii) at least one additional pesticide.

In some embodiments, the compound of Formula I is in acid form.

The present invention also provides a combination comprising (i) any one of the compositions described herein, and (ii) at least one additional pesticide.

In some embodiments, the combination is a mixture. In some embodiments, the combination is a tank mix.

In some embodiments, the combination has a concentration of the compound of Formula I in the composition is 0.03-1000 g/L.

In some embodiments, the concentration of the compound of Formula I in the combination is 0.05-1000 g/L. In some embodiments, the concentration of the compound of Formula I in the combination is 0.1-1000 g/L. In some embodiments, the concentration of the compound of Formula I in the combination is 0.5-1000 g/L. In some embodiments, the concentration of the compound of Formula I in the combination is 1-1000 g/L. In some embodiments, the concentration of the compound of Formula I in the combination is 5-1000 g/L. In some embodiments, the concentration of the compound of Formula I in the combination is 10-1000 g/L. In some embodiments, the concentration of the compound of Formula I in the combination is 25-1000 g/L. In some embodiments, the concentration of the compound of Formula I in the combination is 50-1000 g/L. In some embodiments, the concentration of the compound of Formula I in the combination is 100-1000 g/L. In some embodiments, the concentration of the compound of Formula I in the combination is 200-1000 g/L. In some embodiments, the concentration of the compound of Formula I in the combination is 300-1000 g/L. In some embodiments, the concentration of the compound of Formula I in the combination is 400-1000 g/L. In some embodiments, the concentration of the compound of Formula I in the combination is 500-1000 g/L. In some embodiments, the concentration of the compound of Formula I in the combination is 600-1000 g/L. In some embodiments, the concentration of the compound of Formula I in the combination is 700-1000 g/L. In some embodiments, the concentration of the compound of Formula I in the combination is 800-1000 g/L. In some embodiments, the concentration of the compound of Formula I in the combination is 900-1000 g/L.

In some embodiments, the concentration of the compound of Formula I in the combination is greater than 0.03 g/L, 0.05 g/L, 0.1 g/L, 0.5 g/L, 1 g/L, 5 g/L, 10 g/L, 25 g/L, 50 g/L, 100 g/L, 200 g/L, 300 g/L, 400 g/L, 500 g/L, 600 g/L, 700 g/L, 800 g/L, or 900 g/L.

In some embodiments, the concentration of the compound of Formula I in the combination is 1000 g/L.

In some embodiments, the concentration of the compound of Formula I in the combination is less than 1000 g/L, 900 g/L, 800 g/L, 700 g/L, 600 g/L, 500 g/L, 400 g/L, 300 g/L, 200 g/L, 100 g/L, 50 g/L, 25 g/L, 10 g/L, 5 g/L, 1 g/L, or 0.5 g/L.

Each of the above ranges of concentration may be combined with each other range of concentration. For example, the concentration of “greater than 0.5 g/L” may be combined with the concentration of “less than 900 g/L” to arrive at an embodiment wherein the concentration of the compound of Formula I in the combination is greater than 0.5 g/L and less than 900 g/L.

In some embodiments, the combination is substantially pure of the compound of Formula I or agriculturally acceptable salt thereof.

In some embodiments, the combination comprises at least 80% by weight of the compound of Formula I or agriculturally acceptable salt thereof. In some embodiments, the combination comprises 80-100% by weight of the compound of Formula I or agriculturally acceptable salt thereof. In some embodiments, the combination comprises 85-100% by weight of the compound of Formula I or agriculturally acceptable salt thereof. In some embodiments, the combination comprises 90-100% by weight of the compound of Formula I or agriculturally acceptable salt thereof. In some embodiments, the combination comprises 95-100% by weight of the compound of Formula I or agriculturally acceptable salt thereof. In some embodiments, the combination comprises 80-85% by weight of the compound of Formula I or agriculturally acceptable salt thereof. In some embodiments, the combination comprises 85-90% by weight of the compound of Formula I or agriculturally acceptable salt thereof. In some embodiments, the combination comprises 90-95% by weight of the compound of Formula I or agriculturally acceptable salt thereof.

In some embodiments, the pesticide selected from the group consisting of fungicides, insecticides, nematocides, miticides, arthropodicides, bactericides, acaricides, parasiticides, herbicides and other control agents. Pesticides that may be combined with the compounds described herein (i.e. compounds of Formula I and agriculturally acceptable salts thereof) and the compositions described herein are described below.

The compounds and compositions of the present disclosure may also be combined with fungicides to form fungicidal mixtures and synergistic mixtures thereof. The compounds and compositions of the present disclosure may be applied together with one or more other fungicides to control a wider variety of undesirable diseases. When used in conjunction with other fungicide(s), the presently claimed compounds and compositions may be formulated with the other fungicide(s), tank mixed with the other fungicide(s) or applied sequentially with the other fungicide(s). Such other fungicides may include 2-(thiocyanatomethylthio)-benzothiazole, 2-phenylphenol, 8-hydroxyquinoline sulfate, aminopyrifen ametoctradin, amisulbrom, antimycin, Ampelomyces quisqualis, azaconazole, azoxystrobin, Bacillus subtilis, Bacillus subtilis strain QST713, benalaxyl, benomyl, benthiavalicarb-isopropyl, benzylaminobenzene-sulfonate (BABS) salt, benzovindiflupyr, bicarbonates, biphenyl, bismerthiazol, bitertanol, bixafen, blasticidin-S, borax, Bordeaux mixture, boscalid, bromuconazole, bupirimate, calcium polysulfide, captafol, captan, carbendazim, carboxin, carpropamid, carvone, chlazafenone, chloroneb, chlorothalonil, chlozolinate, Coniothyrium minitans, copper hydroxide, copper, coumoxystrobin, octanoate, copper oxychloride, copper sulfate, copper sulfate (tribasic), cuprous oxide, cyazofamid, cyflufenamid, cymoxanil, cyproconazole, cyprodinil, dazomet, debacarb, diammonium ethylenebis-(dithiocarbamate), dichlofluanid, dichlobentiazox, dipymetitrone, dichlorophen, diclocymet, diclomezine, dichloran, diethofencarb, difenoconazole, difenzoquat ion, diflumetorim, dimethomorph, dimoxystrobin, diniconazole, diniconazole-M, dinobuton, dinocap, diphenylamine, dithianon, dodemorph, dodemorph acetate, dodine, dodine free base, edifenphos, enestrobin, epoxiconazole, ethaboxam, enoxastrobin, ethoxyquin, etridiazole, famoxadone, fenamidone, fenarimol, fenbuconazole, fenfuram, fenhexamid, fenoxanil, fenpiclonil, fenpropidin, fenpropimorph, fenpyrazamine, fentin, fentin acetate, fentin hydroxide, ferbam, ferimzone, fluazinam, fenaminstrobin, fluoxapiprolin, flufenoxystrobin, fludioxonil, flumetylsulforim, flufenoxadiazam, florylpicoxamid, fluopimomide, flufenoxadiazam, fenpicoxamid, fluindapyr, flumorph, fluopicolide, flubenetram, fluopyram, fluoroimide, fluoxastrobin, fluquinconazole, flusilazole, flusulfamide, flutianil, flutolanil, flutriafol, fluxapyroxad, folpet, formaldehyde, fosetyl, fosetyl-aluminium, fuberidazole, furalaxyl, furametpyr, guazatine, guazatine acetates, GY-81, hexachlorobenzene, hexaconazole, hymexazol, imazalil, imazalil sulfate, imibenconazole, iminoctadine, iminoctadine triacetate, iminoctadine tris(albesilate), inpyrfiluxarn, iodocarb, ipconazole, ipfentrifluconazole, ipfenpyrazolone, iprobenfos, iprodione, iprovalicarb, isofetamid, isoflucypram, isoprothiolane, isopyrazam, isotianil, kasugamycin, kasugamycin hydrochloride hydrate, kresoxim-methyl, laminarin, mancopper, mancozeb, mandestrobin, mandipropamid, maneb, mefentrifluconazole, metyltetraprole, metarylpicoxamid, mepanipyrim, mepronil, meptyl-dinocap, mercuric chloride, mercuric oxide, mercurous chloride, metalaxyl, mefenoxam, metalaxyl-M, metam, metam-ammonium, metam-potassium, metam-sodium, metconazole, methasulfocarb, methyl iodide, methyl isothiocyanate, metiram, metominostrobin, metrafenone, mildiomycin, myclobutanil, nabam, natamycin, nitrothal-isopropyl, nuarimol, octhilinone, ofurace, oleic acid (fatty acids), orysastrobin, oxadixyl, oxathiapiprolin, oxine-copper, oxpoconazole fumarate, oxycarboxin, pefurazoate, penconazole, pencycuron, penflufen, pentachlorophenol, pentachlorophenyl laurate, penthiopyrad, phenylmercury acetate, phosphonic acid, phthalide, picarbutrazox, picoxystrobin, pyraziflumid, polyoxin B, polyoxins, pyrisoxazole, pyriminostrobin, polyoxorim, potassium bicarbonate, potassium hydroxyquinoline sulfate, probenazole, prochloraz, procymidone, propamocarb, propamocarb hydrochloride, propiconazole, propineb, proquinazid, prothioconazole, pydiflumetofen, pyridachiometyl, pyraclostrobin, pyrametostrobin, pyraoxystrobin, pyrapropoyne, pyrazophos, pyribencarb, pyributicarb, pyrifenox, pyrimethanil, pyriofenone, pyroquilon, quinoclamine, quinoxyfen, quinofumelin, quintozene, Reynoutria sachalinensis extract, sedaxane, silthiofam, simeconazole, sodium 2-phenylphenoxide, sodium bicarbonate, sodium pentachlorophenoxide, spiroxamine, sulfur, SYP-Z071, SYP-Z048, tar oils, tebuconazole, tebufloquin, tecnazene, tetraconazole, thiabendazole, thifluzamide, thiophanate-methyl, thiram, tiadinil, tolclofos-methyl, tolylfluanid, triadimefon, triadimenol, triazoxide, triclopyricarb, tricyclazole, tridemorph, trifloxystrobin, triflumizole, triforine, triticonazole, validamycin, valifenalate, valiphenal, vinclozolin, zineb, ziram, zoxamide, Candida oleophila, Fusarium oxvsporum, Gliocladium spp., Phlebiopsis gigantea. Streptomyces griseoviridis, Trichoderma spp., (RS)—N-(3,5-dichlorophenyl)-2-(methoxymethyl)-succinimide, 1,2-dichloropropane, 1,3-dichloro-1,1,3,3-tetrafluoroacetone hydrate, 1-chloro-2,4-dinitronaphthalene, 1-chloro-2-nitropropane, 2-(2-heptadecyl-2-imidazolin-1-yl)ethanol, 2,3-dihydro-5-phenyl-1,4-dithi-ine 1,1,4,4-tetraoxide, 2-methoxyethylmercury acetate, 2-methoxyethylmercury chloride, 2-methoxyethylmercury silicate, 3-(4-chlorophenyl)-5-methylrhodanine, 4-(2-nitroprop-1-enyl)phenyl thiocyanateme, ampropylfos, anilazine, azithiram, barium polysulfide, Bayer 32394, benodanil, benquinox, bentaluron, benzamacril; benzamacril-isobutyl, benzamorf, binapacryl, bis(methylmercury) sulfate, bis(tributyltin) oxide, buthiobate, cadmium calcium copper zinc chromate sulfate, carbamorph, CECA, chlobenthiazone, chloraniformethan, chlorfenazole, chlorquinox, climbazole, cyclafuramid, cypendazole, cyprofuram, decafentin, dichlone, dichlozoline, diclobutrazol, dimethirimol, dinocton, dinosulfon, dinoterbon, dipyrithione, ditalimfos, dodicin, drazoxolon, EBP, ESBP, etaconazole, etem, ethirim, fenaminosulf, fenapanil, fenitropan, 5-fluorocytosine and profungicides thereof, fluotrimazole, furcarbanil, furconazole, furconazole-cis, furmecyclox, furophanate, glyodine, griseofulvin, halacrinate, Hercules 3944, hexylthiofos, ICIA0858, isopamphos, isovaledione, mebenil, mecarbinzid, metazoxolon, methfuroxam, methylmercury dicyandiamide, metsulfovax, milneb, mucochloric anhydride, myclozolin, N-3,5-dichlorophenyl-succinimide, N-3-nitrophenylitaconimide, natamycin, N-ethylmercurio-4-toluenesulfonanilide, nickel bis(dimethyldithiocarbamate), OCH, phenylmercury dimethyldithiocarbamate, phenylmercury nitrate, phosdiphen, picolinamide UK-2A and derivatives thereof, prothiocarb; prothiocarb hydrochloride, pyracarbolid, pyridinitril, pyroxychlor, pyroxyfur, quinacetol; quinacetol sulfate, quinazamid, quinconazole, rabenzazole, salicylanilide, SSF-109, sultropen, tecoram, thiadifluor, thicyofen, thiochlorfenphim, thiophanate, thioquinox, tioxymid, tolprocarb, triamiphos, triarimol, triazbutil, trichlamide, urbacid, zarilamide, and any combinations thereof.

Additionally, the compounds and compositions of the present invention may be combined with other pesticides, including insecticides, nematocides, miticides, arthropodicides, bactericides or combinations thereof that are compatible with the compounds and compositions of the present to form pesticidal mixtures and synergistic mixtures thereof. The fungicidal compounds and compositions of the present disclosure may be applied in conjunction with one or more other pesticides to control a wider variety of undesirable pests. When used in conjunction with other pesticides, the presently claimed compounds and compositions may be formulated with the other pesticide(s), tank mixed with the other pesticide(s) or applied sequentially with the other pesticide(s). Typical insecticides include, but are not limited to: antibiotic insecticides such as allosamidin and thuringiensin; macrocyclic lactone insecticides such as spinosad and spinetoram; avermectin insecticides such as abamectin, doramectin, emamectin, eprinomectin, ivermectin and selamectin; milbemycin insecticides such as lepimectin, milbemectin, milbemycin oxime and moxidectin; arsenical insecticides such as calcium arsenate, copper acetoarsenite, copper arsenate, lead arsenate, potassium arsenite and sodium arsenite; botanical insecticides such as anabasine, azadirachtin, d-limonene, nicotine, pyrethrins, cinerins, cinerin I, cinerin II, jasmolin I, jasmolin II, pyrethrin I, pyrethrin II, quassia, rotenone, ryania and sabadilla; carbamate insecticides such as bendiocarb and carbaryl; benzofuranyl methylcarbamate insecticides such as benfuracarb, carbofuran, carbosulfan, decarbofuran and furathiocarb; dimethylcarbamate insecticides dimitan, dimetilan, hyquincarb and pirimicarb; oxime carbamate insecticides such as alanycarb, aldicarb, aldoxycarb, butocarboxim, butoxycarboxim, methomyl, nitrilacarb, oxamyl, tazimcarb, thiocarboxime, thiodicarb and thiofanox; phenyl methylcarbamate insecticides such as allyxycarb, aminocarb, bufencarb, butacarb, carbanolate, cloethocarb, dicresyl, dioxacarb, EMPC, ethiofencarb, fenethacarb, fenobucarb, isoprocarb, methiocarb, metolcarb, mexacarbate, promacyl, promecarb, propoxur, trimethacarb, XMC and xylylcarb; dessicant insecticides such as boric acid, diatomaceous earth and silica gel; diamide insecticides such as chlorantraniliprole, cyantraniliprole and flubendiamide; dinitrophenol insecticides such as dinex, dinoprop, dinosam and DNOC; fluorine insecticides such as barium hexafluorosilicate, cryolite, sodium fluoride, sodium hexafluorosilicate and sulfluramid; formamidine insecticides such as amitraz, chlordimeform, formetanate and formparanate; fumigant insecticides such as acrylonitrile, carbon disulfide, carbon tetrachloride, chloroform, chloropicrin, para-dichlorobenzene, 1,2-dichloropropane, ethyl formate, ethylene dibromide, ethylene dichloride, ethylene oxide, hydrogen cyanide, iodomethane, methyl bromide, methylchloroform, methylene chloride, naphthalene, phosphine, sulfuryl fluoride and tetrachloroethane; inorganic insecticides such as borax, calcium polysulfide, copper oleate, mercurous chloride, potassium thiocyanate and sodium thiocyanate; chitin synthesis inhibitors such as bistrifluoron, buprofezin, chlorfluazuron, cyromazine, diflubenzuron, flucycloxuron, flufenoxuron, hexaflumuron, lufenuron, novaluron, noviflumuron, penfluoron, teflubenzuron and triflumuron; juvenile hormone mimics such as epofenonane, fenoxycarb, hydroprene, kinoprene, methoprene, pyriproxyfen and triprene; juvenile hormones such as juvenile hormone I, juvenile hormone II and juvenile hormone III; moulting hormone agonists such as chromafenozide, halofenozide, methoxyfenozide and tebufenozide; moulting hormones such as a-ecdysone and ecdysterone; moulting inhibitors such as diofenolan; precocenes such as precocene I, precocene II and precocene III; unclassified insect growth regulators such as dicyclanil; nereistoxin analogue insecticides such as bensultap, cartap, thiocyclam and thiosultap; nicotinoid insecticides such as flonicamid; nitroguanidine insecticides such as clothianidin, dinotefuran, imidacloprid and thiamethoxam; nitromethylene insecticides such as nitenpyram and nithiazine; pyridylnethyl-amine insecticides such as acetamiprid, imidacloprid, nitenpyram and thiacloprid; organochlorine insecticides such as bromo-DDT, camphechlor, DDT, pp′-DDT, ethyl-DDD, HCH, gamma-HCH, lindane, methoxychlor, pentachlorophenol and TDE; cyclodiene insecticides such as aldrin, bromocyclen, chlorbicyclen, chlordane, chlordecone, dieldrin, dilor, endosulfan, alpha-endosulfan, endrin, HEOD, heptachlor, HHDN, isobenzan, isodrin, kelevan and mirex; organophosphate insecticides such as bromfenvinfos, chlorfenvinphos, crotoxyphos, dichlorvos, dicrotophos, dimethylvinphos, fospirate, heptenophos, methoerotophos, mevinphos, monocrotophos, naled, naftalofos, phosphamidon, propaphos, TEPP and tetrachlorvinphos; organothiophosphate insecticides such as dioxabenzofos, fosmethilan and phenthoate; aliphatic organothiophosphate insecticides such as acethion, amiton, cadusafos, chlorethoxyfos, chlormephos, demephion, demephion-O, demephion-S, demeton, demeton-O, demeton-S, demeton-methyl, demeton-O-methyl, demeton-S-methyl, demeton-S-methylsulphon, disulfoton, ethion, ethoprophos, IPSP, isothioate, malathion, methacrifos, oxydemeton-methyl, oxydeprofos, oxydisulfoton, phorate, sulfotep, terbufos and thiometon; aliphatic amide organothiophosphate insecticides such as amidithion, cyanthoate, dimethoate, ethoate-methyl, formothion, mecarbam, omethoate, prothoate, sophamide and vamidothion; oxime organothiophosphate insecticides such as chlorphoxim, phoxim and phoxim-methyl; heterocyclic organothiophosphate insecticides such as azamethiphos, coumaphos, coumithoate, dioxathion, endothion, menazon, morphothion, phosalone, pyraclofos, pyridaphenthion and quinothion; benzothiopyran organothiophosphate insecticides such as dithicrofos and thicrofos; benzotriazine organothiophosphate insecticides such as azinphos-ethyl and azinphos-methyl; isoindole organothiophosphate insecticides such as dialifos and phosmet; isoxazole organothiophosphate insecticides such as isoxathion and zolaprofos; pyrazolopyrimidine organothiophosphate insecticides such as chlorprazophos and pyrazophos; pyridine organothiophosphate insecticides such as chlorpyrifos and chlorpyrifos-methyl; pyrimidine organothiophosphate insecticides such as butathiofos, diazinon, etrimfos, lirimfos, pirimiphos-ethyl, pirimiphos-methyl, primidophos, pyrimitate and tebupirimfos; quinoxaline organothiophosphate insecticides such as quinalphos and quinalphos-methyl; thiadiazole organothiophosphate insecticides such as athidathion, lythidathion, methidathion and prothidathion; triazole organothiophosphate insecticides such as isazofos and triazophos; phenyl organothiophosphate insecticides such as azothoate, bromophos, bromophos-ethyl, carbophenothion, chlorthiophos, cyanophos, cythioate, dicapthon, dichlofenthion, etaphos, famphur, fenchlorphos, fenitrothion fensulfothion, fenthion, fenthion-ethyl, heterophos, jodfenphos, mesulfenfos, parathion, parathion-methyl, phenkapton, phosnichlor, profenofos, prothiofos, sulprofos, temephos, trichlormetaphos-3 and trifenofos; phosphonate insecticides such as butonate and trichlorfon; phosphonothioate insecticides such as mecarphon; phenyl ethylphosphonothioate insecticides such as fonofos and trichloronat; phenyl phenylphosphonothioate insecticides such as cyanofenphos, EPN and leptophos; phosphoramidate insecticides such as crufomate, fenamiphos, fosthietan, mephosfolan, phosfolan and pirimetaphos; phosphoramidothioate insecticides such as acephate, isocarbophos, isofenphos, isofenphos-methyl, methamidophos and propetamphos; phosphorodiamide insecticides such as dimefox, mazidox, mipafox and schradan; oxadiazine insecticides such as indoxacarb; oxadiazoline insecticides such as metoxadiazone; phthalimide insecticides such as dialifos, phosmet and tetramethrin; pyrazole insecticides such as tebufenpyrad, tolefenpyrad; phenylpyrazole insecticides such as acetoprole, ethiprole, fipronil, pyrafluprole, pyriprole and vaniliprole; pyrethroid ester insecticides such as acrinathrin, allethrin, bioallethrin, barthrin, bifenthrin, bioethanomethrin, cyclethrin, cycloprothrin, cyfluthrin, beta-cyfluthrin, cyhalothrin, gamma-cyhalothrin, lambda-cyhalothrin, cypermethrin, alpha-cypermethrin, beta-cypermethrin, theta-cypermethrin, zeta-cypermethrin, cyphenothrin, deltamethrin, dimefluthrin, dimethrin, empenthrin, fenfluthrin, fenpirithrin, fenpropathrin, fenvalerate, esfenvalerate, flucythrinate, fluvalinate, tau-fluvalinate, furethrin, imiprothrin, meperfluthrin, metofluthrin, permethrin, biopermethrin, transpermethrin, phenothrin, prallethrin, profluthrin, pyresmethrin, resmethrin, bioresmethrin, cismethrin, tefluthrin, terallethrin, tetramethrin, tetramethylfluthrin, tralomethrin and transfluthrin; pyrethroid ether insecticides such as etofenprox, flufenprox, halfenprox, protrifenbute and silafluofen; pyrimidinamine insecticides such as flufenerim and pyrimidifen; pyrrole insecticides such as chlorfenapyr; tetramic acid insecticides such as spirotetramat; tetronic acid insecticides such as spiromesifen; thiourea insecticides such as diafenthiuron; urea insecticides such as flucofuron and sulcofuron; and unclassified insecticides such as closantel, copper naphthenate, crotamiton, EXD, fenazaflor, fenoxacrim, hydramethylnon, isoprothiolane, malonoben, metaflumizone, nifluridide, plifenate, pyridaben, pyridalyl, pyrifluquinazon, rafoxanide, sulfoxaflor, triarathene and triazamate, and any combinations thereof.

The compounds and compositions of the present invention may be combined with herbicides that are compatible with the compounds and compositions of the present invention to form pesticidal mixtures and synergistic mixtures thereof. The fungicidal compounds and compositions of the present disclosure may be applied in conjunction with one or more herbicides to control a wide variety of undesirable plants. When used in conjunction with herbicides, the presently claimed compounds and compositions may be formulated with the herbicide(s), tank mixed with the herbicide(s) or applied sequentially with the herbicide(s). Typical herbicides include, but are not limited to: amide herbicides such as allidochlor, beflubutamid, benzadox, benzipram, bromobutide, cafenstrole, CDEA, cyprazole, dimethenamid, dimethenamid-P, diphenamid, epronaz, etnipromid, fentrazamide, flupoxam, fomesafen, halosafen, isocarbamid, isoxaben, napropamide, naptalam, pethoxamid, propyzamide, quinonamid and tebutam; anilide herbicides such as chloranocryl, cisanilide, clomeprop, cypromid, diflufenican, etobenzanid, fenasulam, flufenacet, flufenican, mefenacet, mefluidide, metamifop, monalide, naproanilide, pentanochlor, picolinafen and propanil; arylalanine herbicides such as benzoylprop, flamprop and flamprop-M; chloroacetanilide herbicides such as acetochlor, alachlor, butachlor, butenachlor, delachlor, diethatyl, dimethachlor, metazachlor, metolachlor, S-metolachlor, pretilachlor, propachlor, propisochlor, prynachlor, terbuchlor, thenylchlor and xylachlor; sulfonanilide herbicides such as benzofluor, perfluidone, pyrimisulfan and profluazol; sulfonamide herbicides such as asulam, carbasulam, fenasulam and oryzalin; thioamide herbicides such as chlorthiamid; antibiotic herbicides such as bilanafos; benzoic acid herbicides such as chloramben, dicamba, 2,3,6-TBA and tricamba; pyrimidinyloxybenzoic acid herbicides such as bispyribac and pyriminobac; pyrimidinylthiobenzoic acid herbicides such as pyrithiobac; phthalic acid herbicides such as chlorthal; picolinic acid herbicides such as aminopyralid, clopyralid and picloram; quinolinecarboxylic acid herbicides such as quinclorac and quinmerac; arsenical herbicides such as cacodylic acid, CMA, DSMA, hexaflurate, MAA, MAMA, MSMA, potassium arsenite and sodium arsenite; benzoylcyclohexanedione herbicides such as mesotrione, sulcotrione, tefuryltrione and tembotrione; benzofuranyl alkylsulfonate herbicides such as benfuresate and ethofumesate; benzothiazole herbicides such as benzazolin; carbamate herbicides such as asulam, carboxazole chlorprocarb, dichlormate, fenasulam, karbutilate and terbucarb; carbanilate herbicides such as barban, BCPC, carbasulam, carbetamide, CEPC, chlorbufam, chlorpropham, CPPC, desmedipham, phenisopham, phenmedipham, phenmedipham-ethyl, propham and swep; cyclohexene oxime herbicides such as alloxydim, butroxydim, clethodim, cloproxydim, cycloxydim, profoxydim, sethoxydim, tepraloxydim and tralkoxydim; cyclopropylisoxazole herbicides such as isoxachlortole and isoxaflutole; dicarboximide herbicides such as cinidon-ethyl, flumezin, flumiclorac, flumioxazin and flumipropyn; dinitroaniline herbicides such as benfluralin, butralin, dinitramine, ethalfluralin, fluchloralin, isopropalin, methalpropalin, nitralin, oryzalin, pendimethalin, prodiamine, profluralin and trifluralin; dinitrophenol herbicides such as dinofenate, dinoprop, dinosam, dinoseb, dinoterb, DNOC, etinofen and medinoterb; diphenyl ether herbicides such as ethoxyfen; nitrophenyl ether herbicides such as acifluorfen, aclonifen, bifenox, chlomethoxyfen, chlornitrofen, etnipromid, fluorodifen, fluoroglycofen, fluoronitrofen, fomesafen, furyloxyfen, halosafen, lactofen, nitrofen, nitrofluorfen and oxyfluorfen; dithiocarbamate herbicides such as dazomet and metam; halogenated aliphatic herbicides such as alorac, chloropon, dalapon, flupropanate, hexachloroacetone, iodomethane, methyl bromide, monochloroacetic acid, SMA and TCA; imidazolinone herbicides such as imazamethabenz, imazamox, imazapic, imazapyr, imazaquin and imazethapyr; inorganic herbicides such as ammonium sulfamate, borax, calcium chlorate, copper sulfate, ferrous sulfate, potassium azide, potassium cyanate, sodium azide, sodium chlorate and sulfuric acid; nitrile herbicides such as bromobonil, bromoxynil, chloroxynil, dichlobenil, iodobonil, ioxynil and pyraclonil; organophosphorus herbicides such as amiprofos-methyl, anilofos, bensulide, bilanafos, butamifos, 2,4-DEP, DMPA, EBEP, fosamine, glufosinate, glufosinate-P, glyphosate and piperophos; phenoxy herbicides such as bromofenoxim, clomeprop, 2,4-DEB, 2,4-DEP, difenopenten, disul, erbon, etnipromid, fenteracol and trifopsime; oxadiazoline herbicides such as methazole, oxadiargyl, oxadiazon; oxazole herbicides such as fenoxasulfone; phenoxyacetic herbicides such as 4-CPA, 2,4-D, 3,4-DA, MCPA, MCPA-thioethyl and 2,4,5-T; phenoxybutyric herbicides such as 4-CPB, 2,4-DB, 3,4-DB, MCPB and 2,4,5-TB; phenoxypropionic herbicides such as cloprop, 4-CPP, dichlorprop, dichlorprop-P, 3,4-DP, fenoprop, mecopropand mecoprop-P; aryloxyphenoxypropionic herbicides such as chlorazifop, clodinafop, clofop, cyhalofop, diclofop, fenoxaprop, fenoxaprop-P, fenthiaprop, fluazifop, fluazifop-P, haloxyfop, haloxyfop-P, isoxapyrifop, metamifop, propaquizafop, quizalofop, quizalofop-P and trifop; phenylenediamine herbicides such as dinitramine and prodiamine; pyrazole herbicides such as pyroxasulfone; benzoylpyrazole herbicides such as benzofenap, pyrasulfotole, pyrazolynate, pyrazoxyfen, and topramezone; phenylpyrazole herbicides such as fluazolate, nipyraclofen, pioxaden and pyraflufen; pyridazine herbicides such as credazine, pyridafol and pyridate; pyridazinone herbicides such as brompyrazon, chloridazon, dimidazon, flufenpyr, metflurazon, norflurazon, oxapyrazon and pydanon; pyridine herbicides such as aminopyralid, cliodinate, clopyralid, dithiopyr, fluoroxypyr, haloxydine, picloram, picolinafen, pyriclor, thiazopyr and triclopyr; pyrimidinediamine herbicides such as iprymidam and tioclorim; quatemary ammonium herbicides such as cyperquat, diethamquat, difenzoquat, diquat, morfamquat and paraquat; thiocarbamate herbicides such as butylate, cycloate, di-allate, EPTC, esprocarb, ethiolate, isopolinate, methiobencarb, molinate, orbencarb, pebulate, prosulfocarb, pyributicarb, sulfallate, thiobencarb, tiocarbazil, tri-allate and vernolate; thiocarbonate herbicides such as dimexano, EXD and proxan; thiourea herbicides such as methiuron; triazine herbicides such as dipropetryn, indaziflam, triaziflam and trihydroxytriazine; chlorotriazine herbicides such as atrazine, chlorazine, cyanazine, cyprazine, eglinazine, ipazine, mesoprazine, procyazine, proglinazine, propazine, sebuthylazine, simazine, terbuthylazine and trietazine; methoxytriazine herbicides such as atraton, methometon, prometon, secbumeton, simeton and terbumeton; methylthiotriazine herbicides such as ametryn, aziprotryne, cyanatryn, desmetryn, dimethametryn, methoprotryne, prometryn, simetryn and terbutryn; triazinone herbicides such as ametridione, amibuzin, hexazinone, isomethiozin, metamitron and metribuzin; triazole herbicides such as amitrole, cafenstrole, epronaz and flupoxam; triazolone herbicides such as amicarbazone, bencarbazone, carfentrazone, flucarbazone, ipfencarbazone, propoxycarbazone, sulfentrazone and thiencarbazone-methyl; triazolopyrimidine herbicides such as cloransulam, diclosulam, florasulam, flumetsulam, metosulam, penoxsulam and pyroxsulam; uracil herbicides such as benzfendizone, bromacil, butafenacil, flupropacil, isocil, lenacil, saflufenacil and terbacil; urea herbicides such as benzthiazuron, cumyluron, cycluron, dichloralurea, diflufenzopyr, isonoruron, isouron, methabenzthiazuron, monisouron and noruron; phenylurea herbicides such as anisuron, buturon, chlorbromuron, chloreturon, chlorotoluron, chloroxuron, daimuron, difenoxuron, dimefuron, diuron, fenuron, fluometuron, fluothiuron, isoproturon, linuron, methiuron, methyldymron, metobenzuron, metobromuron, metoxuron, monolinuron, monuron, neburon, parafluoron, phenobenzuron, siduron, tetrafluoron and thidiazuron; pyrimidinylsulfonylurea herbicides such as amidosulfuron, azimsulfuron, bensulfuron, chlorimuron, cyclosulfamuron, ethoxysulfuron, flazasulfuron, flucetosulfuron, flupyrsulfuron, foramsulfuron, halosulfuron, imazosulfuron, mesosulfuron, metazosulfuron, nicosulfuron, orthosulfamuron, oxasulfuron, primisulfuron, propyrisulfuron, pyrazosulfuron, rimsulfuron, sulfometuron, sulfosulfuron and trifloxysulfuron; triazinylsulfonylurea herbicides such as chlorsulfuron, cinosulfuron, ethametsulfuron, iodosulfuron, metsulfuron, prosulfuron, thifensulfuron, triasulfuron, tribenuron, triflusulfuron and tritosulfuron; thiadiazolylurea herbicides such as buthiuron, ethidimuron, tebuthiuron, thiazafluoron and thidiazuron; and unclassified herbicides such as acrolein, allyl alcohol, aminocyclopyrachlor, azafenidin, bentazone, benzobicyclon, bicyclopyrone, buthidazole, calcium cyanamide, cambendichlor, chlorfenac, chlorfenprop, chlorflurazole, chlorflurenol, cinmethylin, clomazone, CPMF, cresol, cyanamide, ortho-dichlorobenzene, dimepiperate, endothal, fluoromidine, fluridone, fluorochloridone, flurtamone, fluthiacet, indanofan, methyl isothiocyanate, OCH, oxaziclomefone, pentachlorophenol, pentoxazone, phenylmercury acetate, prosulfalin, pyribenzoxim, pyriftalid, quinoclamine, rhodethanil, sulglycapin, thidiazimin, tridiphane, trimeturon, tripropindan and tritac.

The compounds of the present disclosure may also be combined with other fungicides to form fungicidal mixtures and synergistic mixtures thereof. The fungicidal compounds of the present disclosure are often applied in conjunction with one or more other fungicides to control a wider variety of undesirable diseases. When used in conjunction with other fungicide(s), the presently claimed compounds may be formulated with the other fungicide(s), tank-mixed with the other fungicide(s) or applied sequentially with the other fungicide(s). Such other fungicides may include 2-(thiocyanatomethylthio)-benzothiazole, 2-phenylphenol, 8-hydroxyquinoline sulfate, ametoctradin, aminopyrifen, amisulbrom, antimycin, Ampelomyces quisqualis, azaconazole, Bacillus subtilis, Bacillus subtilis strain QST713, benalaxyl, benomyl, benthiavalicarb-isopropyl, benzovindiflupyr, benzylaminobenzene-sulfonate (BABS) salt, bicarbonates, biphenyl, bismerthiazol, bitertanol, bixafen, blasticidin-S, borax, Bordeaux mixture, boscalid, bromuconazole, bupirimate, calcium polysulfide, captafol, captan, carbendazim, carboxin, carpropamid, carvone, chlazafenone, chloroneb, chlorothalonil, chlozolinate, Coniothyrium minitans, copper hydroxide, copper octanoate, copper oxychloride, copper sulfate, copper sulfate (tribasic), cuprous oxide, cyazofamid, cyflufenamid, cymoxanil, cyproconazole, cyprodinil, dazomet, debacarb, diammonium ethylenebis-(dithiocarbamate), dichlofluanid, dichlorophen, diclocymet, diclomezine, dichloran, diethofencarb, difenoconazole, difenzoquat ion, diflumetorim, dimethomorph, dimoxystrobin, diniconazole, diniconazole-M, dinobuton, dinocap, diphenylamine, dithianon, dodemorph, dodemorph acetate, dodine, dodine free base, edifenphos, enestrobin, enestroburin, epoxiconazole, ethaboxam, ethoxyquin, etridiazole, famoxadone, fenamidone, fenarimol, fenbuconazole, fenfuram, fenhexamid, fenoxanil, fenpiclonil, fenpropidin, fenpropimorph, fenpyrazamine, fentin, fentin acetate, fentin hydroxide, ferbam, ferimzone, fluazinam, fludioxonil, Flufenoxadiazam fluindapyr, flumorph, fluopicolide, fluopyram, fluoroimide, fluoxapiprolin, fluoxastrobin, fluquinconazole, flusilazole, flusulfamide, flutianil, flutolanil, flutriafol, fluxapyroxad, folpet, formaldehyde, fosetyl, fosetyl-aluminium, fuberidazole, furalaxyl, furametpyr, guazatine, guazatine acetates, GY-81, hexachlorobenzene, hexaconazole, hymexazol, imazalil, imazalil sulfate, imibenconazole, iminoctadine, iminoctadine triacetate, iminoctadine tris(albesilate), inpyrfluxam, iodocarb, ipconazole, ipfenpyrazolone, iprobenfos, iprodione, iprovalicarb, isofetamide, isoflucypram, isoprothiolane, isopyrazam, isotianil, kasugamycin, kasugamycin hydrochloride hydrate, kresoxium-methyl, laminarin, mancopper, mancozeb, mandipropamid, maneb, mefenoxam, mepanipyrim, mepronil, meptyl-dinocap, mercuric chloride, mercuric oxide, mercurous chloride, metalaxyl, metalaxyl-M, metam, metam-ammonium, metam-potassium, metam-sodium, metconazole, methasulfocarb, methyl iodide, methyl isothiocyanate, metiram, metominostrobin, metrafenone, mildiomycin, myclobutanil, nabam, nitrothal-isopropyl, nuarimol, octhilinone, ofurace, oleic acid (fatty acids), orysastrobin, oxadixyl, oxathiapiprolin, oxine-copper, oxpoconazole fumarate, oxycarboxin, pefurazoate, penconazole, pencycuron, penflufen, pentachlorophenol, pentachlorophenyl laurate, penthiopyrad, phenylmercury acetate, phosphonic acid, phthalide, picoxystrobin, polyoxin B, polyoxins, polyoxorim, potassium bicarbonate, potassium hydroxyquinoline sulfate, probenazole, prochloraz, procymidone, propamocarb, propamocarb hydrochloride, propiconazole, propineb, proquinazid, prothioconazole, pydiflumetofen, pyrametostrobin, pyraoxystrobin, pyraclostrobin, pyraziflumid, pyrazophos, pyribencarb, pyributicarb, pyrifenox, pyrimethanil, pyriofenone, pyroquilon, quinoclamine, quinoxyfen, quintozene, Reynoutria sachalinensis extract, sedaxane, silthiofam, simeconazole, sodium 2-phenylphenoxide, sodium bicarbonate, sodium pentachlorophenoxide, spiroxamine, sulfur, SYP-Z048, tar oils, tebuconazole, tebufloquin, tecnazene, tetraconazole, thiabendazole, thifluzamide, thiophanate-methyl, thiram, tiadinil, tolclofos-methyl, tolylfluanid, triadimefon, triadimenol, triazoxide, tricyclazole, tridemorph, trifloxystrobin, triflumizole, triforine, triticonazole, validamycin, valifenalate, valiphenal, vinclozolin, zineb, ziram, zoxamide, Candida oleophila, Fusarium oxysporum, Gliocladium spp., Phlebiopsis gigantea, Streptomyces griscoviridis, Trichoderma spp., S,)-/V-(3,5-dichlorophenyl)-2-(methoxymethyl)-succinimide, 1,2-dichloropropane, 1,3-dichloro-1,1,3,3-tetrafluoroacetone hydrate, 1-chloro-2,4-dinitronaphthalene, 1-chloro-2-nitropropane, 2-(2-heptadecyl-2-imidazolin-1-yl)ethanol, 2,3-dihydro-5-phenyl-1,4-dithi-ine 1,1,4,4-tetraoxide, 2-methoxyethylmercury acetate, 2-methoxyethylmercury chloride, 2-methoxyethylmercury silicate, 3-(4-chlorophenyl)-5-methylrhodanine, 4-(2-nitroprop-I-enyl)phenyl thiocyanateme, ampropylfos, anilazine, azithiram, barium polysulfide, Bayer 32394, benodanil, benquinox, bentaluron, benzamacril; benzamacril-isobutyl, benzamorf, binapacryl, bis(methylmercury) sulfate, bis(tributyltin) oxide, buthiobate, cadmium calcium copper zinc chromate sulfate, carbamorph, CECA, chlobenthiazone, chloraniformethan, chlorfenazole, chlorquinox, climbazole, copper bis(3-phenylsalicylate), copper zinc chromate, coumoxystrobin, cufraneb, cupric hydrazinium sulfate, cuprobam, cyclafuramid, cypendazole, cyprofuram, decafentin, dichlobentiazox, dichlone, dichlozoline, diclobutrazol, dimethirimol, dinocton, dinosulfon, dinoterbon, dipymetitrone, dipyrithione, ditalimfos, dodicin, drazoxolon, EBP, enoxastrobin, ESBP, etaconazole, etem, ethirim, fenaminstrobin, fenaminosulf, fenapanil, fenitropan, fenpicoxamid, florylpicoxamid, flufenoxystrobin, fluopimomide, fluotrimazole, furcarbanil, furconazole, furconazole-cis, furmecyclox, furophanate, glyodine, griseofulvin, halacrinate, Hercules 3944, hexylthiofos, ICIA0858, ipfentrifluconazole, ipflufenoquin, isopamphos, isovaledione, mandestrobin, mebenil, mecarbinzid, mefentrifluconazole, metazoxolon, methfuroxam, methylmercury dicyandiamide, metsulfovax, metyltetraprole, milneb, mucochloric anhydride, myclozolin, /V-3,5-dichlorophenyl-succinimide, /V-3-nitrophenylitaconimide, natamycin, V-ethylmercurio-4-toluenesulfonanilide, nickel bis(dimethyldithiocarbamate), OCH, phenylmercury dimethyldithiocarbamate, phenylmercury nitrate, phosdiphen, prothiocarb; prothiocarb hydrochloride, pyracarbolid, pyrapropoyne, pyridachiometyl, pyridinitril, pyrisoxazole, pyroxychlor, pyroxyfur, quinacetol; quinacetol sulfate, quinazamid, quinconazole, quinofumelin, rabenzazole, salicylanilide, SSF-109, sultropen, tecoram, thiadifluor, thicyofen, thiochlorfenphim, thiophanate, thioquinox, tioxymid, triamiphos, triarimol, triazbutil, trichlamide, triclopyricarb, triflumezopyrim, urbacid, zarilamid, and any combinations thereof.

Additionally, the compounds described herein may be combined with other pesticides, including insecticides, nematocides, miticides, arthropodicides, bactericides or combinations thereof that are compatible with the compounds of the present disclosure in the medium selected for application, and not antagonistic to the activity of the present compounds to form pesticidal mixtures and synergistic mixtures thereof. The fungicidal compounds of the present disclosure may be applied in conjunction with one or more other pesticides to control a wider variety of undesirable pests. When used in conjunction with other pesticides, the presently claimed compounds may be formulated with the other pesticide(s), tank-mixed with the other pesticide(s) or applied sequentially with the other pesticide(s). Typical insecticides include, but are not limited to: 1,2-dichloropropane, abamectin, acephate, acetamiprid, acethion, acetoprole, acrinathrin, acrylonitrile, acynonapyr, afidopyropen, alanycarb, aldicarb, aldoxycarb, aldrin, allethrin, allosamidin, allyxycarb, alpha-cypermethrin, alpha-ecdysone, alpha-endosulfan, amidithion, aminocarb, amiton, amiton oxalate, amitraz, anabasine, athidathion, azadirachtin, azamethiphos, azinphos-ethyl, azinphos-methyl, azothoate, barium hexafluorosilicate, barthrin, bendiocarb, benfuracarb, bensultap, benzpyrimoxan, beta-cyfluthrin, beta-cypermethrin, bifenthrin, bioallethrin, bioethanomethrin, biopermethrin, bistrifluron, borax, boric acid, broflanilide, bromfenvinfos, bromocyclen, bromo-DDT, bromophos, bromophos-ethyl, bufencarb, buprofezin, butacarb, butathiofos, butocarboxim, butonate, butoxycarboxim, cadusafos, calcium arsenate, calcium polysulfide, camphechlor, carbanolate, carbaryl, carbofuran, carbon disulfide, carbon tetrachloride, carbophenothion, carbosulfan, cartap, cartap hydrochloride, chlorantraniliprole, chlorbicyclen, chlordane, chlordecone, chlordimeform, chlordimeform hydrochloride, chlorethoxyfos, chlorfenapyr, chlorfenvinphos, chlorfluazuron, chlormephos, chloroform, chloropicrin, chloroprallethrin, chlorphoxim, chlorprazophos, chlorpyrifos, chlorpyrifos-methyl, chlorthiophos, chromafenozide, cinerin I, cinerin II, cinerins, cismethrin, cloethocarb, closantel, clothianidin, copper acetoarsenite, copper arsenate, copper naphthenate, copper oleate, coumaphos, coumithoate, crotamiton, crotoxyphos, crufomate, cryolite, cyanofenphos, cyanophos, cyanthoate, cyantraniliprole, cyclaniliprole, cyclethrin, cycloprothrin, cyfluthrin, cyhalodiamide, cyhalothrin, cypermethrin. cyphenothrin, cyromazine, cythioate, DDT, decarbofuran, deltamethrin, demephion, demephion-O, demephion-S, demeton, demeton-methyl, demeton-O, demeton-O-methyl, demeton-S, demeton-S-methyl, demeton-S-methylsulphon, diafenthiuron, dialifos, diatomaceous earth, diazinon, dicapthon, dichlofenthion, dichlorvos, dicloromezotiaz, dicresyl, dicrotophos, dicyclanil, dieldrin, diflubenzuron, dilor, dimefluthrin, dimefox, dimetan, dimethoate, dimethrin, dimethylvinphos, dimetilan, dinex, dinex-diclexine, dinoprop, dinosam, dinotefuran, diofenolan, dioxabenzofos, dioxacarb, dioxathion, disulfoton, dithicrofos, d-limonene, DNOC, DNOC-ammonium, DNOC-potassium, DNOC-sodium, doramectin, ecdysterone, emamectin, emamectin benzoate, EMPC, empenthrin, endosulfan, endothion, endrin, EPN, epofenonane, eprinomectin, epsilon-metofluthrin, epsilon-momfluorothrin, esdepallethrine, esfenvalerate, etaphos, ethiofencarb, ethion, ethiprole, ethoate-methyl, ethoprophos, ethyl formate, ethyl-DDD, ethylene dibromide, ethylene dichloride, ethylene oxide, etofenprox, etrimfos, EXD, famphur, fenamiphos, fenazaflor, fenchlorphos, fenethacarb, fenfluthrin, fenitrothion, fenobucarb, fenoxacrim, fenoxycarb, fenpirithrin, fenpropathrin, fensulfothion, fenthion, fenthion-ethyl, fenvalerate, fipronil, flometoquin, flonicamid, fluazaindolizine, flubendiamide, flucofuron, flucycloxuron, flucythrinate, fluensulfone, flufenerim, flufenoxuron, flufenprox, flufiprole, fluhexafon, flupyradifurone, flupyrimin, fluvalinate, fluxametamide, fonofos, formetanate, formetanate hydrochloride, formothion, formparanate, formparanate hydrochloride, fosmethilan, fospirate, fosthietan, furathiocarb, furethrin, gamma-cyhalothrin, gamma-HCH, halfenprox, halofenozide, HCH, HEOD, heptachlor, heptafluthrin, heptenophos, heterophos, hexaflumuron, HHDN, hydramethylnon, hydrogen cyanide, hydroprene, hyquincarb, imidacloprid, imiprothrin, indoxacarb, iodomethane, IPSP, isazofos, isobenzan, isocarbophos, isocycloseram, isodrin, isofenphos, isofenphos-methyl, isoprocarb, isoprothiolane, isothioate, isoxathion, ivermectin, jasmolin I, jasmolin, jodfenphos, juvenile hormone I, juvenile hormone II, juvenile hormone, kappa-bifenthrin, kappa-tefluthrin, kelevan, kinoprene, lambda-cyhalothrin, lead arsenate, lepimectin, leptophos, lindane, lirimfos, lufenuron, lythidathion, malathion, malonoben, mazidox, mecarbam, mecarphon, menazon, meperfluthrin, mephosfolan, mercurous chloride, mesulfenfos, metaflumizone, methacrifos, methamidophos, methidathion, methiocarb, methocrotophos, methomyl, methoprene, methoxychlor, methoxyfenozide, methyl bromide, methyl isothiocyanate, methylchloroform, methylene chloride, metofluthrin, metolcarb, metoxadiazone, mevinphos, mexacarbate, milbemectin, milbemycin oxime, mipafox, mirex, molosultap, momfluorothrin, monocrotophos, monomehypo, monosultap, morphothion, moxidectin, naftalofos, naled, naphthalene, nicotine, nifluridide, nitenpyram, nithiazine, nitrilacarb, novaluron, noviflumuron, omethoate, oxamyl, oxazosulfyl, oxydemeton-methyl, oxydeprofos, oxydisulfoton, para-dichlorobenzene, parathion, parathion-methyl, penfluron, pentachlorophenol, permethrin, phenkapton, phenothrin, phenthoate, phorate, phosalone, phosfolan, phosmet, phosnichlor, phosphamidon, phosphine, phoxim, phoxim-methyl, pirimetaphos, pirimicarb, pirimiphos-ethyl, pirimiphos-methyl, potassium arsenite, potassium thiocyanate, pp′-DDT, prallethrin, precocene I, precocssene, precocene TIT, primidophos, profenofos, profluralin, promacyl, promecarb, propaphos, propetamphos, propoxur, prothidathion, prothiofos, prothoate, protrifenbute, pyflubumide, pyraclofos, pyrafluprole, pyrazophos, pyresmethrin, pyrethrin I, pyrethrin, pyrethrins, pyridaben, pyridalyl, pyridaphenthion, pyrifluquinazon, pyrimidifen, pyriminostrobin, pyrimitate, pyriprole, pyriproxyfen, quassia, quinalphos, quinalphos-methyl, quinothion, rafoxanide, resmethrin, rotenone, ryania, sabadilla, schradan, selamectin, silafluofen, silica gel, sodium arsenite, sodium fluoride, sodium hexafluorosilicate, sodium thiocyanate, sophamide, spinetoram, spinosad, spiromesifen, spiropidion, spirotetramat, sulcofuron, sulcofuron-sodium, sulfluramid, sulfotep, sulfoxaflor, sulfuryl fluoride, sulprofos, tau-fluvalinate, tazimcarb, TDE, tebufenozide, tebufenpyrad, tebupirimfos, teflubenzuron, tefluthrin, temephos, TEPP, terallethrin, terbufos, tetrachlorantraniliprole, tetrachloroethane, tetrachlorvinphos, tetramethrin, tetramethylfluthrin, tetraniliprole, theta-cypermethrin, thiacloprid, thiamethoxam, thicrofos, thiocarboxime, thiocyclam, thiocyclam oxalate, thiodicarb, thiofanox, thiometon, thiosultap, thiosultap-disodium, thiosultap-monosodium, thuringiensin, tioxazafen, tolfenpyrad, tralomethrin, transfluthrin, transpermethrin, triarathene, triazamate, triazophos, trichlorfon, trichlormetaphos-3, trichloronat, trifenofos, triflumezopyrim, triflumuron, trimethacarb, triprene, tyclopyrazoflor, vamidothion, vaniliprole, XMC, xylylcarb, zeta-cypermethrin, zolaprofos, and any combinations thereof.

Additionally, the compounds described herein may be combined with herbicides that are compatible with the compounds of the present disclosure in the medium selected for application, and not antagonistic to the activity of the present compounds to form pesticidal mixtures and synergistic mixtures thereof. The fungicidal compounds of the present disclosure may be applied in conjunction with one or more herbicides to control a wide variety of undesirable plants. When used in conjunction with herbicides, the presently claimed compounds may be formulated with the herbicide(s), tank-mixed with the herbicide(s) or applied sequentially with the herbicide(s). Typical herbicides include, but are not limited to: 4-CPA; 4-CPB; 4-CPP; 2,4-D; 3,4-DA; 2,4-DB; 3,4-DB; 2,4-DEB; 2,4-DEP; 3,4-DP; 2,3,6-TBA; 2,4,5-T; 2,4,5-TB; acetochlor, acifluorfen, aclonifen, acrolein, alachlor, allidochlor, alloxydim, allyl alcohol, alorac, ametridione, ametryn, amibuzin, amicarbazone, amidosulfuron, aminocyclopyrachlor, aminopyralid, amiprofos-methyl, amitrole, ammonium sulfamate, anilofos, anisuron, asulam, atraton, atrazine, azafenidin, azimsulfuron, aziprotryne, barban, BCPC, beflubutamid, beflubutamid-M, benazolin, bencarbazone, benfluralin, benfuresate, bensulfuron, bensulide, bentazone, benzadox, benzfendizone, benzipram, benzobicyclon, benzofenap, benzofluor, benzoylprop, benzthiazuron, bicyclopyrone, bifenox, bilanafos, bispyribac, bixlozone, borax, bromacil, bromobonil, bromobutide, bromofenoxim, bromoxynil, brompyrazon, butachlor, butafenacil, butamifos, butenachlor, buthidazole, buthiuron, butralin, butroxydim, buturon, butylate, cacodylic acid, cafenstrole, calcium chlorate, calcium cyanamide, cambendichlor, carbasulam, carbetamide, carboxazole chlorprocarb, carfentrazone, CDEA, CEPC, chlomethoxyfen, chloramben, chloranocryl, chlorazifop, chlorazine, chlorbromuron, chlorbufam, chloreturon, chlorfenac, chlorfenprop, chlorflurazole, chlorflurenol, chloridazon, chlorimuron, chlomitrofen, chloropon, chlorotoluron, chloroxuron, chloroxynil, chlorpropham, chlorsulfuron, chlorthal, chlorthiamid, cinidon-ethyl, cinmethylin, cinosulfuron, cisanilide, clacyfos, clethodim, cliodinate, clodinafop, clofop, clomazone, clomeprop, cloprop, cloproxydim, clopyralid, cloransulam, CMA, copper sulfate, CPMF, CPPC, credazine, cresol, cumyluron, cyanatryn, cyanazine, cycloate, cyclopyranil, cyclopyrimorate, cyclosulfamuron, cycloxydim, cycluron, cyhalofop, cyperquat, cyprazine, cyprazole, cypromid, daimuron, dalapon, dazomet, delachlor, desmedipham, desmetryn, di-allate, dicamba, dichlobenil, dichloralurea, dichlormate, dichlorprop, dichlorprop-P, diclofop, diclosulam, diethamquat, diethatyl, difenopenten, difenoxuron, difenzoquat, diflufenican, diflufenzopyr, dimefuron, dimepiperate, dimethachlor, dimethametryn, dimethenamid, dimethenamid-P, dimexano, dimidazon, dinitramine, dinofenate, dinoprop, dinosam, dinoseb, dinoterb, diphenamid, dipropetryn, diquat, disul, dithiopyr, diuron, DMPA, DNOC, DSMA, EBEP, eglinazine, endothal. epronaz. EPTC, erbon, esprocarb. ethalfluralin, ethametsulfuron, ethidimuron, ethiolate, ethofumesate, ethoxyfen, ethoxysulfuron, etinofen, etnipromid, etobenzanid, EXD, fenasulam, fenoprop, fenoxaprop, fenoxaprop-P, fenoxasulfone, fenquinotrione, fenteracol, fenthiaprop, fentrazamide, fenuron, ferrous sulfate, flamprop, flamprop-M, flazasulfuron, florasulam, florpyrauxifen, fluazifop, fluazifop-P, fluazolate, flucarbazone, flucetosulfuron, fluchloralin, flufenacet, flufenican, flufenpyr, flumetsulam, flumezin, flumiclorac, flumioxazin, flumipropyn, fluometuron, fluorodifen, fluoroglycofen, fluoromidine, fluoronitrofen, fluothiuron, flupoxam, flupropacil, flupropanate, flupyrsulfuron, fluridone, flurochloridone, fluroxypyr, flurtamone, fluthiacet, fomesafen, foramsulfuron, fosamine, furyloxyfen, glufosinate, glufosinate-P, glyphosate, halauxifen, halosafen, halosulfuron, haloxydine, haloxyfop, haloxyfop-P, hexachloroacetone, hexaflurate, hexazinone, imazamethabenz, imazamox, imazapic, imazapyr, imazaquin, imazethapyr, imazosulfuron, indanofan, indaziflam, iodobonil, iodomethane, iodosulfuron, iofensulfuron, ioxynil, ipazine, ipfencarbazone, iprymidam, isocarbamid, isocil, isomethiozin, isonoruron, isopolinate, isopropalin, isoproturon, isouron, isoxaben, isoxachlortole, isoxaflutole, isoxapyrifop, karbutilate, ketospiradox, lancotrione, lactofen, lenacil, linuron, MAA, MAMA, MCPA, MCPA-thiocthyl, MCPB, mecoprop, mecoprop-P, medinoterb, mefenacet, mefluidide, mesoprazine, mesosulfuron, mesotrione, metam, metamifop, metamitron, metazachlor, metazosulfuron, metflurazon, methabenzthiazuron, methalpropalin, methazole, methiobencarb, methiozolin, methiuron, methometon, methoprotryne, methyl bromide, methyl isothiocyanate, methyldymron, metobenzuron, metobromuron, metolachlor, metosulam, metoxuron, metribuzin, metsulfuron, molinate, monalide, monisouron, monochloroacetic acid, monolinuron, monuron, morfamquat, MSMA, naproanilide, napropamide, napropamide-M, naptalam, neburon, nicosulfuron, nipyraclofen, nitralin, nitrofen, nitrofluorfen, norflurazon, noruron, OCH, orbencarb, oriizo-dichlorobenzene, orthosulfamuron, oryzalin, oxadiargyl, oxadiazon, oxapyrazon, oxasulfuron, oxaziclomefone, oxyfluorfen, parafluron, paraquat, pebulate, pelargonic acid, pendimethalin, penoxsulam, pentachlorophenol, pentanochlor, pentoxazone, perfluidone, pethoxamid, phenisopham, phenmedipham, phenmedipham-ethyl, phenobenzuron, phenylmercury acetate, picloram, picolinafen, pinoxaden, piperophos, potassium arsenite, potassium azide, potassium cyanate, pretilachlor, primisulfuron, procyazine, prodiamine, profluazol, profluralin, profoxydim, proglinazine, prometon, prometryn, propachlor, propanil, propaquizafop, propazine, propham, propisochlor, propoxycarbazone, propyrisulfuron, propyzamide, prosulfalin, prosulfocarb, prosulfuron, proxan, prynachlor, pydanon, pyraclonil, pyraflufen, pyrasulfotole, pyrazolynate, pyrazosulfuron, pyrazoxyfen, pyribenzoxim, pyributicarb, pyriclor, pyridafol, pyridate, pyriftalid, pyriminobac, pyrimisulfan, pyrithiobac, pyroxasulfone, pyroxsulam, quinclorac, quinmerac, quinoclamine, quinonamid, quizalofop, quizalofop-P, rhodethanil, rimsulfuron, saflufenacil, S-metolachlor, sebuthylazine, secbumeton, sethoxydim, siduron, simazine, simeton, simetryn, SMA, sodium arsenite, sodium azide, sodium chlorate, sulcotrione, sulfallate, sulfentrazone, sulfometuron, sulfosulfuron, sulfuric acid, sulglycapin, swep, TCA, tebutam, tebuthiuron, tefuryltrione, tembotrione, tepraloxydim, terbacil, terbucarb, terbuchlor, terbumeton, terbuthylazine, terbutryn, tetrafluron, thenylchlor, thiazafluron, thiazopyr, thidiazimin, thidiazuron, thiencarbazone-methyl, thifensulfuron, thiobencarb, tiafenacil, tiocarbazil, tioclorim, tolpyralate, topramezone, tralkoxydim, triafamone, tri-allate, triasulfuron, triaziflam, tribenuron, tricamba, triclopyr, tridiphane, trietazine, trifloxysulfuron, trifludimoxazin, trifluralin, triflusulfuron, trifop, trifopsime, trihydroxytriazine, trimeturon, tripropindan, tritac, tritosulfuron, vemolate, and xylachlor.

The compounds and compositions of the subject invention are effective in the treatment, amelioration, prevention, elimination, delay of pathogens or confer resistance against pathogens which include but are not limited to viruses or viroids, bacteria, insects, nematodes, fungi, and the like. Viruses include any plant virus, for example, tobacco or cucumber mosaic virus, ringspot virus, necrosis virus, maize dwarf mosaic virus, etc. Specific fungal, fungal-like and viral pathogens for the major crops include: Phytophthora infestans, Phytophthora megasperma sp. glycinea, Macrophomina phaseolina, Rhizoctonia solani, Sclerotinia sclerotiorum, Fusarium oxysporum, Diaporthe phaseolorum var. sojae (Phomopsis sojae), Diaporthe phaseolorum var. caulivora, Sclerotium rolfsii, Cercospora kikuchii, Cercospora sojina, Peronospora manshurica, Colletotrichum dematium (Colletotichum truncatum), Corynespora cassiicola, Septoria glycines, Phyllosticta sojicola, Alternaria alternata, Pseudomonas syringae p.v. glycinea, Xanthomonas campestris p.v. phaseoli, Microsphaera diffusa, Fusarium semitectum, Phialophora gregata, Soybean mosaic virus, Glomerella glycines, Tobacco Ring spot virus, Tobacco Streak virus, Phakopsora pachyrhizi, Pythium aphanidermatum, Pythium ultimum, Pythium debaryanum, Tomato spotted wilt virus, Heterodera glycines Fusarium solani; Albugo Candida, Alternaria brassicae, Leptosphaeria maculans, Rhizoctonia solani, Sclerotinia sclerotiorum, Mycosphaerella brassiccola, Pythium ultimum, Peronospora parasitica, Fusarium roseum, Alternaria alternata; Clavibacter michigdnensis subsp. insidiosum, Pythium ultimum, Pythium irregulare, Pythium splendens, Pythium debaryanum, Pythium aphanidermatum, Phytophthora megasperma, Peronospora trifoliorum, Phoma medicaginis var. medicaginis, Cercospora medicaginis, Pseudopeziza medicaginis, Leptotrochila medicaginis, Fusarium, Xanthomonas campestris p.v. alfalfae, Aphanomyces euteiches, Stemphylium herbarum, Stemphylium alfalfae; Zymoseptoria tritici, Pseudomonas syringae p.v. atrofaciens, Urocystis agropyri, Xanthomonas campestris p.v. translucens, Pseudomonas syringae p.v. syringae, Alternaria alternata, Cladosporium herbarum, Fusarium graminearum, Fusarium avenaceum, Fusarium culmorum, Ustilago tritici, Ascochyta tritici, Cephalosporium gramineum, Collotetrichum graminicola, Erysiphe graminis f.sp. tritici, Puccinia triticina, Puccinia graminis f.sp. tritici, Puccinia recondita f.sp. tritici, Puccinia striiformis, Pyrenophora tritici-repentis, Septoria nodorum, Septoria tritici, Septoria avenae, Pseudocercosporella herpotrichoides, Rhizoctonia solani, Rhizoctonia cerealis, Gaeumannomyces graminis var. tritici, Pythium aphanidermatum, Pythium arrhenomanes, Pythium ultimum, Bipolaris sorokiniana, Barley Yellow Dwarf Virus, Brome Mosaic Virus, Soil Borne Wheat Mosaic Virus, Wheat Streak Mosaic Virus, Wheat Spindle Streak Virus, American Wheat Striate Virus, Claviceps purpurea, Tilletia tritici, Tilletia laevis, Ustilago tritici, Tilletia indica, Rhizoctonia solani, Pythium arrhenomanes, Pythium gramicola, Pythium aphanidermatum, High Plains Virus, European wheat striate virus; Plasmophora halstedii, Sclerotinia sclerotiorum, Aster Yellows, Septoria helianthi, Phomopsis helianthi, Alternaria helianthi, Alternaria zinniac, Botrytis cinerea, Phoma macdonaldii, Macrophomina phaseolina, Erysiphe dehor acearum, Rhizopus oryzae, Rhizopus arrhizus, Rhizopus stolonifer, Puccinia helianthi, Verticillium dahliae, Erwinia carotovorum p.v. carotovora, Cephalosporium acremonium, Phytophthora cryptogea, Albugo tragopogonis; Fusarium moniliforme var. subglutinans, Erwinia stewartii, Fusarium verticilloides, Fusarium moniliforme, Gibberella zeae (Fusarium graminearum), Stenocarpella maydis (Diplodia maydis), Pythium irregulare, Pythium debaryanum, Pythium graminicola, Pythium splendens, Pythium ultimum, Pythium aphanidermatum, Aspergillus flavus, Bipolaris maydis O, T (Cochliobolus heterostrophus), Helminthosporium carbonum I, II & III (Cochliobolus carbonum), Exserohilum turcicum I, II & III, Helminthosporium pedicellatum, Physoderma maydis, Phyllosticta maydis, Kabatiella maydis, Cercospora sorghi, Ustilago maydis, Puccinia sorghi, Puccinia polysora, Macrophomina phaseolina, Penicillium oxalicum, Nigrospora oryzac, Cladosporium herbarum, Curvularia lunata, Curvularia inaequalis, Curvularia pallescens, Clavibacter michiganense subsp. nebraskense, Trichoderma viride, Maize Dwarf Mosaic Virus A & B, Wheat Streak Mosaic Virus, Maize Chlorotic Dwarf Virus, Claviceps sorghi, Pseudomonas avenae, Erwinia chrysanthemi pv. zea, Erwinia carotovora, Corn stunt spiroplasma, Diplodia macrospora, Sclerophthora macrospora, Peronosclerospora sorghi, Peronosclerospora philippinensis, Peronosclerospora maydis, Peronosclerospora sacchari, Sphacelotheca reiliana, Physopella zeae, Cephalosporium maydis, Cephalosporium acremonium, Maize Chlorotic Mottle Virus, High Plains Virus, Maize Mosaic Virus, Maize Rayado Fino Virus, Maize Streak Virus, Maize Stripe Virus, Maize Rough Dwarf Virus; Exserohilum turcicum, Colletotrichum graminicola (Glomerella graminicola), Cercospora sorghi, Gloeocercospora sorghi, Ascochyta sorghina, Pseudomonas syringae p.v. syringae, Xanthomonas campestris p.v. holcicola, Pseudomonas andropogonis, Puccinia purpurea, Macrophomina phaseolina, Periconia circinata, Fusarium moniliforme, Alternaria alternata, Bipolaris sorghicola, Helminthosporium sorghicola, Curvularia lunata, Phoma insidiosa, Pseudomonas avenae (Pseudomonas alboprecipitans), Ramulispora sorghi, Ramulispora sorghicola, Phyllachara sacchari, Sporisorium reilianum (Sphacelotheca reiliana), Sphacelotheca cruenta, Sporisorium sorghi, Sugarcane mosaic H, Maize Dwarf Mosaic Virus A & B, Claviceps sorghi, Rhizoctonia solani, Acremonium strictum, Sclerophthora macrospora, Peronosclerospora sorghi, Peronosclerospora philippinensis, Sclerospora graminicola, Fusarium graminearum, Fusarium oxysporum, Pythium arrhenomanes, Pythium graminicola; Rice Magnaporthe grisca, Rhizoctonia solani, etc. Bacterial infections may be caused by at least one of: Clavibacter michiganensis, Agrobacterium tumefaciens, Erwinia amylovora, Pseudomonas syringae pv. lachrymans, Pseudomonas syringae pv. tomato, Streptomyces scabies, Xanthomonas campestris pv. campestris and Xanthomonas capestris pv. vesicatoria.

The present invention provides a method of treating a locus against pest infestation comprising applying an effective amount of any one of the compositions or combinations described herein to the locus so as to thereby treat the locus against pest infestation.

The present invention also provides a method for treating a plant or a plant material against pest infestation comprising applying an effective amount of any one of the compositions or combinations described herein to the plant, the plant material, or the vicinity of the plant or plant material so as to thereby treat the plant or plant material against pest infestation.

The present invention also provides a method for controlling and/or preventing of fungal attack on soil, plant, root, foliage, seed, locus of the fungus, or a locus where fungal infestation is to be prevented, wherein the method comprises applying a fungicidally effective amount of any one of the compositions or combinations described herein to the soil, plant, roots, foliage, seed, locus of the fungus, or locus in which the infestation is to be prevented so as to thereby control and/or prevent fungal attack.

In some embodiments, the composition or combination is diluted prior to application.

The present invention also provides a method for treating a plant or a plant material against pest infestation comprising (i) obtaining any one of the composition or combination described herein, (ii) diluting the composition or combination, and (iii) applying an effective amount of the diluted composition or combination to the plant, the plant material, or the vicinity of the plant or plant material so as to thereby treat the plant or plant material against pest infestation.

The present invention also provides a method for treating a plant or a plant material against pest infestation comprising (i) obtaining any one of the composition or combination described herein, (ii) diluting the composition or combination, and (iii) applying an effective amount of the diluted composition or combination to the plant, the plant material, or the vicinity of the plant or plant material so as to thereby treat the plant or plant material against pest infestation.

The present invention also provides a method for controlling and/or preventing of fungal attack on soil, plant, root, foliage, seed, locus of the fungus, or a locus where fungal infestation is to be prevented, wherein the method comprises (i) obtaining any one of the composition or combination described herein, (ii) diluting the composition or combination, and (iii) applying an effective amount of the diluted composition or combination to the soil, plant, roots, foliage, seed, locus of the fungus, or locus in which the infestation is to be prevented so as to thereby control and/or prevent fungal attack.

In some embodiments, the concentration of the compound of Formula T in the obtained composition or combination is 0.03-1000 g/L. In some embodiments, the concentration of the compound of Formula I in the obtained composition or combination is 0.05-1000 g/L. In some embodiments, the concentration of the compound of Formula I in the obtained composition or combination is 0.1-1000 g/L. In some embodiments, the concentration of the compound of Formula I in the obtained composition or combination is 10-1000 g/L. In some embodiments, the concentration of the compound of Formula I in the obtained composition or combination is 50-1000 g/L. In some embodiments, the concentration of the compound of Formula I in the obtained composition or combination is 100-1000 g/L. In some embodiments, the concentration of the compound of Formula I in the obtained composition or combination is 200-1000 g/L. In some embodiments, the concentration of the compound of Formula I in the obtained composition or combination is 300-1000 g/L. In some embodiments, the concentration of the compound of Formula I in the obtained composition or combination is 400-1000 g/L. In some embodiments, the concentration of the compound of Formula I in the obtained composition or combination is 500-1000 g/L. In some embodiments, the concentration of the compound of Formula I in the obtained composition or combination is 600-1000 g/L. In some embodiments, the concentration of the compound of Formula I in the obtained composition or combination is 700-1000 g/L. In some embodiments, the concentration of the compound of Formula I in the obtained composition or combination is 800-1000 g/L. In some embodiments, the concentration of the compound of Formula I in the obtained composition or combination is 900-1000 g/L.

In some embodiments, the combination or composition is applied to the plant. In some embodiments, the combination or composition is applied to propagation material of a plant. In some embodiments, the combination or composition is applied to the vicinity of a plant. In some embodiments, the combination or composition is applied to the vicinity of a propagation material of a plant. In some embodiments, the combination or composition is applied to soil. In some embodiments, application of the combination or composition to soil is effective to control pest(s) that are resident there. In some embodiments, the combination or composition is applied to seed of the plant. In some embodiments, the combination or composition is applied to foliage of the plant.

In some embodiments, the combination or composition is applied in the early stages of the crop cycle. In some embodiments, the combination or composition is applied pre-sowing of the crop. In some embodiments, the combination or composition is applied post-sowing of the crop.

In some embodiments, the combination or composition is applied to harvested produce. In some embodiments, application of the combination or composition to harvested produce is effective to prevent storage losses.

In some embodiments, the combination or composition is applied at a rate from 1 g/ha to 10000 g/ha of the compound of Formula I or an agriculturally acceptable salt thereof. In some embodiments, the combination or composition is applied at a rate from 10 g/ha to 10000 g/ha of the compound of Formula I or an agriculturally acceptable salt thereof. In some embodiments, the combination or composition is applied at a rate from 50 to 5000 g/ha of the compound of Formula I or an agriculturally acceptable salt thereof. In some embodiments, the combination or composition is applied at a rate from 100 to 2000 g/ha of the compound of Formula I or an agriculturally acceptable salt thereof.

The present invention also provides use of any one of the compositions or combinations described herein for treating a locus against pest infestation.

In some embodiments, the combination or composition is used to protect the locus against pest infestation. In some embodiments, the combination or composition is used to prevent pest infestation from affecting the locus, protect the locus from pest infestation, delay the onset of pest infestation affecting the locus, and/or confer resistance to the locus against pest infestation.

In some embodiments, the combination or composition is used to control pest infestation affecting the locus. In some embodiments, the combination or composition is used to combat the pest infestation affecting the locus, reduce the severity of the pest infestation affecting the locus, cure the pest infestation affecting the locus, ameliorate the pest infestation affecting the locus, inhibit the pest infestation affecting the locus, and/or eliminate the pest infestation affecting the locus.

The present invention also provides use of any one of the compositions or combinations described herein for treating a plant or a plant material against pest infestation.

The present invention also provides use of any one of the compositions or combinations described herein for controlling and/or preventing of fungal attack on soil, plant, root, foliage, seed, locus of the fungus, or a locus where fungal infestation is to be prevented.

The present invention also provides any one of the compositions or combinations described herein for use in treating a locus against pest infestation.

The present invention also provides any one of the compositions or combinations described herein for use in treating a plant or a plant material against pest infestation.

The present invention also provides any one of the compositions or combinations described herein for use in controlling and/or preventing of fungal attack on soil, plant, root, foliage, seed, locus of the fungus, or a locus where fungal infestation is to be prevented.

The present invention also provides a method of preparing a crude extract comprising a compound of Formula I or agriculturally acceptable salt thereof comprising the steps of:
(i) producing a conditioned culture medium of a fungus,
(ii) providing nitrogen-limiting conditions,
(iii) filtering the medium through a resin, and
(iv) eluting the adsorbed material from the resin with a polar solvent.

In some embodiments, the fungus is Pseudozyma aphidis.

In some embodiments, the compound of Formula I is selected from the group consisting of (E)-2-octyl-2-pentenedioic acid, (Z)-2-octyl-2-pentenedioic acid, and (E/Z)-2-octyl-2-pentenedioic acid.

In some embodiments, the polar solvent is selected from the group consisting of alcohol, dimethylformamide, dimethylsulfoxide, acetonitrile, propylene carbonate, acetone, and any combination thereof.

In some embodiments, the polar solvent is an alcohol. In a specific embodiment, the alcohol is methanol. In a specific embodiment, the alcohol is ethanol.

In some embodiments, the resin is selected from the group consisting of amberlite resin, silica gel, alumina resin and any combination thereof. In a specific embodiment, the resin is amberlite resin.

The present invention also provides a process for extracting the compound of Formula I from a fungus comprising:
(i) producing a conditioned culture medium of the fungus,
(ii) providing nitrogen-limiting conditions,
(iii) filtering the medium through a resin,
(iv) eluting the adsorbed material from the resin with a polar solvent, and
(v) isolating and purifying the compound of Formula (1) from the eluted material.

In some embodiments, the fungus is Pseudozyma aphidis.

In some embodiments, the polar solvent is selected from the group consisting of alcohol, dimethylformamide, dimethylsulfoxide, acetonitrile, propylene carbonate, acetone, and any combination thereof.

In some embodiments, the polar solvent is an alcohol. In a specific embodiment, the alcohol is methanol. In a specific embodiment, the alcohol is ethanol.

In some embodiments, the resin is selected from the group consisting of amberlite resin, silica gel, alumina resin and any combination thereof. In a specific embodiment, the resin is amberlite resin.

The process of producing a conditioned culture medium of Pseudozyma aphidis may be produced in accordance with WO 2011/151819, which is hereby incorporated by reference.

In some embodiments, the nitrogen source may include but is not limited to yeast extract, soy flour and tryptone. In some embodiments, the nitrogen-limiting condition is provided by using a low amount of yeast extract, soy flour and/or tryptone in the medium.

In some embodiments, the amount of yeast extract in the medium is 1.5 g/L or less. In some embodiments, the amount of yeast extract in the medium is 1 g/L or less. In some embodiments, the amount of yeast extract in the medium is 0.5 g/L or less.

In some embodiment, the amount of nitrogen in the medium is less than 1 g/L. In some embodiment, the amount of nitrogen in the medium is less than 0.5 g/L. In some embodiment, the amount of nitrogen in the medium is less than 0.4 g/L. In some embodiment, the amount of nitrogen in the medium is less than 0.3 g/L. In some embodiment, the amount of nitrogen in the medium is less than 0.2 g/L. In some embodiment, the amount of nitrogen in the medium is less than 0.1 g/L. In some embodiment, the amount of nitrogen in the medium is less than 0.05 g/L. In some embodiment, the amount of nitrogen in the medium is 0.05-0.1 g/L. In some embodiment, the amount of nitrogen in the medium is 0.05-0.15 g/L.

Nitrogen-limiting conditions in the production stage were shown to be essential for production of the compounds of Formula (I) in high concentration.

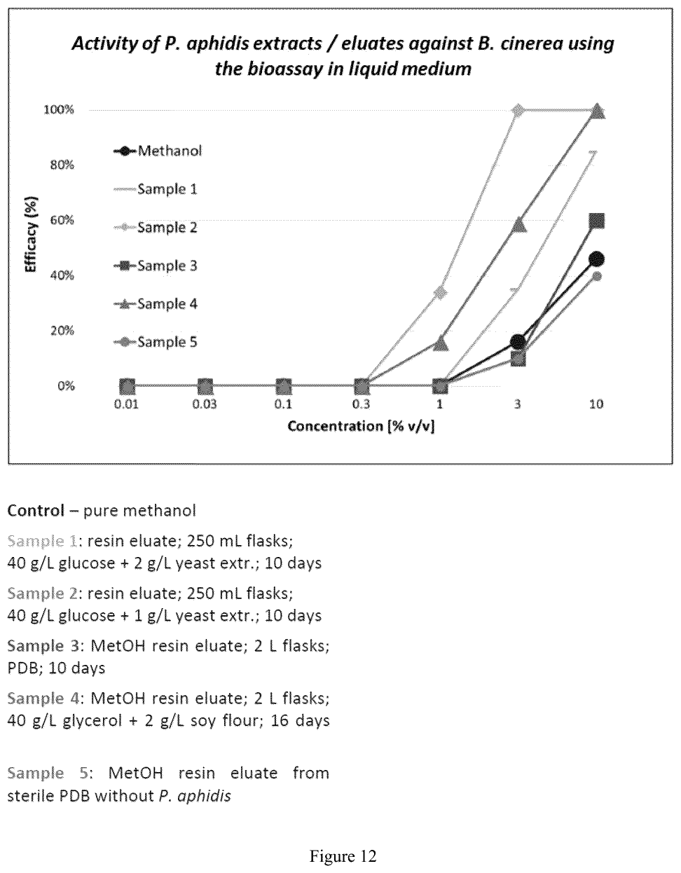

According to the specifications, yeast extract has the total nitrogen content of 9-12%, while in the media in which 0.5-1 g/L yeast extract were used, the final concentration of nitrogen in the medium was approximately 0.05-0.1 g/L. However, nitrogen content in the medium should not be the only criteria and carbon to nitrogen ratio should also be considered. According to the experiments (see FIGS. 12-14), the optimal C:N ratio for glucose as well as glycerol to yeast extract was in the range of 80:1 to 40:1 (gram glucose/glycerol per g YE). Potato dextrose broth (PDB) has a C:N ratio of 10:1 where glucose is the C-source. Therefore, if the C:N ratio of the media compared is significantly different than C:N in PDB. (4- to 8-fold higher ratio in the media) and that this resulted in improved production of antifungal compounds in our (nitrogen limited) media vs. PDB.

In some embodiments, the cultured medium may be filtered through a resin. The resin serves as an inert carrier, which binds various metabolites via weak hydrophobic interactions and therefore removes them from solution. This effectively reduces the inhibitory effect of potentially toxic compounds produced during the bioprocess and enables de novo biosynthesis of these metabolites. The resin may include amberlite resin, silica gel and alumina resin. An amberlite resin is an insoluble macroporous polystyrene based resin in the form of small (0.25-0.5 mm radius) microbeads. The beads are porous, thus providing a large surface area on and inside them.

The material adsorbed onto the resin, which may include the compound of Formula (T), may be eluted from the resin with a polar solvent. Suitable polar solvents may include alcohols such as for example methanol, and ethanol; dimethylformamide, dimethylsulfoxide, acetonitrile, propylene carbonate and acetone.

The isolating and purifying may be accomplished using methods known in the art, such as for example, but not limited to, flash chromatography and preparative HPLC.

The present invention also provides a process for preparation of compound of formula (Ia) comprising:

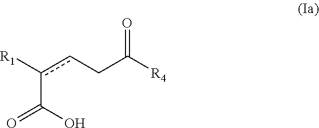

a) reacting a compound of formula (II) with compound of formula (HI) in the presence of base and optionally in the presence of organic solvent

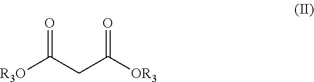

R₁X (III)
to form a compound of formula (IV),

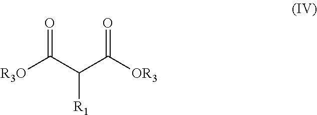

b) reacting the compound of formula (IV) with a compound of formula (V) in the presence of base and optionally in the presence of organic solvent

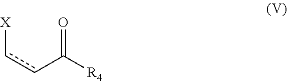

to form a compound of formula (VI), and

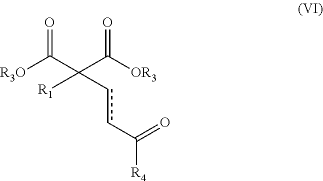

c) decarboxylatinig the compound of formula (VI) in the presence of catalyst and optionally in the presence of organic solvent to form the compound of formula (Ia),
wherein
R₁ is a C₁-C₁₂ alkyl optionally substituted with one or more substituents independently selected from the group consisting of halogen, C(O)R, CN, NH₂, —NH(C₁-C₄ alkyl), —(C₁-C₄ alkyl)—NH₂, —N(C₂-C₄ alkyl)(C₂-C₄ alkyl), OR. —S(O)₂(C₁-C₄, alkyl), —S(O)(C₁-C₄ alkyl), C(O)OH and C(O)OR;
R₃ is hydrogen or C₁-C₄ alkyl;
R₄ is OH, —O⁻, —NH₂, —NH—NH₂, —NH(C₁-C₁₂ alkyl), —N(C₂-C₄ alkyl)(C₂-C₄ alkyl), —O(C₁-C₁₂ alkyl), halogen, SR₃, or S(O)R₃;
X is bromine, chlorine or fluorine; and
R is independently H or an optionally substituted C₁-C₁₂ alkyl group.

The present invention also provides a process for preparation of compound of formula (Ia) comprising:

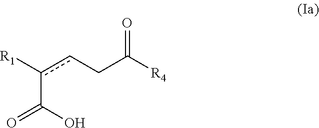

a) reacting a compound of formula (HI) with compound of formula (III) in the presence of base and optionally in the presence of organic solvent

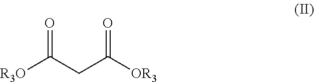

R₁X (III)
to form a compound of formula (IV),

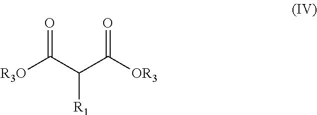

b) reacting the compound of formula (IV) with a compound of formula (V) in the presence of base and optionally in the presence of organic solvent

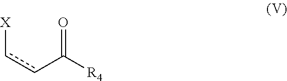

to form a compound of formula (VI), and

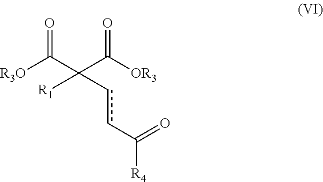

c) decarboxylating the compound of formula (VI) in the presence of catalyst and optionally in the presence of organic solvent to form the compound of formula (Ia),
wherein
R₁ is a C₁-C₁₂ alkyl optionally substituted with one or more substituents independently selected from the group consisting of halogen, CN, NH₂, —NH(C₁-C₄ alkyl), —(C₁-C₄ alkyl)—NH₂, —N(C₂-C₄ alkyl)(C₂-C₄ alkyl), OR, —S(O)₂(C₁-C₄ alkyl), —S(O)(C₁-C₄ alkyl), C(O)OH and C(O)OR;
R₃ is hydrogen or C₁-C₄ alkyl;
R₄ is OH, —O—, —NH₂, —NH—NH₂, —NH(C₁-C₁₂ alkyl), —N(C₂-C₄ alkyl)(C₂-C₄ alkyl), —O(C₁-C₁₂ alkyl), halogen, SR₃, or S(O)R₃;
X is bromine, chlorine or fluorine; and
R is independently H or an optionally substituted C₁-C₁₂ alkyl group.

The present invention also provides a process for preparation of compound of formula (Ia) comprising:

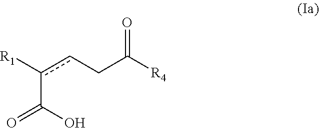

a) reacting a compound of formula (11) with compound of formula (III) in the presence of base and optionally in the presence of organic solvent

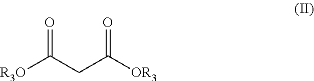

R₁X (III)
to form a compound of formula (IV),

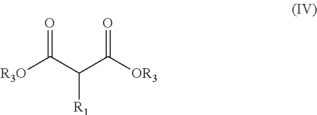

b) reacting the compound of formula (IV) with a compound of formula (V) in the presence of base and optionally in the presence of organic solvent

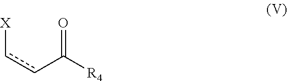

to form a compound of formula (VI), and

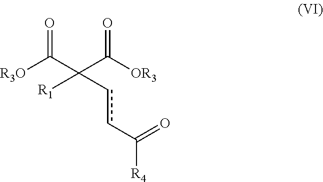

c) decarboxylating the compound of formula (VI) in the presence of catalyst and optionally in the presence of organic solvent to form the compound of formula (Ia),
wherein
R₁ is a C₁-C₁₂ alkyl optionally substituted with one or more substituents independently selected from the group consisting of halogen, CN, NH₂, —NH(C₁-C₄ alkyl), —(C₁-C₄ alkyl)—NH₂, —N(C₂-C₄ alkyl)(C₂-C₄ alkyl), OR, —S(O)₂(C₁-C₄ alkyl), —S(O)(C₁-C₄ alkyl), C(O)OH and C(O)OR;
R₃ is hydrogen or C₁-C₄ alkyl;
R₄ is OH, —O(C₁-C₁₂ alkyl), halogen, —NH(C₁-C₁₂ alkyl), SR₃, or S(O)R₃;
X is bromine, chlorine or fluorine; and
R is independently H or an optionally substituted C₁-C₁₂ alkyl group.

The present invention also provides a process for preparation of compound of formula (Ia) comprising:

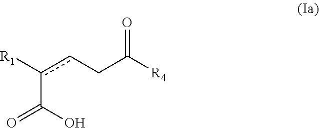

a) reacting a compound of formula (II) with compound of formula (III) in the presence of base and optionally in the presence of organic solvent

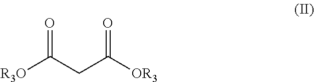

R₁X (III)
to form a compound of formula (TV),

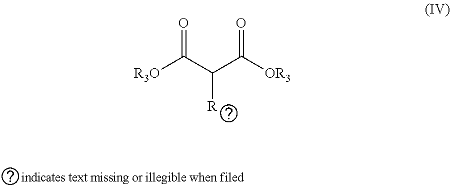

b) reacting the compound of formula (IV) with a compound of formula (V) in the presence of base and optionally in the presence of organic solvent

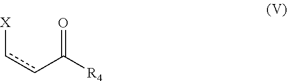

to form a compound of formula (VI), and

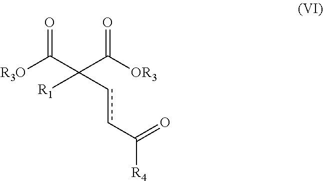

c) decarboxylating the compound of formula (VI) in the presence of catalyst and optionally in the presence of organic solvent to form the compound of formula (Ia),
wherein
R₁ is a C₁-C₁₂ alkyl optionally substituted with one or more substituents independently selected from the group consisting of halogen, CN, NH₂, C₁-C₄ alkylamino, C₂-C₄ dialkylamino, OR, C₁-C₄ alkylsulfonyl, C₁-C₄ alkylsulfinyl, C(O)OH, C(O)OR;
R₃ is hydrogen or C₁-C₄ alkyl;
R₄ is OH, C₁-C₁₂ alkoxy, halogen, C₁-C₁₂ alkyl amino, SR₃, or S(O)R₃;
X is bromine, chlorine or fluorine; and
R is H or an optionally substituted C₁-C₁₂ alkyl group.

In some embodiments, the two R₃ are the same as each other. In some embodiments, the two R₃ are different from each other.

The present invention also provides a process for preparation of compound of formula (Ia) comprising:

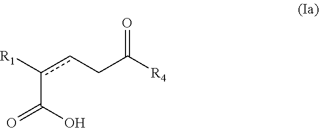

a) reacting a compound of formula (II) with compound of formula (III) in the presence of base and optionally in the presence of organic solvent

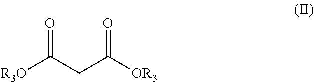

R₁X (III)
to form a compound of formula (IV),

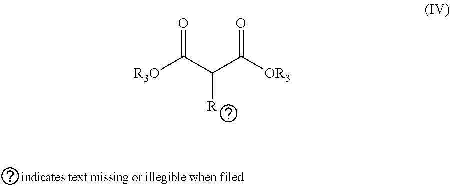

b) reacting the compound of formula (IV) with a compound of formula (V) in the presence of base and optionally in the presence of organic solvent

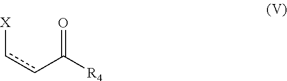

to form a compound of formula (VI), and

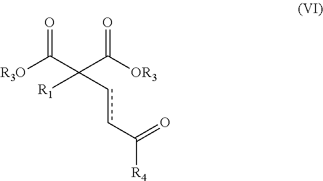

c) decarboxylating the compound of formula (VI) in the presence of catalyst and optionally in the presence of organic solvent to form the compound of formula (Ia),
wherein
R₁ is a C₁-C₁₂ alkyl optionally substituted with one or more substituents independently selected from the group consisting of halogen, CN, NH₂, C₁-C₄ alkylamino, C₂-C₄ dialkylamino, OR, C₁-C₄ alkylsulfonyl, C₁-C₄ alkylsulfinyl, C(O)OH, C(O)OR;
R₃ is hydrogen or C₁-C₄ alkyl;
R₄ is OH, C₁-C₁₂ alkoxy, halogen, C₁-C₁₂ alkyl amino, SR₃, or S(O)R₃;
X is bromine, chlorine or fluorine; and
R is H or an optionally substituted C₁-C₁₂ alkyl group.

In some embodiments, R₁ is C₆-C₁₂ alkyl. In some embodiments, R₁ is octyl.

In some embodiments, the two R₃ are the same as each other. In some embodiments, the two R₃ are different from each other.

In some embodiments, R₄ is OH.

In some embodiments, the base is selected from the group consisting of alkali metal salt of hydrides, alkali metal salt of carbonates, alkali metal salt of alkoxides, alkali metal salt of C₁-C₄ alkyls, and mixtures thereof.

In some embodiments, the base is an alkali metal salt of hydride. In some embodiments, the base is sodium hydride.

In some embodiments, the organic solvent is selected from the group consisting of C₁-C₄ alcohols, ethers, C₁-C₄ alkyl esters, lactones, amides, sulfoxides, aliphatic and aromatic carbohydrates, and the mixtures thereof.

In some embodiments, the organic solvent is a cyclic ether. In some embodiments, the organic solvent is tetrahydrofuran.

In some embodiments, the process is performed in the absence of organic solvent.

In some embodiments, the process is performed in the presence of organic solvent.

In some embodiments, the compound of formula (Ia) is (Z)-isomer and is substantially free of (E)-isomer of compound of formula (Ia).

In some embodiments, more than 95% of the compound of Formula (Ta) is (Z)-isomer.

In some embodiments, the compound of formula (Ia) is (E)-isomer and is substantially free of (Z)-isomer of compound of formula (Ia).

In some embodiments, more than 95% of the compound of Formula (Ia) is (E)-isomer.

In some embodiments, the compound of formula (Ia) is (E)-isomer, (Z)-isomer or a mixture thereof.

In some embodiments, the catalyst is an inorganic acid. In some embodiments, the inorganic acid is HCl, H₂SO₄, or a mixture thereof.

In some embodiments, the catalyst is an organic acid. In some embodiments, the organic acid is selected from the group consisting of carboxylic acids, amines and mixtures thereof.

The present invention also provides a process for preparing 2-octyl-2-pentenedioic acid or a salt thereof, wherein the process comprises:
a) reacting diethyl malonate with 1-halooctane to obtain diethyl 2-octylmalonate or a salt thereof;
b) reacting the obtained diethyl 2-octylmalonate with ethyl-3-haloacrylate to form triethyl-undec-1-ene-1,3,3-tricarboxylate or a salt thereof; and
c) converting the formed triethyl-undec-I-ene-1,3,3-tricarboxylate to obtain 2-octyl-2-pentenedioic acid.

In some embodiments, the obtained 2-octyl-2-pentenedioic acid is (E)-2-octyl-2-pentenedioic acid, (Z)-2-octyl-2-pentenedioic acid, (E/Z)-2-octyl-2-pentenedioic acid, or any mixture thereof.

In some embodiments, the obtained 2-octyl-2-pentenedioic acid is (Z)-2-octyl-2-pentenedioic acid.

In some embodiments, the obtained 2-octyl-2-pentenedioic acid is (E)-2-octyl-2-pentenedioic acid.

In some embodiments, the obtained 2-octyl-2-pentenedioic acid is (Z)-2-octyl-2-pentenedioic acid and is substantially free of (E)-2-octyl-2-pentenedioic acid.

In some embodiments, more than 95% of the obtained 2-octyl-2-pentenedioic acid is (Z)-2-octyl-2-pentenedioic acid.

In some embodiments, the obtained 2-octyl-2-pentenedioic acid is (E)-2-octyl-2-pentenedioic acid and is substantially free of (Z)-2-octyl-2-pentenedioic acid.

In some embodiments, more than 95% of the obtained 2-octyl-2-pentenedioic acid is (E)-2-octyl-2-pentenedioic acid.

In some embodiments, the 1-halooctane is selected from the group consisting of 1-bromooctane, 1-chlorooctane, and 1-fluorooctane.

In some embodiments, the ethyl-3-haloacrylate is selected from the group consisting of ethyl-3-bromoacrylate, ethyl-3-chloroacrylate, and ethyl-3-fluoroacrylate.

In some embodiments, step a) is carried out in the presence of a base.

In some embodiments, the base is selected from the group consisting of alkali metal salt of hydrides, alkali metal salt of carbonates, alkali metal salt of alkoxides, alkali metal salt of C₁-C₄ alkyls and the mixtures thereof.

In some embodiments, the base is an alkali metal salt of hydride. In some embodiments, the base is sodium hydride.

In some embodiments, the conversion in step c) is carried out in the presence of an inorganic acid. In some embodiments, the inorganic acid is selected from the group consisting of HCl, H₂SO₄, and a mixture thereof. In some embodiments, the conversion in step c) is carried out in the presence of an organic acid. In some embodiments, the organic acid is selected from the group consisting of carboxylic acids, amines, and mixtures thereof.

In some embodiments, the conversion of step c) is carried out at pH of about 1-4.

The present invention also provides use of(i) the crude extract comprising a compound of Formula I or agriculturally acceptable salt thereof prepared using the method described herein or (ii) the compound of Formula I extracted using the process described herein for treating a locus against pest infestation.

The present invention also provides use of(i) the crude extract comprising a compound of Formula I or agriculturally acceptable salt thereof prepared using the method described herein or (ii) the compound of Formula I extracted using the process described herein for treating a plant or a plant material against pest infestation.

The present invention also provides use of (i) the crude extract comprising a compound of Formula I or agriculturally acceptable salt thereof prepared using the method described herein or (ii) the compound of Formula I extracted using the process described herein for controlling and/or preventing of fungal attack on soil, plant, root, foliage, seed, locus of the fungus, or a locus where fungal infestation is to be prevented.

The present invention also provides a method of treating a locus against pest infestation comprising (i) preparing a crude extract comprising a compound of Formula I or agriculturally acceptable salt thereof using the method described herein or (ii) extracting the compound of Formula I using the process described herein, and applying an effective amount of the crude extract or the compound of Formula T to the locus so as to thereby treat the locus against pest infestation.

The present invention also provides a method of treating a plant or a plant material against pest infestation comprising (i) preparing a crude extract comprising a compound of Formula I or agriculturally acceptable salt thereof using the method described herein or (ii) extracting the compound of Formula I using the process described herein, and applying an effective amount of the crude extract or the compound of Formula to the plant, the plant material, or the vicinity of the plant or plant material so as to thereby treat the plant or plant material against pest infestation.

The present invention also provides a method of controlling and/or preventing of fungal attack on soil, plant, root, foliage, seed, locus of the fungus, or a locus where fungal infestation is to be prevented comprising (i) preparing a crude extract comprising a compound of Formula I or agriculturally acceptable salt thereof using the method described herein or (ii) extracting the compound of Formula I using the process described herein and applying an effective amount of the crude extract or the compound of Formula I to the soil, plant, roots, foliage, seed, locus of the fungus, or locus in which the infestation is to be prevented so as to thereby control and/or prevent fungal attack.

The present invention provides a method for treating a plant or a plant material against pest infestation comprising applying an effective amount of at least one compound of Formula I as described herein or an agricultural acceptable salt thereof to the plant, the plant material, or the vicinity of the plant or plant material so as to thereby treat the plant or plant material against pest infestation, wherein the compound of Formula I is produced synthetically.

The present invention provides a method for treating a plant or a plant material against pest infestation comprising applying an effective amount of at least one compound of Formula I as described herein or an agricultural acceptable salt thereof to the plant, the plant material, or the vicinity of the plant or plant material so as to thereby treat the plant or plant material against pest infestation, wherein the compound of Formula I is applied free of fungal material from Pseudozyma aphidis.

The present invention provides a method for treating a plant or a plant material against pest infestation comprising applying an effective amount of at least one compound of Formula I as described herein or an agricultural acceptable salt thereof to the plant, the plant material, or the vicinity of the plant or plant material so as to thereby treat the plant or plant material against pest infestation, wherein the compound of Formula I is applied at an amount such that plant or plant material being treated is exposed to an amount of the compound of Formula I that is higher than the amount the plant or plant material would be exposed to from secretions of Pseudozyma aphidis that (a) exist naturally on the plant or plant material, or in the vicinity thereof or (b) is introduced artificially to the plant, plant material, or vicinity thereof.

The present invention provides a method for controlling and/or preventing of fungal attack on soil, plant, root, foliage, seed, locus of the fungus, or a locus where fungal infestation is to be prevented, wherein the method comprises applying a fungicidally effective amount of at least one compound of Formula I as described herein or an agriculturally acceptable salt thereof to the soil, plant, roots, foliage, seed, locus of the fungus, or locus in which the infestation is to be prevented so as to thereby control and/or prevent fungal attack, wherein the compound of Formula I is produced synthetically.

The present invention provides a method for controlling and/or preventing of fungal attack on soil, plant, root, foliage, seed, locus of the fungus, or a locus where fungal infestation is to be prevented, wherein the method comprises applying a fungicidally effective amount of at least one compound of Formula I as described herein or an agriculturally acceptable salt thereof to the soil, plant, roots, foliage, seed, locus of the fungus, or locus in which the infestation is to be prevented so as to thereby control and/or prevent fungal attack, wherein the compound of Formula I is applied free of fungal material from Pseudozyma aphidis.

The present invention provides a method for controlling and/or preventing of fungal attack on soil, plant, root, foliage, seed, locus of the fungus, or a locus where fungal infestation is to be prevented, wherein the method comprises applying a fungicidally effective amount of at least one compound of Formula I as described herein or an agriculturally acceptable salt thereof to the soil, plant, roots, foliage, seed, locus of the fungus, or locus in which the infestation is to be prevented so as to thereby control and/or prevent fungal attack, wherein the compound of Formula I is applied at an amount such that the soil, plant, root, foliage, seed, locus of the fungus, or locus being treated is exposed to an amount of the compound of Formula I that is higher than the amount the soil, plant, root, foliage, seed, locus of the fungus, or locus would be exposed to from secretions of Pseudozyma aphidis that (a) exist naturally at the soil, plant, root, foliage, seed, locus of the fungus, or locus, or (b) is introduced artificially to the soil, plant, root, foliage, seed, locus of the fungus, or locus.

While the present invention has been shown and described with reference to preferred embodiments thereof, it will be understood by those skilled in the art that many alternatives, modifications and variations may be made thereto without departing from the spirit and scope thereof. Accordingly, it is intended to embrace all such alternatives, modifications, and variations that fall within the spirit and broad scope of the appended claims.

Each embodiment disclosed herein is contemplated as being applicable to each of the other disclosed embodiments. Thus, all combinations of the various elements described herein are within the scope of the invention. In addition, the elements recited in method embodiments can be used in the use, composition, and process embodiments described herein and vice versa.

All publications, patents and patent applications mentioned in this specification are herein incorporated in their entirety by reference into the specification, to the same extent as if each individual publication, patent or patent application was specifically and individually indicated to be incorporated herein by reference.

The following examples illustrate the practice of the present invention in some of its embodiments but should not be construed as limiting the scope of the invention. Other embodiments will be apparent to one skilled in the art from consideration of the specification and examples. It is intended that the specification, including the examples, is considered exemplary only without limiting the scope and spirit of the invention.

### EXAMPLES

### Example 1—Preparation of 2-octyl-2 pentenedioic acid

### Example 1a—Preparation of 2-octyl-2 pentenedioic acid (Crude Extract and Purified Extract from Pseudozyma aphidis)

PDA plates (9 cm diameter) were inoculated by transferring and spreading 50-100 μL of a frozen glycerol stock of P. aphidis L12 with CFU 1×108-1×109 CFU/mL. The PDA plates were then incubated for 48 hours at 25° C. in the dark. The biomass was then harvested from one confluent lawn on PDA and re-suspended into 7.5 mL of sterile 20% glycerol, and the suspension was thoroughly mixed by vortexing. 1 L of sterilized production medium comprising 40 g of glycerol, 1 g of yeast extract, 3% (w/v) of activated amberlite resin and the remainder water, was placed in 2 L Erlenmeyer flasks. The amberlite resin used was an insoluble macroporous polystyrene based resin in the form of small (0.25-0.5 mm radius) microbeads.

The flasks were then inoculated using 2.5 mL of the fresh working P. aphidis L12 cell suspension (as prepared above). The cultures were then incubated on rotary shaker with 5 cm orbit diameter at 150 rpm, 28° C. for 17 days. A fine mesh sieve was used to separate the resin from the culture broth. The resin was then rinsed with water using approximately 2 volumes of water per mass of wet resin by pouring the water directly over the resin on the sieve. The adsorbed material was eluted from the amberlite resin in a volume of methanol, equal to the starting volume of the production medium used for cultivation. 2-octyl-2 pentenedioic acid was isolated and purified from the eluted material using methods known in the art, such as for example flash chromatography and preparative HPLC.

### Example 1b—Preparation of 2-octyl-2 pentenedioic acid (Synthetic)

Synthesis of 2-octyl-2 pentenedioic acid was carried out as follows: diethyl malonate (10 g, 62.5 mmol) was added dropwise to the solution of pentane-washed sodium hydride (1.5 g, 62.5 mmol) in THF (200 mL). The mixture was stirred for 10 min and 1-bromooctane (12.06 g, 62.5 mmol) was added. The resulting mixture was stirred for 2 days. Then, the reaction mixture was quenched with water and extracted with MTBE. The organic extracts were combined, washed with brine, dried over magnesium sulfate, filtered, and evaporated in vacuum. The residue was distillated under reduced pressure to give 14 g of diethyl 2-octylmalonate (51.4 mmol, 82% yield). The diethyl 2-octylmalonate (9.08 g, 33.4 mmol) was added dropwise to the solution of pentane-washed sodium hydride (0.80 g, 33.4 mmol) in tetrahydrofuran (100 mL). The mixture was stirred for 10 min and ethyl-3-chloroacrylate (4.03 g, 33.4 mmol) was added there. The resulting mixture was heated under reflux for 14 h. After cooling to r. t., water was added and the mixture was extracted with Et₂O several times. Combined organic layers were washed with brine, dried over magnesium sulfate, filtered, and evaporated to afford 10.5 g of the crude triethyl-undec-1-ene-1,3,3-tricarboxylate (28.3 mmol, 85% yield) as oil, which was used in the next step without additional purification. triethyl-undec-1-ene-1,3,3-tricarboxylate (10.5 g, 28.4 mmol) was dissolved in ethanol(20 mL) and water (150 mL) containing sodium hydroxide (10 g, 250 mmol) was added. The mixture was refluxed for 8 h, cooled, and adjusted to pH=1 with concentrated hydrochloric acid. Then, the mixture was refluxed for 1 h, cooled, and extracted with Et₂O. The combined organic layers were washed with saturated sodium chloride, dried over magnesium sulfate, filtered, and evaporated under reduced pressure. The crude product was recrystallized from hexane to give 1.2 g of target compound (4.95 mmol, 17.5% yield) as a white crystals.

### Example 1c—Preparation of 2-octyl-2 pentenedioic acid (Synthetic)

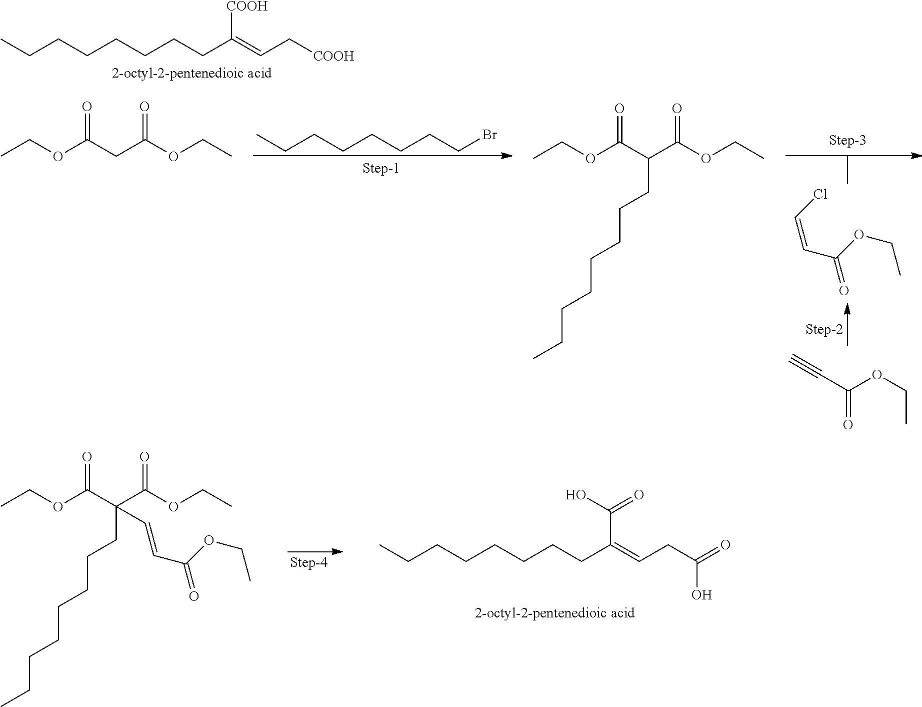

### Step 1: synthesis of diethyl-2-octylmalonate

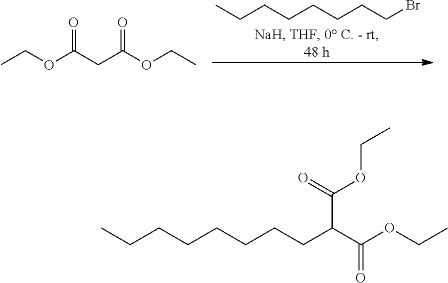

Procedure 1: With Sodium Hydride as Base

Procedure: To a stirred suspension of sodium hydride (60%, 29.96 g, 749.2 mmol) in THF (1.5 L), diethyl malonate (100 g, 624.3 mmol) was added drop wise over a period of 45 min at 0° C. under nitrogen atmosphere. The above suspension was stirred for another 15 min at the same temperature. Octyl bromide (96.45 g, 499.4 mmol) was added drop wise to the above medium and the resultant reaction mixture was allowed to bring to room temperature and stirred for another 48 h at the same temperature.

The progress of the reaction was monitored by GC, and observed about 45% of the required product peak apart from both unreacted starting materials.

Work up: The reaction mixture was quenched with ice cold water (7.5 L), extracted to ethyl acetate (2×1 L), and the combined organic layer was dried over anhydrous sodium sulphate, filtered and concentrated under reduced pressure to get the crude product as pale-yellow liquid.

Purification: The crude compound was purified by high vacuum distillation (0.3 mm of Hg) and the required product was collected at 171° C. to 175° C. oil bath temperature at 122° C. to 125° C. vapor temperature as colorless liquid [Yield: 25 g (59%), Purity by GC: 97.67%].

Procedure 2: With Sodium Ethoxide as Base

Procedure: To a stirred suspension of sodium (1.43 g, 62.4 mmol) in ethanol (80 mL), diethyl malonate (10 g, 62.4 mmol) was added drop wise over a period of 15 min at room temperature under nitrogen atmosphere. The above suspension was stirred for another 15 min at same temperature. Bromo octane (10.2 g, 53 mmol) was added drop wise, to the above medium and the resultant reaction mixture was allowed to bring to room temperature and stirred for another 18h at 30° C. under nitrogen atmosphere.

The progress of the reaction was monitored by GC, and observed about 64.78% of the required product apart from both starting materials and along with minor impurities.

Work up: Ethanol was removed under reduced pressure, and the residue was diluted with methyl tert-Butyl ether (MTBE, 100 mL) and washed with hydrochloric acid (1N HCl, 2×30 mL). The MTBE layer was washed with water, dried over anhydrous sodium sulphate, filtered and concentrated under reduced pressure to get the crude material. The GC analysis showed about 73.28% of the required product apart from both starting materials and along with minor impurities.

Purification: The crude compound was purified by high vacuum distillation (0.2 mm Hg) and the required product was collected at 163 to 168° C. oil bath temperature at 120 to 123° C. vapor temperature as colorless liquid [Yield: 11.5 g (68%), Purity by GC: 94.10%].

### Step 2: synthesis of ethyl-3-chloroacrylate

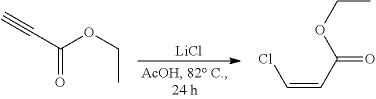

Procedure: To a stirred suspension of ethyl propiolate (50 g, 509.6 mmol) and lithium chloride (32.41 g, 764.5 mmol) in acetonitrile (500 mL), acetic acid (131.17 mL) was added drop wise over a period of 1 h at 50° C. under nitrogen atmosphere. The above reaction medium was heated to 82° C. for 14 h. The progress of the reaction was monitored by GC, observed about 79% of the required product and 20% of the starting material was left out. The reaction medium was treated with additional 0.6 eq. of lithium chloride and 1 eq. of acetic acid and the resultant reaction medium were stirred for another 6 h. The progress of the reaction was monitored by GC, and observed about 83.34% of the required product apart from ethyl propiolate. Based on the analytical data, added additionally another 0.3 eq. of lithium chloride and heating continued further 4 h. The progress of the reaction was monitored by GC, and observed about 98.73% of the required product apart from ethyl propiolate (1.27%).

Work up: Acetonitrile was removed under reduced pressure and the residue obtained was suspended in ice cold water and extracted to ethyl acetate. The organic layer was dried over anhydrous sodium sulphate, filtered and concentrated under reduced pressure to get the crude product as pale-yellow liquid [Yield: 61.5 g (90%), Purity by GC: 98.13%].

### Step 3: Synthesis of triethyl undec-1-ene-1, 3, 3-tricarboxylate

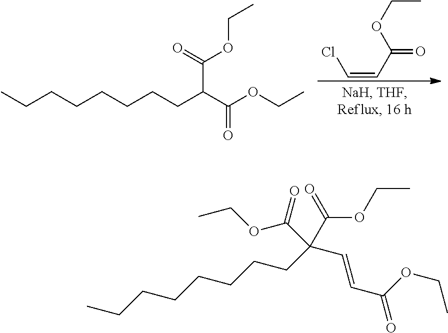

Procedure: To a stirred suspension of sodium hydride (60%, 3.52 g, 88.2 mmol) in THF (225 mL), diethyl-2-octylmalonate (25 g, 55.1 mmol) was added drop wise over a period of 40 min under nitrogen atmosphere at 0° C. The above suspension was stirred for another 15 min at the same temperature. Ethyl-3-chloro acrylate (11.08 g, 82.7 mmol) was added drop wise to the above medium and the resultant reaction mixture was allowed to bring to room temperature and heated to reflux for 16 h. The progress of the reaction was monitored by GC, and observed about 78.04% of the required product peak along with a major impurity (10%) at 21.51 RT. Both the starting materials were consumed based on this stoichiometric ratio of reagents.

Work up: The reaction mixture was quenched with ice cold water (3 L), extracted to ethyl acetate (2×150 mL), and the combined organic layer was dried over anhydrous sodium sulphate, filtered and concentrated under reduced pressure to get the crude product as pale-yellow liquid.

Purification: The crude compound was purified by high vacuum distillation (at 0.3 mm Hg) and the required product was collected at 185° C. to 200° C. oil bath temperature at 163° C. to 170° C. vapor temperature as pale-yellow color liquid [Yield: 22 g (65%), Purity by GC: 94.21%].

### Step 4: Synthesis of 2-octyl-2-pentenedioic acid

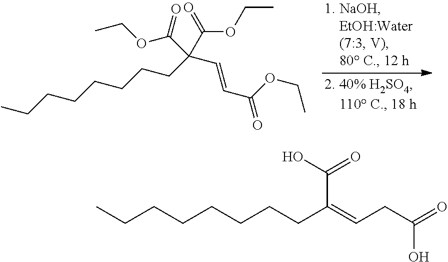

Part a:

Procedure: To a solution of (10 g, 27.02 mmol) in ethanol: water (70 mL: 30 mL), sodium hydroxide (4.8 g, 121.6 mmol) was charged and the resultant reaction medium was heated to 80° C. for 12 h. The progress of the reaction was monitored by HPLC analysis, and observed about 64% conversion of the required product along with remaining 31% as the intermediate product in which the double bond was not migrated.

Work up: The reaction mixture was diluted with water (150 mL) and extracted with ethyl acetate (2×75 mL), to get rid of any unreacted materials. The aqueous layer was acidified to pH=1 with concentrated hydrochloric acid (22 mL)under ice cold condition and stirred for 15 min. The precipitated solid was filtered off, washed with water and dried under vacuum to get the crude product as off-white solid [Yield: 5.8 g (88%), Purity by GC: 64%: 31%].

Part b:

Procedure: The suspension of the isolated crude solid of(5.8 g), in 40% sulphuric acid (58 mL) was heated to 110° C. for 18 h. The resulted ethanol was removed via distillation from the reaction mixture simultaneously through a distillation set up. The reaction progress was monitored by HPLC analysis and observed about 71% of the required product along with some other minor impurities.

Work up: The reaction mixture was diluted with water (90 mL) and extracted with ethyl acetate (2×50 mL). The combined organic layer was dried over anhydrous sodium sulphate, filtered, and evaporated under reduced pressure to get the crude product as off-white solid. This crude material was further triturated with hexanes (60 mL) and the medium was stirred at room temperature for 20 min. The precipitated solid was filtered, washed with hexanes and dried under high vacuum to get the product as off-white solid [Yield: 4.2 g (65%), Purity by HPLC: 97.23%]. Also, the final compound was further characterized by 1H NMR & 13C NMR.

### Example 2—Evaluation of Fungicidal Activity

### Example 2a—Efficacy of 2-octyl-2 pentenedioic acid against B. cinerea

2-octyl-2 pentenedioic acid was also evaluated for antifungal activity using bio-assay in liquid medium using B. cinerea as an indicator strain. The bio-assay was carried out in PDB liquid medium.

The bio-assay for determination of antifungal activity was set-up based on the procedure as described in Stammler and Speakman, 2006. Microtiter Method to Test the Sensitivity of Botrytis cinerea to Boscalid. Journal of Phytopathology, 154, 7-8, p 508-510. This bio-assay is based on dilution of the antimicrobial agent and determination of growth inhibition of a sensitive indicator strain, in this case Botrytis cinerea. The bio-assay was performed in 96-well microtiter plates and included micro-dilutions of the antimicrobial agent in a liquid growth medium, where each well was inoculated with a standardized number of fungal spores of the indicator strain. After incubation (no agitation, 18° C. in darkness for 5 days), the results were obtained by measurement of fungal growth by photometer at 405 nm and the values were corrected by comparison with the blanks (cultures without the antimicrobial compound). The ED (effective dose) values are compared with reference standard fungicides.

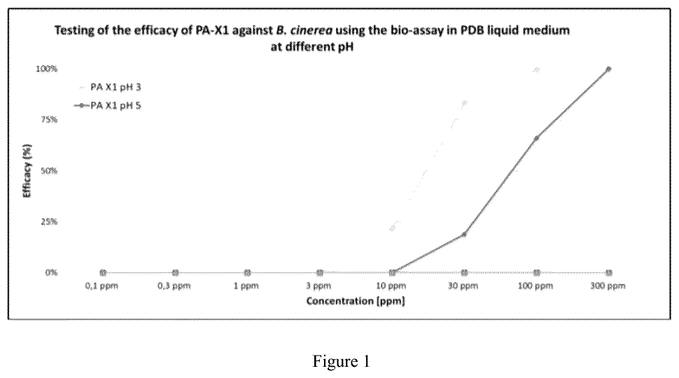

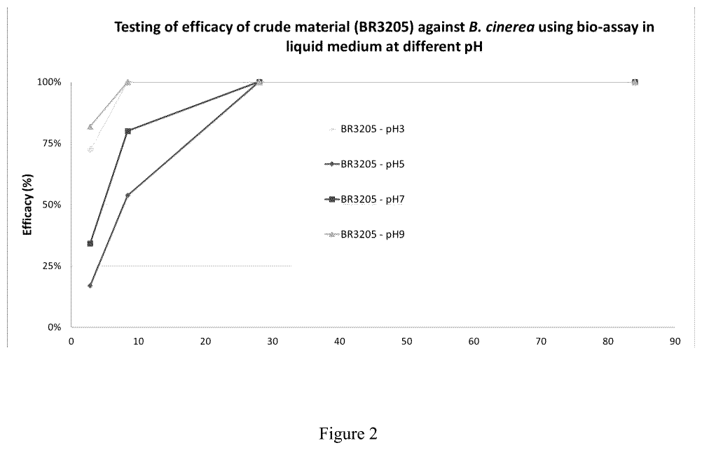

The results are shown in FIGS. 1 and 2.

In particular, as can be seen in FIG. 1, 2-octyl-2 pentenedioic acid (PA X1) provides excellent efficacy against B. cinerea at both a pH of 3 as well as a pH of 5.

As can be seen in FIG. 2, the Pseudozyma aphidis crude extract (BR3205), which comprises 2-octyl-2 pentenedioic acid, provides excellent efficacy against B. cinerea.

In FIGS. 1 and 2, the 2-octyl-2 pentenedioic acid was purified from the crude extract (not synthetic).

### Example 2b—Efficacy of 2-octyl-2 pentenedioic acid against P. infestans

Table 1 demonstrates the in vitro efficacy (ED50 and ED90 μg DW/ml or μg a.i.iml or ppm) of (i) the crude extract extracted from P. aphidis, (ii) the purified extract 2-octyl-2 pentenedioic acid and (iii) the synthetic product 2-octyl-2 pentenedioic acid towards P. infestans. The P. infestans was cultivated on V8 juice liquid media and was adjusted to pH 4.6, 6.2 or 8.0.

As can be seen from the data provided in Table 1, 2-octyl-2 pentenedioic acid has activity against P. infestans, especially at a pH of 4.6 and 6.2. Further, it can be noted that the crude extract, which comprises 2-octyl-2 pentenedioic acid, has activity against P. infestans (from other components found in the crude extract).

TABLE 1
In vitro evaluation of P. aphidis crude extract, purified
extract of 2-octyl-2 pentenedioic acid and synthetic
2-octyl-2 pentenedioic acid towards P. infestans

| | | | 2-octyl-2 | 2-octyl-2 |
|---|---|---|---|---|
| | | | pentenedioic | pentenedioic |
| | | Crude | acid (from | acid |
| | | extract | extract) | (synthetic) |
| Efficacy | pH | (ppm) | (ppm) | (ppm) |
| ED₅₀ | 4.6 | 37 | 10 | 19 |
| | 6.2 | 155 | 17 | 22 |
| | 8.0 | 175 | Not detected | Not detected |
| ED₉₀ | 4.6 | 60 | 37 | 41 |
| | 6.2 | 230 | 41 | 42 |
| | 8.0 | 415 | Not detected | Not detected |

### Example 2c—Efficacy of 2-octyl-2 pentenedioic acid against Z. tritici

Table 2 demonstrates the in vitro efficacy (ED50 and ED90 μg DW/ml or μg a.i./ml) of the crude extract extracted from P. aphidis towards Z. tritici. The Z. tritici was cultivated on V8 juice liquid media and was adjusted to pH 4.6, 6.2 or 8.0. As can be seen from the data provided in Table 2, the crude extract, which includes 2-octyl-2 pentenedioic acid has activity against Z. tritici.

TABLE 2
In vitro evaluation of P. aphidis crude extract towards Z. tritici

| | | Crude |
|---|---|---|
| | | extract |
| Efficacy | pH | (ppm) |
| ED₅₀ | 4.6 | 70 |
| | 6.2 | 300 |
| | 8.0 | 330 |
| ED₉₀ | 4.6 | 120 |
| | 6.2 | 450 |
| | 8.0 | 470 |

### Example 2d—Efficacy of 2-octyl-2 pentenedioic acid against Puccinia triticina

This experiment illustrates the effect of the crude extract (YE) of P. aphidis, synthetic (E)-2-octyl-2-pentenedioic acid (PA), extracted 2-octyl-2 pentenedioic acid from Pseudozyma aphidis in methanol, Rhapsody® (Bacillus subtilis) and Amistart (azoxystrobin) at 0.1% on the intensity of infection of wheat leaf fragments by uredospores of Puccinia triticina strain BT06M215. Rhapsody® (Bacillus subtilis) and Amistar® (azoxystrobin) are commercially available fungicidal solutions. P. titicina strain BT06M215 was isolated from untreated wheat leaves in France.

All fungicidal products were prepared in a volume of water corresponding to 200 L/ha.

Winter wheat seedlings (cv. ALIXAN. LG Seeds) at the second leaf stage (BBCH 12) were treated by:
Crude extract at 280 mg dry weight/ml, tested at 0.25%-0.5% and 1.0% in 0.05% Tween® 80
PA, (E)-2-octyl-2-pentenedioic acid, prepared in methanol at 280 mg a.i./ml, tested at 0.25%-0.5% and 1.0% in 0.05% Tween® 80
Rhapsody®, SC of Bacillus subtilis strain QST 713 at 109 cfu/g, tested at 2 L/ha and prepared in a volume of 0.05% Tween® 80
Amistar®, SC of Azoxystrobin at 250 g a.i./L, tested at 1 L/ha
Control 1: 0.05% Tween® 80
Control 2: 0.05% Tween® 80+1.0% Methanol

For each experimental condition tested, 18 replicates of one wheat leaf were tested. Treated wheat seedlings were placed in a climatic chamber permitting disease development. Disease assessment (intensity of infection) on the first leaf of each wheat plantlets is monitored at 8 to 15 days post inoculation.

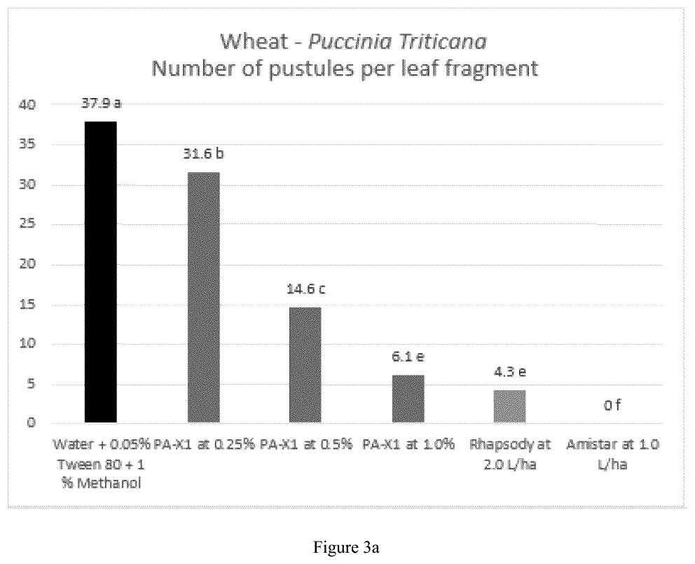

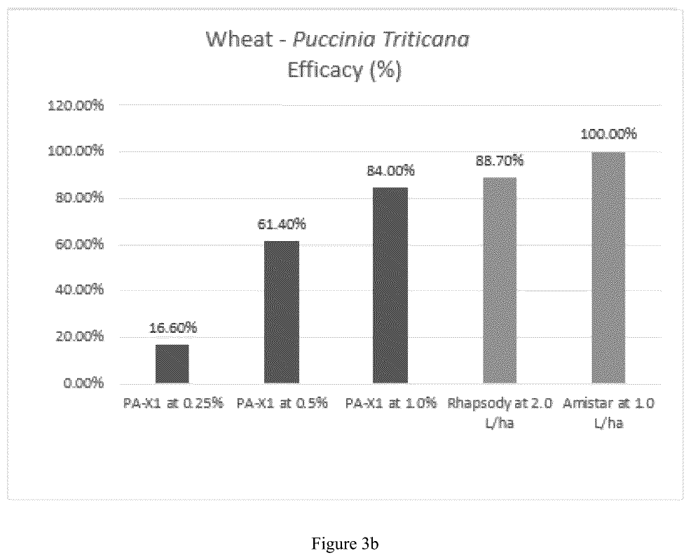

As can be seen in FIGS. 3a and 3b, the synthetic (E)-2-octyl-2-pentenedioic acid (PA-X1) has comparable efficacy against Puccinia triticina as Rhapsody® (applied at 2.0 L/ha) which is a fungicidal solution currently in the market.

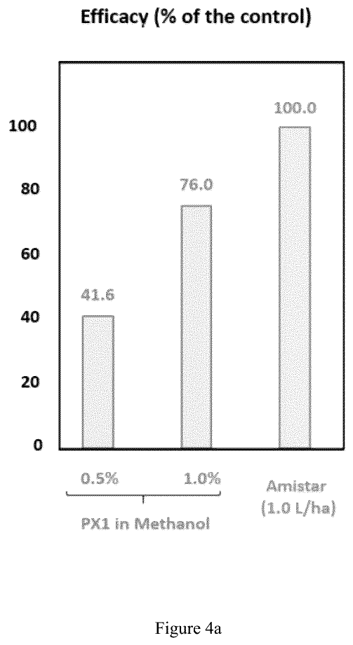

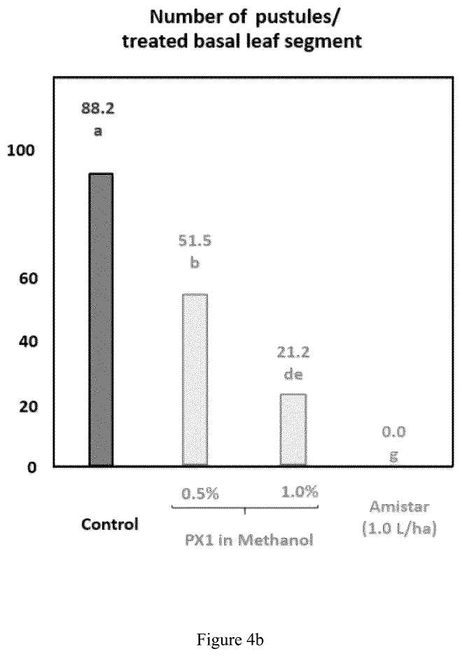

As can be seen in FIGS. 4a and 4b, extracted 2-octyl-2 pentenedioic acid in methanol applied at 100 g/ha showed comparable efficacy against Puccinia triticina as Amistar® (applied at 1 L/ha) which is a fungicidal solution currently in the market.

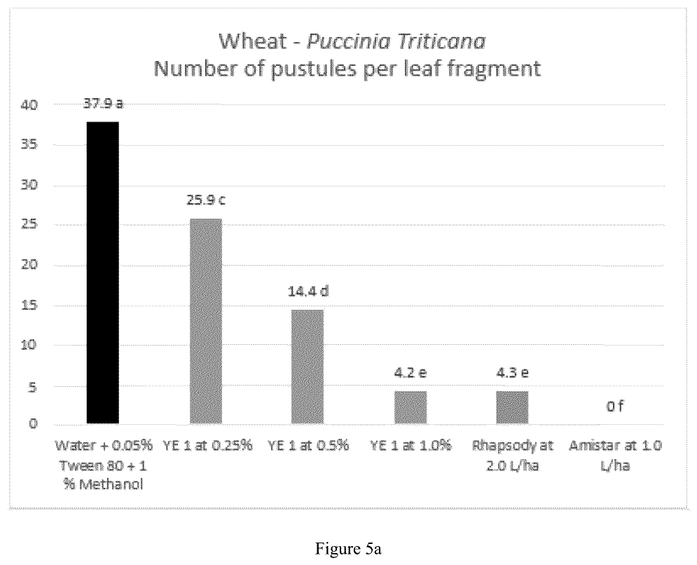

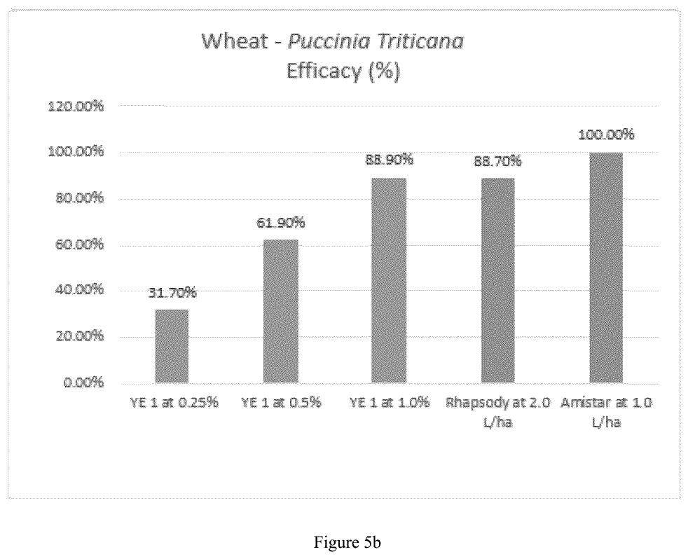

As can be seen in FIGS. 5a and 5b, the crude extract of P. aphidis (YE) applied at 1% showed comparable efficacy against Puccinia triticina as Rhapsody® (applied at 2.0 L/ha) which is a fungicidal solution currently in the market.

In particular, YE at 0.25% demonstrates 35% efficacy. The spraying solution is of 200 L/ha which corresponds to an application rate of PA-X1 of 1.4 g/ha. Similarly, YE at 0.5% demonstrated over 60% efficacy. The spaying solution is of 200 L/ha which corresponds to an application rate of PA-X1 of 2.8 g/ha. Further, YE at 1% demonstrated about 90% efficacy. The spaying solution is of 200 L/ha which corresponds to an application rate of PA-X1 of 5.6 g/ha.

### Example 2e—Efficacy of 2-octyl-2 pentenedioic acid Against Zymoseptoria tritici Compared to Amylo-x® and Proline®

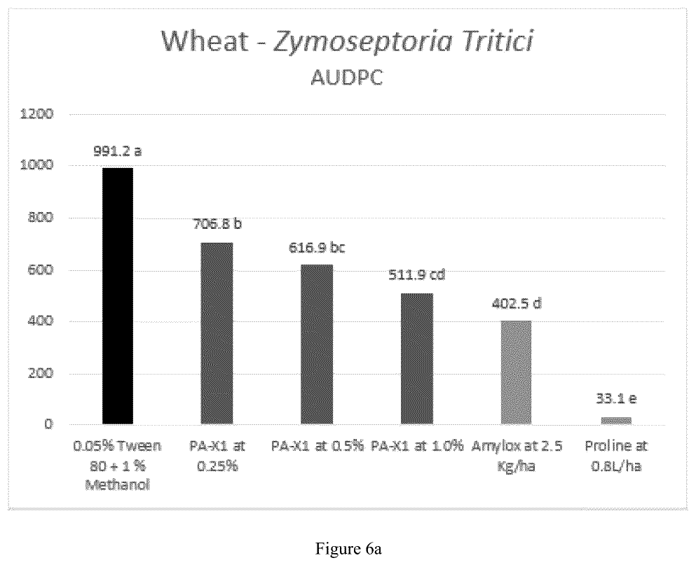

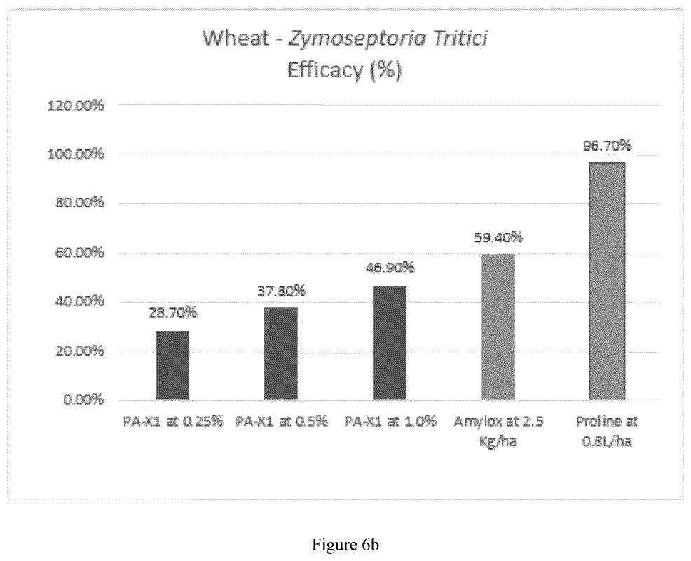

FIGS. 6a and 6b illustrates the effect of the synthetic (E)-2-octyl-2-pentenedioic acid (PA-X1), and Amylo-x® (Bacillus amyloliquefaciens) and Proline® (prothioconazole) on the intensity of infection on wheat leaf fragments cv. ALIXAN by Zymoseptoria tritici strain Mg Tri-R6. Amylo-x® (Bacillus amyloliquefasciens) and Proline® (prothioconazole) are commercially available fungicidal solutions.

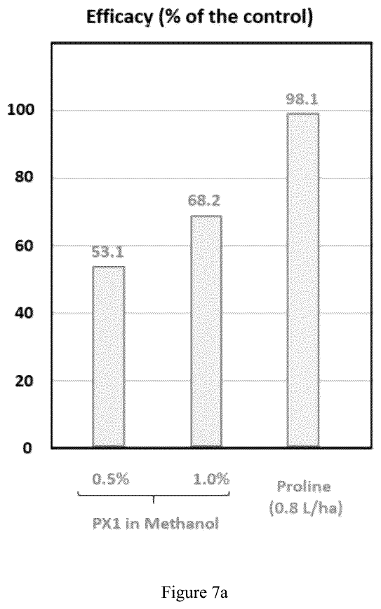

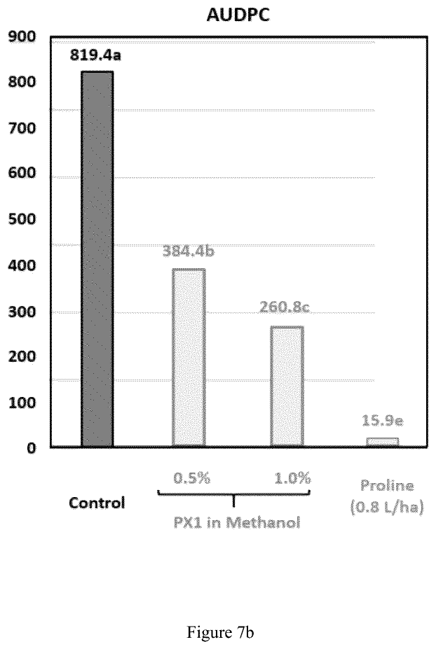

As can be seen in FIGS. 7a and 7b, extracted 2-octyl-2 pentenedioic acid from P. aphidis in methanol (PX1—in Methanol) (applied at 100 gr/ha) showed comparable efficacy against Zymoseptoria tritici strain Mg Tri-R6 as Proline® (applied at 0.8 L/ha) which is a fungicidal solution currently in the market.

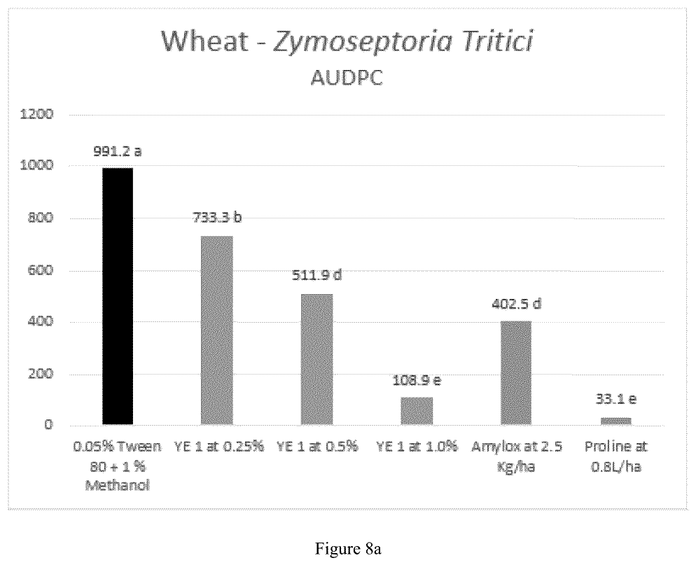

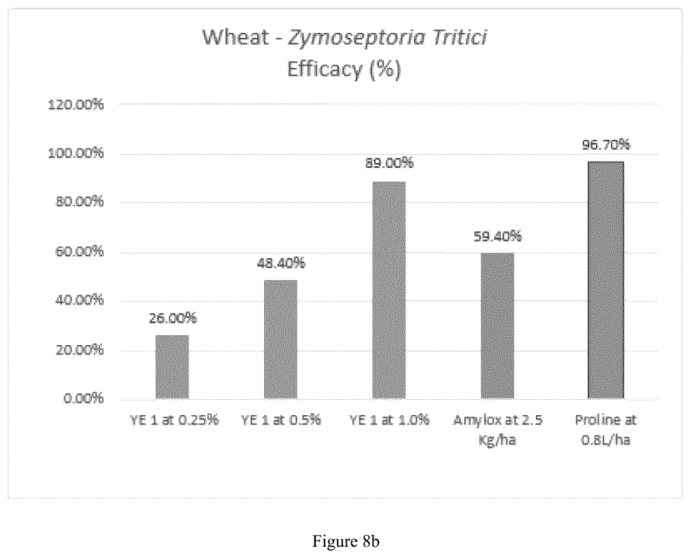

FIGS. 8a and 8b illustrate the effect of the crude extract (YE) of P. aphidis at 1%, Amylo-x® (Bacillus amyloliquefasciens) and Proline® (prothioconazole) on the intensity of infection on wheat leaf fragments cv. ALIXAN by Zymoseptoria tritici strain Mg Tri-R6. Amylo-x® (Bacillus amyloliquefaciens) and Proline® (prothioconazole) are commercially available fungicidal solutions.

Z. tritici strain was isolated from untreated wheat leaves in 2008 in France. The isolate is moderately resistant to DMI fungicides (TriMR) and highly resistant to QoI fungicides (G143A mutation).

All fungicidal products were prepared in a volume of water corresponding to 200 L/ha.

Winter wheat seedlings (cv. ALIXAN. LG Seeds) at the second leaf stage (BBCH 12) were treated by:
crude extract at 280 mg dry weight/ml, tested at 0.25%-0.5% and 1.0%, all prepared in 0.05% Tween® 80.
PA, (E)-2-octyl-2-pentenedioic acid, prepared in methanol at 280 mg a.i./ml, tested at 0.25%-0.5% and 1.0%, all prepared in 0.05% Tween®80.
Amylox®, SC of Bacillus amyloliquefaciens subsp. plantarum strain D74 at 5.1010 cfu/g, tested at 2.5 Kg/ha and prepared in 0.05% Tween® 80
Proline®, EC of Prothioconazole at 250 g a.i./L, tested at 0.8 L/ha
Control 1: 0.05% Tween® 80
Control 2: 0.05% Tween® 80+1.0% of Methanol

For each experimental condition tested, 18 replicates of one wheat leaf were tested. Treated wheat seedlings were placed in a climatic chamber optimizing disease development. Disease assessment (Intensity of infection) on the first leaf of each wheat plantlets is monitored at 21 and 28 days post inoculation.

### Example 2f—Efficacy of 2-octyl-2 pentenedioic acid Against Phytophthora infestans Compared to LBG 01F34® and Revus®

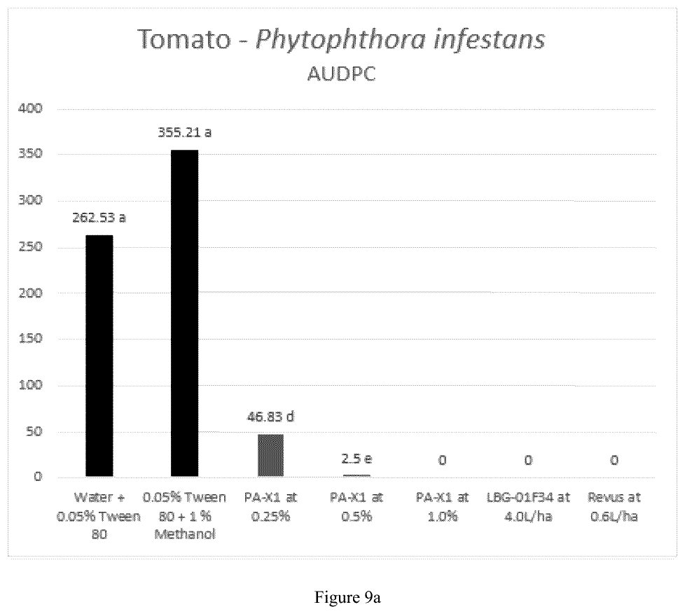

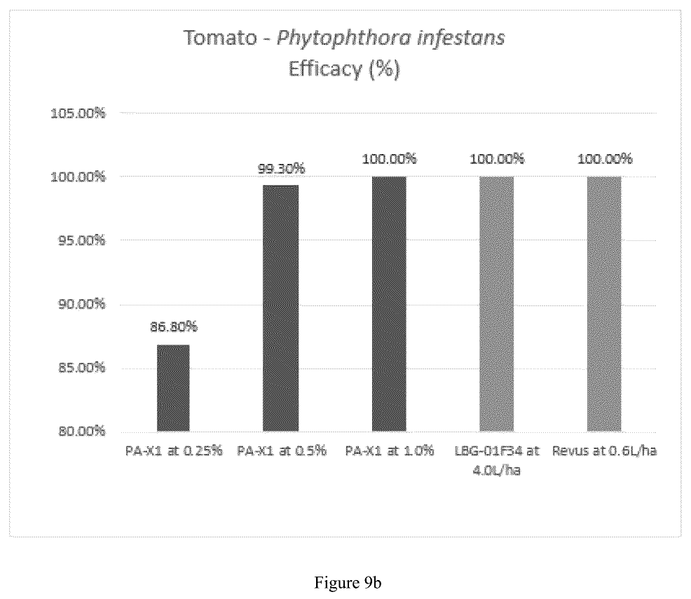

FIGS. 9a and 9b illustrate the effect of the synthetic (E)-2-octyl-2-pentenedioic acid (PA-X1), LBG 01F34® (potassium phosphonates) and Revus® (mandipropamid) on the intensity of infection on tomato plants var. microtome by Phytophthora infestans strain Pi61. LBG 01F34® (potassium phosphonates) and Revus® (mandipropamid) are commercially available fungicidal solutions.

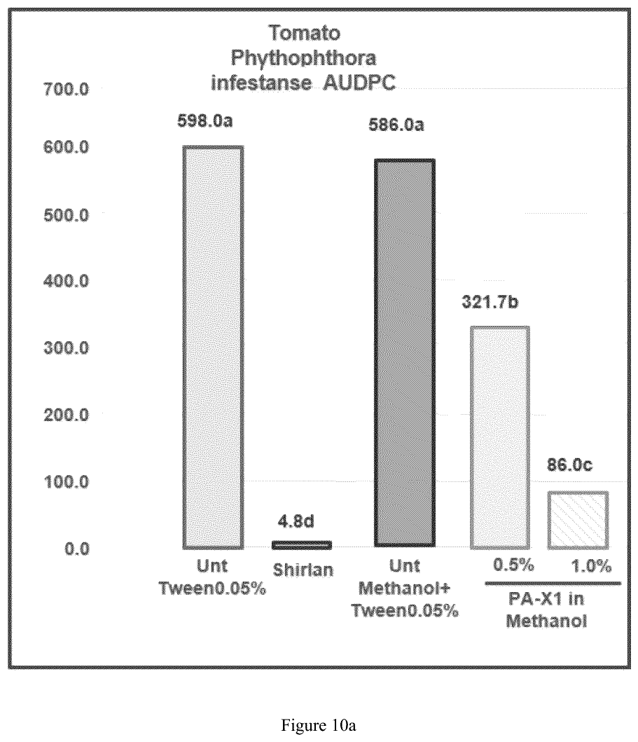

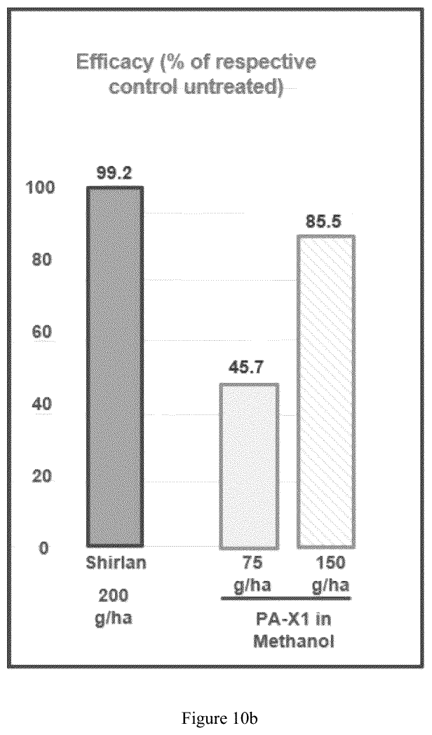

FIGS. 10a and 10b illustrate that the synthetic 2-octyl-2 pentenedioic acid (PA-X1) in methanol (applied at 150 g/ha) has comparable efficacy against Phytophthora infestans as Shirlan (fluazinam) (applied at 200 g/ha) which is a fungicidal solution currently in the market.

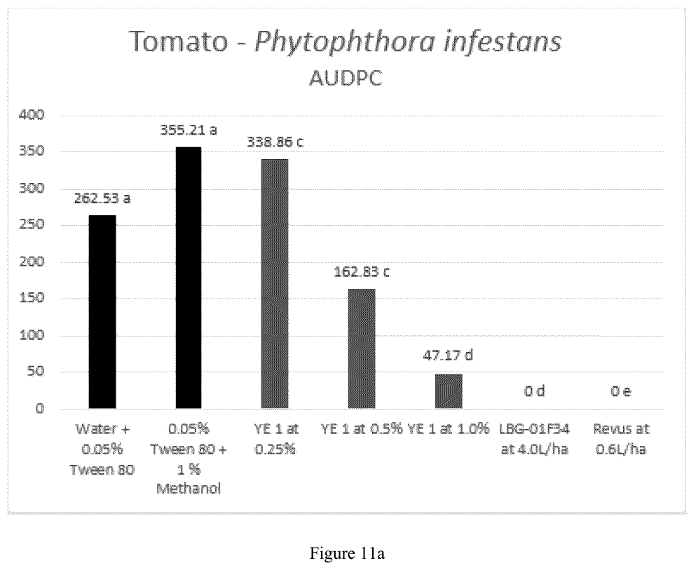

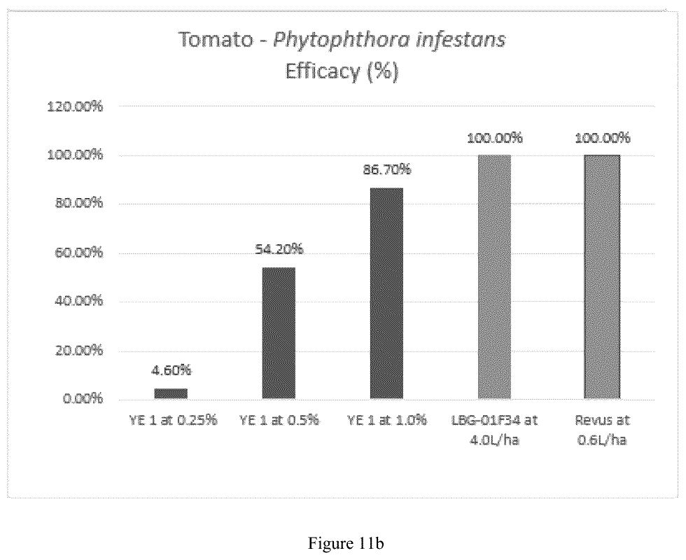

FIGS. 11a and 11b illustrate the effect of the crude extract (YE) of P. aphidis, LBG 01F34® (potassium phosphonates) and Revus® (mandipropamid) on the intensity of infection on tomato plants var. microtome by Phytophthora infestans strain Pi61. LBG 01F34® (potassium phosphonates) and Revus® (mandipropamid) are commercially available fungicidal solutions.

Material and Methods for Phytophthora infestans

P. infestans PI61 was isolated from untreated tomato leaves in 2017 originated from Denmark. The isolate is susceptible towards all fungicides.

All fungicidal products were prepared in a volume of water corresponding to 300 L/ha.

Tomato plantlets (ev. Marmande) at the third leaf stage were treated by:
crude extract at 280 mg dry weight/ml, tested at 0.25%-0.5% and 1.0% in 0.05% Tween® 80
PA, (E)-2-octyl-2-pentenedioic acid, prepared in methanol at 280 mg a.i./ml, tested at 0.25%-0.5% and 1.0% in 0.05% Tween® 80
Potassium phosphonates based fungicide named: LBG-01F34®, tested at 4 L/ha
Revus® SC at 250 g a.i./L, tested at 0.6 L/ha
Control 1: 0.05% Tween® 80
Control 2: 0.05% Tween® 80+1.0% of Methanol

For each experimental condition tested, 15 replicates of one tomato leaflet were tested. Treated plantlets were placed in a climatic chamber permitting disease development. Disease assessment (Intensity of infection) of each leaflet is monitored between 4 to 12 days post inoculation.

### Example 3: Advantage of Using Nitrogen-Limiting Conditions

### Example 3a. Activity of P. aphidis Extracts/Eluates Against B. cinerea Using the Bioassay a in Liquid Medium

The results are shown in FIG. 12. As can be seen in FIG. 12, pure methanol was toxic at concentrations above 3%, reaching approximate ED50 at maximal 10% (v/v). Highest activity (ED50=approx. 1-2% v/v) was observed from a with highly limited nitrogen source (sample 2, 40 g/L glucose+1 g/L yeast extract; C:N=40:1, cultivated for 10 days). Negative control was resin eluate from PDB without P. aphidis, which showed same activity as pure methanol.

### Example 3b. Activity of P. aphidis Extracts with Amberlite Resin Against B. cinerea Using an Agar Plates Assay

General Description:

A liquid bio-assay for determination of antifungal activity was set-up using a published procedure, using Botrytis cinerea as indicator strain (Stammler and Speakman, 2006; DOI: 10.1111/j.1439-0434.2006.01139.x).

This bio-assay is a microbial susceptibility testing method and it is based on dilution of the antimicrobial agent and determination of growth inhibition of a sensitive indicator strain. The bio-assay is performed in 96-well microtiter plates and includes micro-dilutions of the antimicrobial agent in a liquid growth medium, where each well is inoculated with a standardized number of fungal spores of the indicator strain (in our case Botrytis cinerea). After incubation (no agitation, 18° C. in darkness for 5 days), the results are obtained by measurement of fungal growth by photometer at 405 nm and the values are corrected by comparison with the blanks (cultures without the antimicrobial compound). The ED (effective dose) values are compared with reference standard fungicides. In our case we initially used three fungicide standards, namely trifloxystrobin, iprodione and pyraclostrobin and additionally, boscalid was also introduced after consultation with Adama. Initially the bio-assay was set-up using the reference fungicide standards.

Procedure Overview (96-Well Flat-Bottom Microwell Plates were Used):
1. Pure fungicides were dissolved in methanol and the dilutions were prepared in sterile water; For P. aphidis samples, 100× concentrated methanol extracts/eluates were diluted in water.
2. 50 μL of fungicide/sample solution was mixed with 50 μL B. cinerea spore suspension in YBA medium to reach final spore count 1×104 spores in a final volume 100 μL per well.
3. The following final concentrations of fungicides were used: 0, 0.01, 0.03, 0.1, 0.3, 1.3, and 10 ppm.
4. For P. aphidis extracts/eluates, 100× concentrated samples were diluted 10×, 30×, 100×, 300×, 1000×, 3000× and 10000× (example: for 10× diluted sample, 10 μL of concentrated sample in methanol was added to the well to a total volume of 100 μL; for 100× diluted sample, 10 μL of 10× diluted sample in water was added to the well to a total volume of 100 μL etc.).
5. For each fungicide/sample, three replicate wells were used.
6. As blanks, fungicides/samples with the same concentration were used in YBA medium without B. cinerea (also in three replicates per blank).
7. The microtiter plates were incubated without agitation in a sealed box (to prevent evaporation) at 18° C. in the dark for 5 days.
8. Growth of B. cinerea was measured using a microplate reader at 405 nm.
9. The values were corrected by comparison with the blanks.

The Procedure for Bioassay (Disk or Droplet Diffusion Assay) on Agar Plates:

The diffusion disk assay was performed using B. cinerea as indicator strain. B. cinerea spores (10\hat{ }5 per mL of media) were added to sterile PDA medium cooled to 55° C. and the agar medium was poured in Petri dishes with 9 cm diameter. 20 μL of sample extract was applied directly to the center of the agar plate as a droplet or on a paper disk, which was then placed on the center of the agar plate. The plates were then incubated at 18 C in the dark for 5-10 days and inhibition zones were visually inspected.

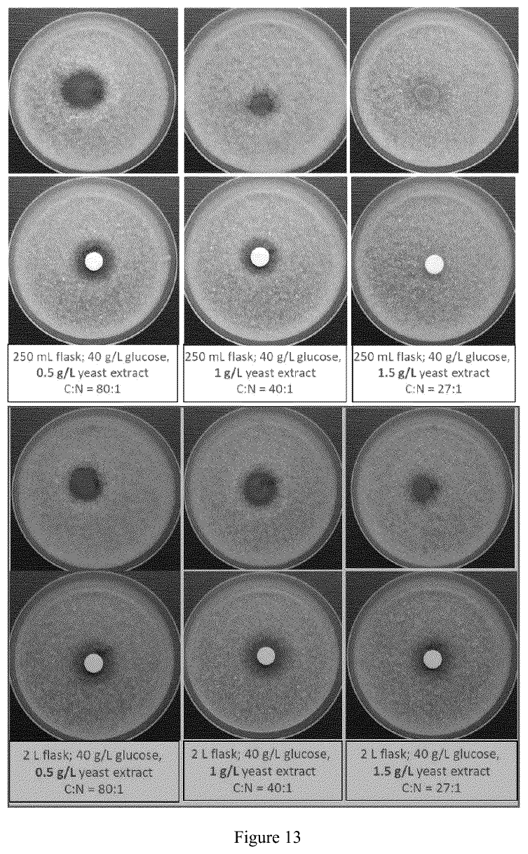

The results are shown in FIG. 13. As can be seen in FIG. 13, there is a clear correlation between inhibition zone size and nitrogen limiting could be observed. In media which cause nitrogen stress P. aphidis produces more antifungal compound(s). The inhibition zones are stable (over 2 weeks).

### Example 4: Improved Extraction Method

Table 3 shows a comparison between various extraction methods of P. aphidis for activity against B. cinerea using the bio-assay with tomato leaves.

TABLE 3

| | | Leaf | | Infection | Infection | Infection |
|---|---|---|---|---|---|---|
| Sample # | Sample | damage | Infection | rate 48 h | rate 72 h | rate 96 h |
| 1 | NO, 70% EtOH | no | NO | − | − | − |
| 2 | NO, 70% EtOH | | YES | + | ++ | +++ |
| 3 | 1 [symbol] 5 g/L Acetic acid in 70% EtOH | | droplet of | − | + | + |
| 4 | 1% Adama CE125 ethyl acetate | | B. cinerea | − | + | ++ |
| | extract in 70% EtOH | | spores (approx. 1000) | | | |
| 5 | 1% Acies CE EtOAc extract from | | spores per leaf) | − | + | + |
| | 150 L fermentation (PAPM3) [symbol] SOP | | | | | |
| 6 | 1% [symbol] 101 resin methanol eluate | | | + | +++ | +++ |
| | from PD [symbol] without P. aphidis | | | | | |
| | (negative control) | | | | | |
| 7 | 1% whole broth methanol extract of | | | − | + | ++ |
| | P. aphidis culture in PDB (10 days) | | | | | |
| 8 | 1% [symbol] 101 resin methanol eluate from | | | − | − | + |
| | P. aphidis culture in PDB (10 days) | | | | | |
| 9 | 1% whole broth methanol extract of | | | − | + | ++ |
| | P. aphidis culture in medium | | | | | |
| | with 40 g/L glycerol and 2 g/L soy flour (10 days) | | | | | |
| 10 | 1% [symbol] 101 resin eluate from P. aphidis | | | − | − | + |
| | culture in medium with 40 g/L glycerol and | | | | | |
| | 2 g/L soy flour (10 days) | | | | | |

[symbol]

indicates data missing or illegible when filed

The untreated control (#2, only 70% ethanol) was taken as a reference for the maximal infection rate. Acetic acid (#3) inhibited B. cinerea infection (fungal mycelium was observed growing on the surface of the leaves, but not in the leaf tissue).

Adama CE (#4) and 1% Acies CE (#5) in ethyl acetate inhibited B. cinerea infection and no mycelium was growing on the surface of the leaves.

Eluate from resin incubated in sterile medium without P. aphidis (#6) showed no effect on the spread of B. cinerea infection, which was highly comparable to the untreated control (#2).

Whole broth methanol extracts from PDB medium (#7) and medium with 40 g/L glycerol and 2 g/L soy flour (#9) significantly reduced the spread of the B. cinerea infection, however the “protective” effect was most significant with methanol eluates obtained from P. aphidis cultures grown in PDB (#8) and medium with 40 g/L glycerol and 2 g/L soy flour (#10) with 3% D101 amberlite resin, where the spread of the infection was completely or significantly inhibited while the leaves showed no tissue damage (wilt).

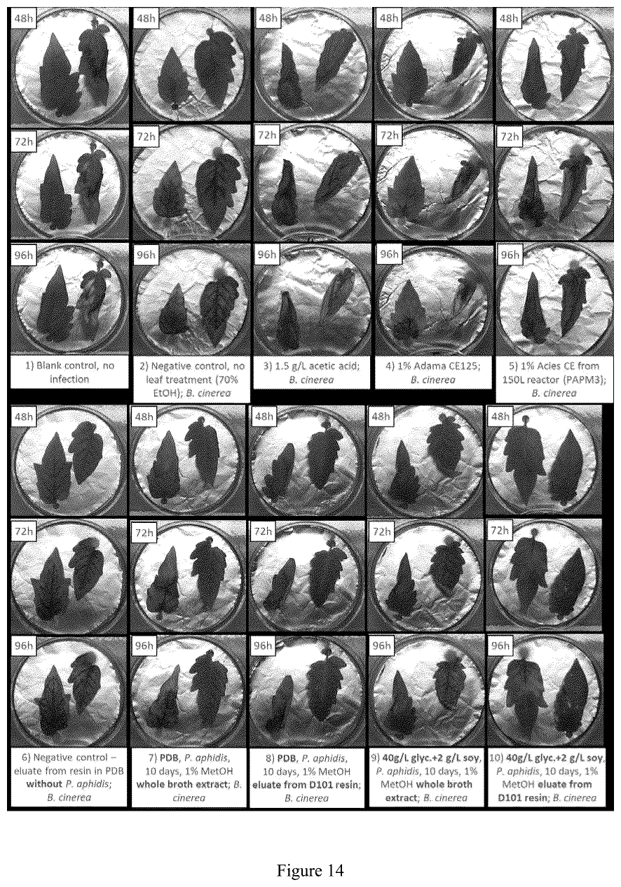

The results are also shown in FIG. 14.

1-120. (canceled)

121. A method of treating a locus against pest infestation comprising applying an effective amount of at least one compound of Formula I: 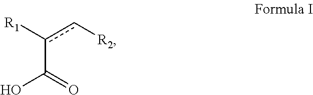
wherein
R₁ is a C₁-C₁₂ alkyl optionally substituted with one or more substituents independently selected from the group consisting of halogen, C(O)R, CN, NH₂, —NH(C₁-C₄ alkyl), —(C₁-C₄ alkyl)—NH₂, —N(C₂-C₄ alkyl)(C₂-C₄ alkyl), OR, —S(O)₂ (C₁-C₄ alkyl), —S(O)(C₁-C₄ alkyl), C(O)OH and C(O)OR,
R₂ is —(C₁-C₁₂)—COOH, —(C₁-C₁₂)—COO, —(C₁-C₁₂)—COOR, —(C₁-C₁₂)—CONH₂, —(C₁-C₁₂)—CONHR, —(C₁-C₁₂)—CONR₂, —(C₁-C₁₂)—COSR, —(C₁-C₁₂)—COX, —(C₁-C₁₂)—S(O)₂NH₂, —(C₁-C₁₂)—S(O)₂NHR, —(C₁-C₁₂)—S(O)₂NR₂, —(C₁-C₁₂)—CN, —(C₁-C₁₂)—CR═NR, —(C₁-C₁₂)—CONHNH₂, wherein each of the (C₁-C₁₂)—COOH, —(C₁-C₁₂)—COO, —(C₁-C₁₂)—COOR, —(C₁-C₁₂)—CONH₂, —(C₁-C₁₂)—CONHR, —(C₁-C₁₂)—CONR₂, —(C₁-C₁₂)—COSR, —(C₁-C₁₂)—COX, —(C₁-C₁₂)—S(O)₂NH₂, —(C₁-C₁₂)—S(O)₂NHR, —(C₁-C₁₂)—S(O)₂NR₂, —(C₁-C₁₂)—CN, —(C₁-C₁₂)—CR═NR, and —(C₁-C₁₂)—CONHNH₂ may be optionally substituted with halogen, amine, carboxylic acid, OR, CN, a C₁-C₁₂ thioester, and/or sulphonyl,
R is independently H or an optionally substituted C₁-C₁₂ alkyl group,
X is halogen, and [symbol]
is an optional double bond;
or an agriculturally acceptable salt thereof,
to the locus so as to thereby treat the locus against pest infestation,
wherein:
a) the compound of Formula I is applied free of fungal material from Pseudozyma aphidis,
b) the compound of Formula I is produced synthetically, or
c) the compound of Formula I is applied at an amount such that the locus being treated is exposed to an amount of the compound of Formula I that is higher than the amount the locus would be exposed to from secretions of Pseudozyma aphidis that exist naturally at the locus or is introduced artificially to the locus.

122. The method of claim 121, wherein:
R₁ is a C₁-C₁₂ alkyl optionally substituted with one or more substituents independently selected from the group consisting of halogen, CN, NH₂, —NH(C₁-C₄ alkyl), —(C₁-C₄ alkyl)—NH₂, —N(C₂-C₄ alkyl)(C₂-C₄ alkyl), OR, —S(O)₂ (C₁-C₄ alkyl), —S(O)(C₁-C₄ alkyl), C(O)OH and C(O)OR,
R₂ is —(C₁-C₁₂)—COOH, —(C₁-C₁₂)—CONH₂, —(C₁-C₁₂)—CONHR, —(C₁-C₁₂)—CONR₂, —(C₁-C₁₂)—COSR, —(C₁-C₁₂)—COX, —(C₁-C₁₂)—S(O)₂NH₂, —(C₁-C₁₂)—S(O)₂NHR or —(C₁-C₁₂)—S(O)₂NR₂, wherein each of the —(C₁-C₁₂)—COOH, —(C₁-C₁₂)—CONH₂, —(C₁-C₁₂)—CONHR, —(C₁-C₁₂)—CONR₂, —(C₁-C₁₂)—COSR, —(C₁-C₁₂)—COX, —(C₁-C₁₂)—S(O)₂NH₂, —(C₁-C₁₂)—S(O)₂NHR and —(C₁-C₁₂)—S(O)₂NR₂ may be optionally substituted with halogen, amine, carboxylic acid, OR, CN, a C₁-C₁₂ thioester, and/or sulphonyl,
R is independently H or an optionally substituted C₁-C₁₂ alkyl group,
X is halogen, and [symbol]
is an optional double bond.

123. The method of claim 121, wherein R₁ is a C₆-C₁₂ alkyl and/or R₂ is —(C₁-C₁₂)—COOH, —(C₁-C₁₂)—COO, or —(C₁-C₁₂)—COOR.

124. The method of claim 121, wherein R₁ is an octyl and/or R₂ is CH₂COOH.

125. The method of claim 121, wherein the compound of Formula I is selected from the group consisting of (E)-2-octyl-2-pentenedioic acid, (Z)-2-octyl-2-pentenedioic acid, and (E/Z)-2-octyl-2-pentenedioic acid.

126. The method of claim 121, wherein:
a) the compound of Formula I is extracted from a fungus,
b) the compound of Formula I is isolated or purified from the extract prior to application,
c) the compound of Formula I is applied free of fungal material, or
d) the compound of Formula I is produced synthetically.

127. The method of claim 121, wherein:
a) the pest infestation is a fungal infestation caused by at least one of Botrytis cinereal, Zymoseptoria tritici, Phytophthora infestans and Puccinia triticina,
b) the pest infestation is a bacterial infestation caused by at least one of Clavibacter michiganensis, Agrobacterium tumefaciens, Xanthomonas campestris p.v. phaseoli, Erwinia amylovora, Pseudomonas syringae pv. lachrymans, Pseudomonas syringae pv. tomato, Streptomyces scabies, Xanthomonas campestris pv. campestris and Xanthomonas capestris pv. Vesicatoria, or
c) the locus is a plant, plant propagation material, the vicinity of a plant, the vicinity of a propagation material of a plant, soil, seed of the plant, and/or foliage of the plant.

128. The method of claim 127, wherein the plant is a crop and wherein:
a) the compound of Formula I or agriculturally acceptable salt thereof is applied in the early stages of the crop cycle,
b) the compound of Formula I or agriculturally acceptable salt thereof is applied pre-sowing of the crop or post-sowing of the crop,
c) the compound of Formula I or agriculturally acceptable salt thereof is applied at a rate from 10 g/ha to 10000 g/ha, or
d) the compound of Formula I or agriculturally acceptable salt thereof is applied in a disease-inhibiting and agriculturally acceptable amount, preferably from about 0.1 to about 5000 ppm (parts per million).

129. The method of claim 121, wherein the method is effective for:
a) controlling fungal attack,
b) preventing fungal attack,
c) improving plant health,
d) improving the yield of a plant,
e) increasing biomass of the plant,
f) increased content of valuable ingredients in the plant,
g) improving vigor of the plant,
h) improving plant growth,
i) improving greenness of leaves,
j) improving quality of the plant, and/or
k) improving tolerance of the plant to abiotic and/or biotic stress.

130. A combination comprising:
a) (i) at least one compound of Formula I as defined in claim 121 or an agriculturally acceptable salt thereof, and (ii) at least one agriculturally acceptable carrier, wherein the concentration of the compound of Formula I in the combination is 0.03-1000 g/L,
b) (i) a composition comprising (1) at least one compound of Formula I as defined in claim 121 or an agriculturally acceptable salt thereof, and (2) at least one agriculturally acceptable carrier, wherein the concentration of the compound of Formula I in the composition is 0.03-1000 g/L, and (ii) at least one additional pesticide, or
c) (i) at least one compound of Formula I as defined in claim 121 or an agriculturally acceptable salt thereof, and (ii) at least one additional pesticide.

131. The combination of claim 130, wherein the combination is a composition comprising (i) at least one compound of Formula I as defined in claim 121 or an agriculturally acceptable salt thereof, and (ii) an agriculturally acceptable carrier, wherein the concentration of the compound of Formula I in the composition is 0.03-1000 g/L, and wherein:
a) the compound of Formula I is selected from the group consisting of (E)-2-octyl-2-pentenedioic acid, (Z)-2-octyl-2-pentenedioic acid, and (E/Z)-2-octyl-2-pentenedioic acid,
b) the agriculturally acceptable carrier is a solid carrier, a dusty agricultural carrier or a liquid carrier,
c) the composition comprises at least one surfactant and/or surface active dispersing agent,
d) the amount of the compound(s) of Formula I or agriculturally acceptable salt thereof in the composition is from about 0.1%, 0.5%, 1%, 1.5%, 2%, 2.5%, 3%, 3.5%, 4%, 4.5%, 5% to about 90%, 93%, 95%, 98%, 99% based on the total weight of the composition,
e) the composition has a pH of less than 7, preferably 3-6, and/or
f) the composition comprises at least one additional crop protection agents, preferably selected from the group consisting of insecticides, herbicides, fungicides, bactericides, nematicides, molluscicides, growth regulators, biological agents, fertilizers, and any mixture thereof.

132. A method for:
a) treating a locus against pest infestation comprising applying an effective amount of the combination of claim 130 to the locus so as to thereby treat the locus against pest infestation,
b) treating a plant or a plant material against pest infestation comprising applying an effective amount of the combination of claim 130 to the plant, the plant material, or the vicinity of the plant or plant material so as to thereby treat the plant or plant material against pest infestation,
c) controlling and/or preventing of fungal attack on soil, plant, root, foliage, seed, locus of the fungus, or a locus where fungal infestation is to be prevented, wherein the method comprises applying a fungicidally effective amount of the combination of claim 130 to the soil, plant, roots, foliage, seed, locus of the fungus, or locus in which the infestation is to be prevented so as to thereby control and/or prevent fungal attack,
d) treating a plant or a plant material against pest infestation comprising (i) obtaining the combination of claim 130, (ii) diluting the combination, and (iii) applying an effective amount of the diluted combination to the plant, the plant material, or the vicinity of the plant or plant material so as to thereby treat the plant or plant material against pest infestation, or
e) controlling and/or preventing of fungal attack on soil, plant, root, foliage, seed, locus of the fungus, or a locus where fungal infestation is to be prevented, wherein the method comprises (i) obtaining the combination of claim 130, (ii) diluting the combination, and (iii) applying an effective amount of the diluted combination to the soil, plant, roots, foliage, seed, locus of the fungus, or locus in which the infestation is to be prevented so as to thereby control and/or prevent fungal attack.

133. A process for:
a) preparing a crude extract comprising a compound of Formula I as defined in claim 121 or agriculturally acceptable salt thereof comprising the steps of:
(i) producing a conditioned culture medium of a fungus,
(ii) providing nitrogen-limiting conditions,
(iii) filtering the medium through a resin, and
(iv) eluting the adsorbed material from the resin with a polar solvent, or
b) extracting the compound of Formula I as defined in claim 121 from a fungus comprising:
(i) producing a conditioned culture medium of the fungus,
(ii) providing nitrogen-limiting conditions,
(iii) filtering the medium through a resin,
(iv) eluting the adsorbed material from the resin with a polar solvent, and
(v) isolating and purifying the compound of Formula (I) from the eluted material.

134. The process of claim 133, wherein:
a) the fungus is Pseudozyma aphidis,
b) the compound of Formula I is selected from the group consisting of (E)-2-octyl-2-pentenedioic acid, (Z)-2-octyl-2-pentenedioic acid, and (E/Z)-2-octyl-2-pentenedioic acid,
c) the polar solvent is selected from the group consisting of an alcohol, dimethylformamide, dimethylsulfoxide, acetonitrile, propylene carbonate, acetone and any combination thereof, preferably the polar solvent is methanol or ethanol, and/or
d) the resin is selected from the group consisting of amberlite resin, silica gel, alumina resin and any combination thereof, preferably the resin is amberlite resin.

135. A process for preparing a compound of formula (Ia) comprising: 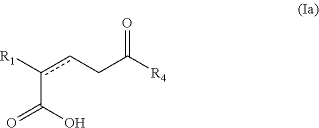
a) reacting a compound of formula (II) with compound of formula (III) in the presence of base and optionally in the presence of an organic solvent, 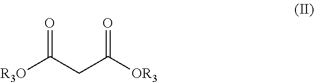
R₁X (III)
to form a compound of formula (IV), 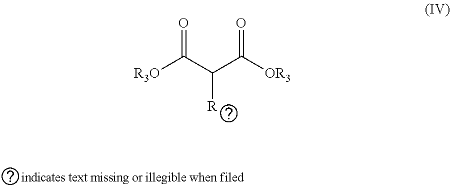
b) reacting the compound of formula (IV) with a compound of formula (V) in the presence of base and optionally in the presence of organic solvent 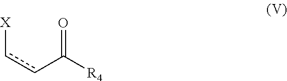
to form a compound of formula VI), and 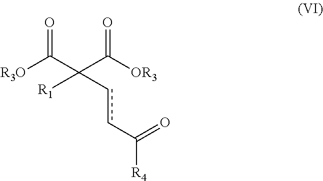
c) decarboxylating the compound of formula (VI) in the presence of catalyst and optionally in the presence of organic solvent to form the compound of formula (Ia),
wherein
R₁ is a C₁-C₁₂ alkyl optionally substituted with one or more substituents independently selected from the group consisting of halogen, C(O)R, CN, NH₂, —NH(C₁-C₄ alkyl), —(C₁-C₄ alkyl)—NH₂, —N(C₂-C₄ alkyl)(C₂-C₄ alkyl), OR, —S(O)₂ (C₁-C₄ alkyl), —S(O)(C₁-C₄ alkyl), C(O)OH and C(O)OR;
R₃ is hydrogen or C₁-C₄ alkyl;
R₄ is OH, —O—, —NH₂, —NH—NH₂, —NH(C₁-C₁₂ alkyl), —N(C₂-C₄ alkyl)(C₂-C₄ alkyl), —O(C₁-C₁₂ alkyl), halogen, SR₃, or S(O) R₃;
X is bromine, chlorine or fluorine; and
R is independently H or an optionally substituted C₁-C₁₂ alkyl group.

136. The process of claim 135, wherein:
R₁ is a C₁-C₁₂ alkyl optionally substituted with one or more substituents independently selected from the group consisting of halogen, CN, NH₂, —NH(C₁-C₄ alkyl), —(C₁-C₄ alkyl)—NH₂, —N(C₂-C₄ alkyl)(C₂-C₄ alkyl), OR, —S(O)₂ (C₁-C₄ alkyl), —S(O)(C₁-C₄ alkyl), C(O)OH and C(O)OR;
R₃ is hydrogen or C₁-C₄ alkyl;
R₄ is OH, —O(C₁-C₁₂ alkyl), halogen, —NH(C₁-C₁₂ alkyl), SR₃, or S (O) R₃;
X is bromine, chlorine or fluorine; and
R is independently H or an optionally substituted C₁-C₁₂ alkyl group.

137. The process of claim 135, wherein:
a) the base is selected from the group consisting of alkali metal salt of hydrides, alkali metal salt of carbonates, alkali metal salt of alkoxides, alkali metal salt of C₁-C₄ alkyls and the mixtures thereof,
b) the process is performed in the absence of organic solvent or the process is performed in the presence of at least one organic solvent wherein the organic solvent is selected from the group consisting of C₁-C₄ alcohols, ethers, C₁-C₄ alkyl esters, lactones, amides, sulfoxides, aliphatic and aromatic carbohydrates, and the mixtures thereof, and/or
c) (i) the compound of formula (Ia) is (Z)-isomer and is substantially free of (E)-isomer of compound of formula (Ia), (ii) the compound of formula (Ia) is (E)-isomer and is substantially free of (Z)-isomer of compound of formula (Ia), or (iii) the compound of formula (Ia) is (E)-isomer, (Z)-isomer or a mixture thereof, and/or
d) the catalyst is an inorganic acid selected from the group consisting of HCl, H₂SO₄, and a mixture thereof or the catalyst is an organic acid selected from the group consisting of carboxylic acids, amines and mixtures thereof.

138. A process for preparing 2-octyl-2-pentenedioic acid or a salt thereof comprising:
a) reacting diethyl malonate with 1-halooctane to obtain diethyl 2-octylmalonate or a salt thereof;
b) reacting the obtained diethyl 2-octylmalonate with ethyl-3-haloacrylate to form triethyl-undec-1-ene-1,3,3-tricarboxylate or a salt thereof; and
c) converting the formed triethyl-undec-1-ene-1,3,3-tricarboxylate to obtain 2-octyl-2-pentenedioic acid.

139. The process of claim 138, wherein:
a) (i) the obtained 2-octyl-2-pentenedioic acid is (E)-2-octyl-2-pentenedioic acid, (Z)-2-octyl-2-pentenedioic acid, (E/Z)-2-octyl-2-pentenedioic acid, or any mixture thereof, (ii) the obtained 2-octyl-2-pentenedioic acid is (Z)-2-octyl-2-pentenedioic acid, (iii) the obtained 2-octyl-2-pentenedioic acid is (Z)-2-octyl-2-pentenedioic acid and is substantially free of (E)-2-octyl-2-pentenedioic acid, or (iv) the obtained 2-octyl-2-pentenedioic acid is (E)-2-octyl-2-pentenedioic acid and is substantially free of (Z)-2-octyl-2-pentenedioic acid,
b) the 1-halooctane is selected from the group consisting of 1-bromooctane, 1-chlorooctane, and 1-fluorooctane,
c) the ethyl-3-haloacrylate is selected from the group consisting of ethyl-3-bromoacrylate, ethyl-3-chloroacrylate, and ethyl-3-fluoroacrylate,
d) step a) is carried out in the presence of a base, preferably selected from the group consisting of alkali metal salt of hydrides, alkali metal salt of carbonates, alkali metal salt of alkoxides, alkali metal salt of C₁-C₄ alkyls, and mixtures thereof,
e) e conversion is carried out in the presence of an inorganic acid selected from the group consisting of HCl, H₂SO₄ and mixtures thereof, or the conversion is carried out in the presence of an organic acid selected from the group consisting of carboxylic acids, amines, and mixtures thereof, and/or
f) wherein the conversion step is carried out at pH of about 1-4.

140. A method of:
a) treating a locus against pest infestation comprising (i) preparing a crude extract comprising a compound of Formula I or agriculturally acceptable salt thereof using the process of claim 133 or (ii) extracting the compound of Formula I using the process of claim 133, and applying an effective amount of the crude extract or the compound of Formula I to the locus so as to thereby treat the locus against pest infestation,
b) treating a plant or a plant material against pest infestation comprising (i) preparing a crude extract comprising a compound of Formula I or agriculturally acceptable salt thereof using the process of claim 133 or (ii) extracting the compound of Formula I using the process of claim 133, and applying an effective amount of the crude extract or the compound of Formula to the plant, the plant material, or the vicinity of the plant or plant material so as to thereby treat the plant or plant material against pest infestation, or
c) controlling and/or preventing of fungal attack on soil, plant, root, foliage, seed, locus of the fungus, or a locus where fungal infestation is to be prevented comprising (i) preparing a crude extract comprising a compound of Formula I or agriculturally acceptable salt thereof using the process of claim 133 or (ii) extracting the compound of Formula I using the process of claim 133 and applying an effective amount of the crude extract or the compound of Formula I to the soil, plant, roots, foliage, seed, locus of the fungus, or locus in which the infestation is to be prevented so as to thereby control and/or prevent fungal attack.
